(19) United States
(12) Patent Application Publication
(10) Pub. No.: US 2026/0071176 A1
(43) Pub. Date: Mar. 12, 2026

(54) Universal Efficient Dextran Microparticles Production with Microfluidic Technology

(71) Applicants: Sanofi, Paris (FR); Institut Pasteur, Paris (FR); Commissariat a L'Energie Atomique et Aux Energies Alternatives, Paris (FR)

(72) Inventors: Guillaume Mottet, Paris (FR); Viacheslav Rusanov, Paris (FR); Samy Gobaa, Paris Cedex (FR); Isabelle Texier-Nogues, Grenoble Cedex 09 (FR); Antoine Hoang, Grenoble Cedex 09 (FR)

(21) Appl. No.: 19/287,161

(22) Filed: Jul. 31, 2025

(30) Foreign Application Priority Data
Aug. 1, 2024 (EP) 24306301.3

(51) Int. Cl. C12N 5/00; A61K 9/50; A61K 35/12; B01L 3/00; G01N 33/50

(52) U.S. Cl. CPC C12N 5/0075; A61K 9/5031; A61K 9/5036; A61K 35/12; B01L 3/502761; C12N 5/0012; G01N 33/5026; G01N 33/5082; C12N 2503/02; C12N 2513/00; C12N 2531/00; C12N 2533/30; C12N 2533/70; C12N 2537/10; G01N 2500/10

(57) ABSTRACT

The present invention relates to the use of modified dextran hydrogel microparticles comprising (i) at least one vinyl sulfone functionalized dextran, and (ii) at least one crosslinkable polymer having at least two thiol functions, wherein the dextran has a molecular weight comprised between 5 and 500 kDa, and the substitution degree of the dextran by the vinyl sulfone is comprised between 5 and 60%, for encapsulating at least one synthetic or natural cell. The present invention also relates to modified dextran hydrogel microparticles, a process for preparing them, an in vitro method for cultivating at least one cell comprised in said microparticles, in vitro methods for screening, for producing or for testing compounds, a kit, a microfluidic or millifluidic channel, a process for encapsulating said microparticles, and a method for the quality control of a batch. Said microparticles are useful in the field of biological and medical applications.

### CLAIM PRIORITY

This application claims priority to Application No. EP24306301.3, filed on Aug. 1, 2024, the disclosure of which is incorporated herein by reference.

### FIELD OF THE DISCLOSURE

Disclosed herein are methods for preparing cell encapsulated modified dextran hydrogel microparticles, by microfluidic technology, said microparticles and their uses in the field of biological and medical applications, such as the production of spheroids, for example for high throughput drug screening or three-dimensional cell culture.

### BACKGROUND OF THE DISCLOSURE

Hydrogel microbeads have found applications in various areas, including biological and pharmaceutical applications such as drug delivery as well as screening technology.

Said hydrogel microbeads may typically be used for three-dimensional (3D) cell culture, which is a powerful tool for studying cellular behavior, disease mechanisms, and therapeutic interventions in a more realistic context than two-dimensional (2D) cell culture, i.e. providing a more physiologically relevant environment that closely mimics in vivo tissue conditions. In this context, spheroids may be formed wherein cell may grow in all directions, creating a 3D architecture that promotes more natural cell-cell and cell-extracellular matrix (ECM) interactions.

Numerous polymer compounds have been used to form the backbone of hydrogel microbeads such as alginate, agarose, dextran, PEG, hyaluronic acid, PVA, etc. Dextran has the advantages of being biocompatible, transparent, soft, and elastic. Moreover, in pure form dextran does not interfere with cells and can be biodegraded by dextranase. One other major compound to make the hydrogel structure is the linker. The linker enables the jellification of the structure and has a direct influence on the physical and biological property of the hydrogel, such as hardness, porosity, elasticity and cell adhesivity such as for example RDG-SH. Numerous linker compounds have been used, such as PEG, DTT, dextran, gelatin, collagen, proteins, block-polymers etc. To bond the polymer and the linker together, different chemistries have already been proposed, such as Thiol/VS (vinyl sulfone), Maleimide/Thiol, Methacry late/Methacrylate, etc.

Chung et al. (ACS Appl. Bio. Mater., 2021, droplet-based microfluidic synthesis of hydrogel microparticles via click chemistry-based cross-linking for the controlled release of proteins) describes a droplet-based microfluidic synthesis method for the fabrication of monodisperse hydrogel microparticles (HMPs) via click cross-linking between VS (vinyl sulfone) and SH (thiol) polymers, in particular HMPs comprising a dextran vinyl sulfone having a molecular weight of 40 kDa (dextran40k-VS) and a substitution degree (also named DS, degree of modification or DM) of the dextran by the vinyl sulfone of 6% or 12%, and a dextran-SH having a molecular weight of 40 kDa. However, these HMPs are used for drug delivery, in particular for Bevacizumab delivery.

Hiemstra et al. (Macromolecules, 40, 1165-1173, 2007, Novel in situ forming, degradable dextran hydrogels by Michael addition chemistry: Synthesis, Rheology, and Degradation) describes dextrans functionalized with vinyl sulfone groups linked by a hydrolytically susceptible ester bond which are synthesized by a one-pot synthesis procedure to a broad range of degrees of substitution and hydrogels which are rapidly formed in situ under physiological conditions by mixing aqueous solutions of vinyl-sulfone functionalized dextrans and multifunctional mercapto-PEG. However, these hydrogels are self-degradable since they degrade within 3-21 days (degradation of the ester bond) and thus these dextran hydrogels are not compatible with cell culture. In addition, no biological application is shown.

Bavli et al. (Developmental Cell 56, 1804-1817, 2021, CloneSeq: a highly sensitive analysis platform for the characterization of 3D-cultured single cell-derives clones) discloses hydrogel beads comprising maleimide dextran (named MALDEX) and polyethylene glycol dithiol (named PEGDT or SH-PEG-SH) as a crosslinker for encapsulating single cells by using microfluidic devices. However, microfluidic production of these beads, lack of stability, exercising problem of gel polymerization in the microfluidics channel, as well as aggregation of beads after emulsion breaking. In addition, it does not allow prolongated cell culture because of the degradability of thiol-maleimide bonds, which is inherent of the chemistry used.

US2022/220228 describes a crosslinked dextran polymer, bearing carboxy late groups, a hydrogel comprising this crosslinked dextran polymer and optionally biological cells, as well as an implant comprising this hydrogel for the treatment of chronic diseases by replacing totally or in part the function of natural occurring cells which are deficient in a patient. Thus, this dextran polymer is not a pure dextran functionalized by vinyl sulfone as it is crosslinked and bears carboxylate groups. In other terms the dextran polymer described in US2022/220228 is not a hydroxyl-containing water-soluble polymer which does not bear carboxylate groups and more particularly it is not a dextran which has been functionalized by vinyl sulfone groups via a one-step reaction method. In addition, US2022/220228 discloses only bulk hydrogels-millimeter-scale slabs and discs made by pouring mixtures into molds and not hydrogel microparticles or microbeads having a size ranging from 1 to 1000 μm. Furthermore, US2022/220228 is totally silent about droplet microfluidic methods.

Thus, despite the ongoing research and development in the area of hydrogel microbeads there is still a need for the development of hydrogel microbeads and methods for manufacturing them allowing the obtention of hydrogel microbeads in high yield, that could be biocompatible, accessible for standard laboratory manipulation technics, as pipetting, centrifugation, distribution in well plates, as well as more complex analysis methods like FACS (Fluorescence-activated cell sorting), immunofluorescence (IF) analysis (immunostaining), as well as secretome and transcriptomics analysis (RNA extraction, RNA sequencing). In particular, there is still a need of developing microbeads that allow prolongated cell proliferation, under the mild conditions, without important structure changes, as well as microbeads that allow protein and growth factor diffusion and that could be produced keeping low coefficient of size variation.

Furthermore, there is still a need of developing microbeads which have a very good transparency in particular for imaging technics, and which can be dissolved in a biocompatible manner so as to access microbead load.

Further, there is still a need of providing microbeads which could have modulable stiffness and that allow to produce a wide variety of spheroids which exhibit a high level of manipulability, in particular which are obtained in a greater quantity than would be obtained for example by using Ultra-low attachment (ULA) plate, and which are thus useful for universal applications.

In addition, there is still a need of developing hydrogel microbeads having adapted mechanical properties, in particular for enabling to adapt the hydrogel stiffness depending on the cell origin and improve the survival and/or the growing of the cells of interest.

Furthermore, there is still a need of providing hydrogel microbeads which allow to produce both physiologically and non-physiologically structured organoids while facilitating large-scale organoid production.

Further, there is still a need of providing hydrogel microbeads which support cell viability, adherence, and the formation of embryoid bodies, while maintaining pluripotency.

Further, there is still a need of providing hydrogel microbeads which allow spheroid reinjections.

Besides, when encapsulating cells in hydrogel beads during polymerization, gravity causes the cells to settle near the bead walls. This leads to two problems. First, beads containing cells become less stable, and even during standard pipetting, cells may escape from the beads. Second, when a spheroid grows from cells located near the bead's edge, the growing spheroid quickly destroys the bead structure and escapes from it.

Although there are well-known already proposed methods, there is still a need of providing methods to obtain hydrogel microbeads which enable cell centering and clustering of transmembrane proteins.

### SUMMARY OF THE DISCLOSURE

Provided herein is the use of modified dextran hydrogel microparticles comprising (i) at least one vinyl sulfone functionalized dextran, and (ii) at least one crosslinkable polymer having at least two thiol functions, wherein
the dextran has a molecular weight comprised between 5 and 500 kDa, and
the substitution degree of the dextran by the vinyl sulfone is comprised between 5 and 60%,
   for encapsulating at least one synthetic or natural cell.

### Herein Provided are Also:

Modified dextran hydrogel microparticles comprising (i) at least one vinyl sulfone functionalized dextran, (ii) at least one crosslinkable polymer having at least two thiol functions and (iii) at least one synthetic or natural cell, wherein
   the dextran has a molecular weight comprised between 5 and 500 kDa, and
   the substitution degree of the dextran by the vinyl sulfone is comprised between 5 and 60%,
provided that the at least one crosslinkable polymer having at least two thiol functions is different from a crosslinkable polymer comprising dextran having a molecular weight comprised between 30 and 50 kDa as the polymer chain:
   Modified dextran hydrogel microparticles comprising (i) at least one vinyl sulfone functionalized dextran, (ii) at least one crosslinkable polymer having at least two thiol functions and (iii) at least one synthetic or natural cell,
wherein
   the dextran has a molecular weight comprised between 5 and 500 kDa,
   the substitution degree of the dextran by the vinyl sulfone is comprised between 5 and 60%, and
   the crosslinkable polymer comprises
      at least a polymer chain selected from a group consisting of PEG, gelatin, agarose, alginate, hyaluronic acid, collagen, proteins, polyacrylamide, block-polymers,
      at least an (C4-C12)alkylene chain, optionally substituted by one or two hydroxy group(s), or
      their mixtures,
   in particular PEG:
A process for preparing said modified dextran hydrogel microparticles as defined in the present disclosure, wherein it comprises at least a step of using a microfluidic device, in particular comprising at least two inlet channels that converge into a droplet generation region:
Modified dextran hydrogel microparticles comprising (i) at least one vinyl sulfone functionalized dextran, (ii) at least one crosslinkable polymer having at least two thiol functions and (iii) at least one synthetic or natural cell obtainable by a process as defined in the present disclosure:
An in vitro method for cultivating (or culturing) at least one cell comprised in the modified dextran hydrogel microparticle as defined in the present disclosure, comprising at least a step of incubating the microparticle in an environment, suitable to allow for cell survival, cell growth, cell differentiation and/or cell proliferation such as clonal expansion, more particularly for producing a cell cluster, a spheroid, an organoid, a gastruloid, a tumoroid or a tissue:
An in vitro method for screening compounds, proteins, polypeptides, oligopeptides, cell secretome or antibodies comprising using a modified dextran hydrogel microparticle comprising cultivated cells according to the in vitro method as defined in the present disclosure:
A kit for making the microparticle encapsulating at least one synthetic or natural cell as defined in the present disclosure, the kit comprising: at least one vinyl sulfone functionalized dextran, a culture medium: at least one crosslinkable polymer having at least two thiol functions: an oily phase: a microfluidic chip; and optionally instructions for use:
An in vitro method for producing compounds, proteins, polypeptides, oligopeptides, nucleic acids, cell secretome or antibodies from at least one synthetic or natural cell encapsulated in the modified dextran hydrogel microparticle(s) as defined in the present disclosure, comprising at least:
   a step of cultivating and/or of operational maintaining of the at least one synthetic or natural cell: or
   a step of production of said compounds, proteins, polypeptides, oligopeptides, nucleic acids, cell secretome or antibodies from said at least one synthetic or natural cell: or
   a step of cultivating and/or of operational maintaining of the at least one synthetic or natural cell, and a step of production of said compounds, proteins, polypeptides, oligopeptides, nucleic acids, cell secretome or antibodies from said at least one synthetic or natural cell:
A microfluidic or millifluidic circuit or channel comprising the modified dextran hydrogel microparticles as defined in the present disclosure:
A process for encapsulating in a compartment, in particular in an aqueous or in a hydrogel droplet, a modified dextran hydrogel microparticles as defined in the present disclosure comprising at least one step of injection of said modified dextran hydrogel microparticles in a microfluidic or millifluidic circuit or channel:
A modified dextran hydrogel microparticle in accordance with the present disclosure encapsulated in a compartment, in particular in an aqueous or a hydrogel droplet:
A method for the quality control of a batch of modified dextran hydrogel microparticles as defined in the present disclosure, comprising at least:
   a step of recovering of a sample of microparticles from said batch:
   a step of measuring at least one parameter for each microparticle of said sample:
   a step of comparison of the value of the at least one measured parameter for each microparticle of said sample with a predetermined value:
   a step of determination based on said comparison whether or not each microparticle of said sample has the required quality; and
   a step of extrapolation of the results of the previous determination step to said batch:
A pharmaceutical composition for use in a method of treating a patient having a disorder or condition, and/or for transplantation therapy, and/or for use to restore and/or improve the function of a tissue or of an organ of a patient, and/or for use for tissue regeneration and/or tissue repair,
said pharmaceutical composition comprising the cells cultivated according to the in vitro method for cultivating at least one cell comprised in the modified dextran hydrogel microparticle as defined in the present disclosure:
An in vitro method for testing a drug compound comprising using at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof, produced in a modified dextran hydrogel microparticles as defined in the present disclosure; and
An in vitro method for testing a drug compound comprising at least:
   a step of culturing at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof, produced according to the in vitro method of the present disclosure,
   a step of exposing said at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof, to the drug compound,
   a step of measuring a biological response of interest, and
a step of comparison of the biological response with a predetermined value or with a control condition wherein said at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof, has not been exposed to said drug compound, thereby assessing the biological response of the tested drug compound.

In the present disclosure, the term “culturing” (or “cultivating”), in addition to the cultivation of a single cell type in a controlled environment (monoculture), comprises “co-culturing” (or “co-cultivating”) that is to say the culturing of from 2 to 1 000 species, for example from 2 to 500 species, and in particular 2 species, such as a yeast and a Jurkat cell or a microbiota and a gut organoid.

### Definitions

In the context of the present disclosure, the terms below have the following definitions unless otherwise mentioned throughout the instant specification:
“hydrogel” means a gel-like material composed of a polymeric network and a substantial quantity of water. It is a three-dimensional network of hydrophilic (water-loving) polymer chains that are crosslinked to form a stable gel structure. This material is biocompatible, meaning it is compatible with living cells and tissues and can be used in medical and biological applications without causing adverse reactions. Hydrogels possess tunable properties such as stiffness, permeability, and responsiveness to external stimuli (e.g., pH, temperature) and can encapsulate cells, micro and nanoparticles, molecules within their structure.
“cell” refers to a natural cell, a genetically modified derivative of a natural cell, biopsy cell or a synthetic cell. By way of examples, but not limited to, among the cells in accordance with the present disclosure may be mentioned eukaryotic cells such as a mammalian cells, for instance human cells, prokaryotic cells such as bacterial cells or archaea cells, cancerous cells, tumoral cells, somatic cells, spleen cells, stem cells, progenitor cells, precursor cells, fully differentiated cells, undifferentiated cells, germ cells, cells of unicellular organism or of multi-cellular organism, fungal cells, splenocytes and hybridoma cell, connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof.
“natural cell” refers to a primary cell, immortalized cell line, or stem cell derived from a living organism. a biopsy sample cell taken from a living organism, a PDX cell, a CDX cell, a PDO cell, or a dissociated organoid cell.
“synthetic cell” means a cell which functions as biological mimics of natural cells by mimicking salient features of cells such as metabolism, response to stimuli, gene expression, direct metabolism, and high stability.
“cell-free system” designates an experimental set up that involves the use of cellular components outside of living cells. Cell-free systems typically include extracts from cells that contain necessary cellular machinery such as enzymes, ribosomes, amino acids etc. required for specific biochemical reactions. These systems are used to study and manipulate biological processes in a simplified and controlled context.
“Dextran” means a polyhydroxy compound which is a complex branched molecule called a glucan, which means it is a polysaccharide (sugar molecule chain) made up of linked glucose units. Dextran is a natural water-soluble polymer which has a variable formula due to its chain length variation, but it can be generally represented as H(C₆H₁₀O₅)ₓOH, where x represents the number of glucose units.
“Substitution degree (DS) or degree of modification (DM) of the dextran refers to the average number of monomers in the dextran backbone that have been modified by substituting their hydroxyl groups with a different functional group.
“PEG” means polyethylene glycol. Also known by other names like polyethylene oxide (PEO) or polyoxyethylene (POE), depending on its molecular weight. Its chemical formula is C2nH4n+2On+1 (where n represents the number of repeating units) and it is soluble in water and many other solvents.
“Linker” or “crosslinker” means molecule that connects two other molecules or chemical groups, forming stable bonds between them. These bonds can be covalent or non-covalent such as hydrogen bonds, depending on the nature of the interaction required for the specific application.
“microbeads” is defined by beads with a radius of 1 to 999 μm.
“crosslinkable polymer” refers to a polymer modifiable with a linker (or a crosslinker) during a crosslinking reaction.
“microfluidic” Refers to use of channels and chambers designed to handle and manipulate liquids at low Reynolds numbers (<2000-laminar flow).
“millifluidic” Refers to use of channels and chambers having at least one millimetric dimension of its cross-section.
“droplet” refers to water-in-oil droplet having any shape, for example cylindrical, spherical, ellipsoidal, irregular shapes, etc. Generally, in emulsions of the disclosure, aqueous droplets are spherical or substantially spherical in a fluorocarbon, continuous phase.
“droplet microfluidic” refers to the technology of manipulation and production of droplets of liquid with microfluidic device, that composed of channels and chambers to handle and manipulate fluids at low Reynolds numbers (<2000)
a “spheroid” refers to three-dimensional (3D) cellular aggregate. Spheroids may be created within the hydrogel microbeads, which serve as a scaffold or support matrix which offers a controlled environment for their growth and function.
a “cell cluster” refers to a group of cells that are adherent to each other, existing as a distinct aggregate within the microfluidic channels or chambers.
an “organoid” refers to a miniaturized, three-dimensional (3D) structure that resembles a specific human organ or tissue. These organoids may be cultured within the hydrogel microbeads which offer a controlled environment for their growth and function.
a “gastruloid” refers to a three-dimensional cellular structure than can be cultured within the hydrogel microbeads that mimics certain aspects of early embryonic development, particularly the process of gastrulation.
a “tumoroid” refers to three-dimensional (3D) tumor-like structures or spheroids that are cultured within the hydrogel microbeads. A tumoroid is thus a 3D aggregate of cancer cells that form spherical or irregular shapes within the hydrogel beads.

The function and uses of such spheroid, organoid, gastruloid and tumoroid are detailed herein after in the PARAGRAPH “IN VITRO METHODS”.
a “tissue” means a naturally occurring collection of different cell types organized together to perform a specific function.
“secretome” refers to all the molecules secreted by a cell or drop off from its membrane.

The secreted molecules include proteins, protein fraction, lipids, miRNAs, metabolites, messenger-RNAs, DNA, exosomes or microvesicles.
“Young's modulus” and “shear modulus” are related by E=2G (1+v) (for isotropic and homogeneous materials), in which E is Young's modulus, G is shear modulus and v is Poisson's ratio.
“Jellification” refers to the transformation of a sol (a colloidal solution) into a gel, where the material forms a semi-solid network that traps the solvent within its structure.
“Polymerization” involves the chemical reaction where monomer or polymer molecules linked together to form polymer chains or block polymer chains or networks, contributing to the sol-gel transition by creating a more interconnected and stable structure.

In the framework of the present disclosure, the term “jellification” or “polymerization” may be used interchangeably.
“Functional assay” is any assay that can be performed by the skilled person on a cell or a biological agent to access its functionality such as its biological functionality. Among the functional assays can be cited for example, a reporter assay (for instance bioluminescent, fluorescent, etc.), for example the protein fluorescence production following the activation of a cell pathway such as NFκB.
“Drug compound” can be any compound selected from small molecules, proteins, polypeptides, peptides, amino acids, gene edited constructs, polymeric carriers, dendrimers, extracellular vesicles, microbial therapeutics, contrast agents, diagnostic probes, synthetic polymers, synthetic copolymers, nucleic acids, DNA, RNA, antibodies, antibody fragments, oligopeptides, viruses, viral and virus like particles, LNP (lipid nanoparticle), metal particles, inorganic particles, nanoparticles, photopharmaceuticals, photosensitizers, and combinations thereof.

In the framework of the present disclosure, the term “hydrogel microparticles” or “hydrogel microbeads” or “hydrogel beads” may be used interchangeably. As mentioned above, the “hydrogel microparticles” in accordance with the present disclosure can be used as a support matrix for the encapsulated cells which are included therein.

### BRIEF DESCRIPTION OF THE FIGURES

FIG. 1A represents tumoroids (or spheroids) produced according to the present disclosure and obtained from MKN45 cells, 7 days after encapsulation in the modified dextran hydrogel microparticles according to the present disclosure and by using a 2.8% (w/v) gel obtained by using a Dextran-VS (DS=22%) (see example paragraph 1.2 below). The scale bar is 200 μm.

FIG. 1B represents tumoroids (or spheroids) produced according to the present disclosure and obtained from HEP3B cells, 14 days after encapsulation in the modified dextran hydrogel microparticles according to the present disclosure and by using a 2.8% (w/v) gel obtained by using a Dextran-VS (DS=22%) (see example paragraph 1.3 below). The scale bar is 200 μm.

FIG. 1C represents tumoroids (or spheroids) produced according to the present disclosure and obtained from IGROV1 cells, 14 days after encapsulation in the modified dextran hydrogel microparticles according to the present disclosure and by using a 3.5% (w/v) gel obtained by using a Dextran-VS (DS=22%) (see example paragraph 1.4 below). The scale bar is 200 μm.

FIG. 1D represents tumoroids (or spheroids) produced according to the present disclosure and obtained from MDA-MB-468 cells, 5 days after encapsulation in the modified dextran hydrogel microparticles according to the present disclosure and by using a 3.3% (w/v) gel obtained by using a Dextran-VS (DS=22%) (see example paragraph 1.5 below). The scale bar is 200 μm.

FIG. 2A represents Dextran-VS (DS=22%)-SH-PEG-SH gel rheometric measurements, during polymerization at 15° C. for 2.8% (w/v) (dotted line) and 3.5% (w/v) (dot dotted line) gels (graph on the left), and an end point result for the polymerization of 5% (w/v) gel, polymerized at 25° C. (graph on the right). More particularly, in the graph on the left, the x-axis represents Time in second(s) and the y-axis represents Shear modulus in Pascal (Pa). The curve at the bottom illustrates a 2.8% (w/v) gel and the curve at the top illustrates a 3.5% (w/v) gel. And the shading around the lines represents standard deviation. In the graph on the right, the x-axis represents 5% (w/v) gel Dextran-VS and the y-axis represents Shear modulus in Pascal (Pa) (see example paragraph 1.6 below).

FIG. 2B represents tumoroids (or spheroids) produced according to the present disclosure and obtained from MKN45 cells, 10 days after encapsulation in the modified dextran hydrogel microparticles according to the present disclosure and by using a 10% (w/v) gel obtained by using a Dextran-VS (DS=22%) (see example paragraph 1.6 below). The scale bar is 200 μm.

FIG. 2C represents tumoroids (or spheroids) produced according to the present disclosure and obtained from MKN45 cells, 7 days after encapsulation in the modified dextran hydrogel microparticles according to the present disclosure and by using a 3.3% (w/v) gel obtained by using a Dextran-VS (DS=22%) (see example paragraph 1.6 below). The scale bar is 200 μm.

FIG. 2D represents tumoroids (or spheroids) produced according to the present disclosure and obtained from MKN45 cells, 7 days after encapsulation in the modified dextran hydrogel microparticles according to the present disclosure and by using a 2.8% (w/v) gel obtained by using a Dextran-VS (DS=22%) (see example paragraph 1.6 below). The scale bar is 200 μm.

FIGS. 2E, 2F and 2G represent tumoroids (or spheroids) produced according to the present disclosure and obtained from IGROV1 cells in Dextran gel at different w/v ratios, respectively from the left to the right at 5%, 3.5% and 2.8% (w/v) gel obtained by using a Dextran-VS (DS=22%) with the addition of 100 μM of SH-RGD. (see example paragraph 1.6 below). The scale bar is 200 μm.

FIG. 3A represents a graph which comprises curve of EC50 test. The duration of the test was 96 hours, R²=0.98 and EC50=1 nM. The curb was obtained on MKN45 spheroids in gel shells with CellTiter-GloR 2.0 Cell Viability Assay. The x-axis corresponds to ADC (antibody-drug conjugate) log 10 (concentration), M and the y-axis corresponds to RLU (relative luminescence units) (see example paragraph 1.7 below).

FIG. 3B represents the frequency distribution of droplet's sizes based on analysis of frequency of droplet production. More particularly, the size analysis of 200 000 monodisperse droplets during their production which comprise cells encapsulated and prepared according to the present disclosure was carried out. The x-axis corresponds to droplet size in μm (micrometer) and the y-axis corresponds to number of droplets. The Coefficient of Variation is equal to 1.3% and the mean is 80 μm (see example paragraph 1.8 below).

FIG. 3C represents a Volcano Plot of Gene expression changes 3D (spheroids) versus 2D. More particularly, this figure illustrates an example of NGS (Next-Generation Sequencing) RNAseq data obtained from comparison of MKN45 spheroids and standard 2D cultured MKN45 cells. In the graph, the x-axis represents Fold Change (−log₂) and the y-axis represents P-value (−log₁₀). The horizontal line represents a P-value equal to 0.05. Total amount of genes with fold changes more than 2 and P-value less than 0.05 is 1529 (see example paragraph 1.9 below).

FIG. 3D represents a Volcano Plot of Gene expression changes 3D (spheroids) versus 2D. More particularly, this figure illustrates an example of NGS RNAseq data obtained from comparison of IGROV1 at 14-day-old spheroids and standard 2D cultured IGROV1 cells. In the graph, the x-axis represents Fold Change (−log₂) and the y-axis represents P-value (-log₁₀) The horizontal line represents a P-value equal to 0.05. Total amount of genes with fold changes more than 2 and P-value less than 0.05 is 2175 (see example paragraph 1.9 below).

FIG. 3E represents a graph which comprises curve of EC50 test. The duration of the test was 72 hours, R²=0.93 and EC50=0.54 nM. The curb was obtained on MKN45 spheroids in gel shells with CellTiter-Glo® 2.0 Cell Viability Assay. The x-axis corresponds to ADC (antibody-drug conjugate) log₁₀ (concentration), nM and the y-axis corresponds to Relative Cell Viability (see example paragraph 1.7 below).

FIG. 4 represents four confocal images of 14 days-old IGROV1 spheroids obtained according to example paragraph 1.4 and stained using the protocol described in example paragraph 1.10. The DNA-staining (DAPI, that is to say 4,6-diamidino-2-phenylindole,), F-actin (named filamentous actin or actin filaments: Phalloidin was used to stain actin-F), E-Cadherin, and Ki67 is shown respectively from the left to the right. Scale bar is 50 μm.

FIG. 5A represents two microscope images of the same hydrogel microbeads obtained by using a Dextran-VS (DS=22%): one in bright field and one in fluorescent (red). On the top image, hydrogel microbeads are observed, some of them comprise CHO cells inside and others are empty. On the top image, the multiple dark dots inside the hydrogel beads are magnetic beads. On the bottom image, for some hydrogel beads, the IgG antibodies detection can be identified through the fluorescent signal on the magnetic beads (see example paragraph 2.5 below).

FIG. 5B represents a Flow cytometer plot of Forward scattering (FSC-H: x-axis) and Side scattering (SSC-H: y-axis) of Hydrogel Beads. Two groups are well distinct: the hydrogel beads with CHO cells (grey dots) and the ones empty (dark dots) (see example paragraph 2.5 below).

FIG. 5C represents a Flow cytometer plot of red fluorescent signal (IgG secretion, anti-Fc Alexa 647) and green fluorescent signal (CHO detection via Calcein AM) of a pool of CHO cells encapsulated in hydrogel beads. This graph was divided into four rectangles. Each rectangle was named Qx in which x is 1 to 4. Q1 is the rectangle located on the left at the top, Q2 is the rectangle located on the right at the top, Q3 is the rectangle located on the right at the bottom, and Q4 is the rectangle located on the left at the bottom. On the Q4 rectangle, empty hydrogel beads were observed. On the Q1 rectangle, hydrogel beads with CHO cells that do not produce IgG or very few IgG were observed. On the Q2 rectangle, hydrogel beads with CHO cells that secreted IgG were observed. On the Q3 rectangle, hydrogel beads with CHO cells that were dead but had enough time to secret IgG or empty hydrogel beads that were contaminated with IgG coming from other beads containing IgG secreting CHO cells were observed. In the graph, the x-axis represents IgG secretion (anti-Fc) and the y-axis represents CHO detection (Calcein AM) (see example paragraph 2.5 below).

FIG. 5D represents a Flow cytometer plot of red fluorescent signal (IgG secretion) versus the number of events of two CHO samples: one from a high IgG producer clone (dark grey color) and one from CHO pool meaning various IgG producers (light grey color,). In the graph, the x-axis represents IgG secretion (anti-Fc) and the y-axis represents Count (see example paragraph 2.5 below).

FIG. 6 provides a schematic illustration of one embodiment of the process according to the present disclosure, wherein an extraction step (step (e) as detailed herein after) is implemented.

FIG. 7 represents tumoroids (or spheroids) produced according to the present disclosure and obtained from MKN45 cells, 7 days after encapsulation in the modified dextran hydrogel microparticles according to the present disclosure and by using a 2.8% (w/v) gel obtained by using a Dextran-VS (DS=33%) (see example paragraph 1.2 below). The scale bar is 100 μm.

FIG. 8 represents four confocal images of human small intestinal apical-in (indicated by F-actin staining inside the organoid's crypt) organoid obtained according to example paragraph 3.2 below and stained by Doppl. The DNA-staining (DAPI, that is to say 4,6-diamidino-2-phenylindole), F-actin (named filamentous actin or actin filaments: Phalloidin was used to stain actin-F), MUC2 (a marker for goblet cells and mucus production), and SOX9 (a transcription factor associated with stem/progenitor cells in the intestinal epithelium) is shown respectively from the left to the right. Scale bar is 100 μm.

FIG. 9 represents a confocal image of human small intestinal apical-in (indicated by F-actin staining actin localized on the inner surface of the organoid crypts, indicating polarity) organoids obtained according to example paragraph 3.2 below and stained by Doppl. The DNA-staining (DAPI, that is to say 4′,6-diamidino-2-phenylindole) and F-actin (named filamentous actin or actin filaments: Phalloidin was used to stain actin-F) allows to estimate morphological structure of organoids. Scale bar is 100 μm.

FIG. 10 represents a confocal image of human small intestinal apical-out (indicated by F-actin localized on the outer surface of the organoid crypts, indicating polarity) organoids obtained according to example paragraph 3.3 and stained by Doppl. The DNA-staining (DAPI, that is to say 4′,6-diamidino-2-phenylindole) and F-actin (named filamentous actin or actin filaments: Phalloidin was used to stain actin-F) allows to estimate morphological structure of organoids. Scale bar is 100 μm.

FIG. 11 represents a graph which comprises curve of EC50 test. The duration of the test was 72 hours, R²=0.88 and EC50=119.2 nM. The curb was obtained on MKN45 spheroids in gel shells with CellTiter-GloR 2.0 Cell Viability Assay. The x-axis corresponds to MEKi (MEK inhibitor) log₁₀ (concentration), nM and the y-axis corresponds to Relative Cell Viability, the RLU (relative luminescence units) was normalized by negative control to obtain Relative Cell viability (see example paragraph 1.7 below).

FIG. 12 demonstrates tight spheroids produced in accordance with the present disclosure from H1975 cell line. Scale bar is 100 μM (see example paragraph 1.5bis below).

FIG. 13A represents two immunofluorescence images of whole mount hiPSC-derived spheroids at Day 7 post encapsulation. All nuclei are positive for both pluripotency markers (SOX2 and NANOG from the left to the right), proving the maintenance of the pluripotent state. Scale bar is 50 μm (see example paragraph 4 below).

FIG. 13B represents two immunofluorescence images which correspond to staining of alpha-Smooth muscle actin (α-SMA) indicating the majoritarian presence of mesodermal cells in the 3D cardiomyocyte spheroids at Day 21 and the DNA-staining (DAPI, that is to say 4, 6-diamidino-2-phenylindole) shows cells nucleus. Scale bar is 100 μm. (see example paragraph 4 below).

FIG. 13C is a graphical representation of the cardiomyocyte spheroids beating at Day 17 and Brightfield image of a cardiomyocyte spheroid at Day 17 that was used to record a video and quantify the beating frequency plotted below. Analysis was performed using MUSCLEMOTION (van Meer B. J. & Sala L. et al., Quantification of Muscle Contraction In Vitro and In Vivo Using MUSCLEMOTION Software: From Stem Cell-Derived Cardiomyocytes to Zebrafish and Human Hearts, Current Protocols in Human Genetics, 2018, doi: 10.1002/cphg.67) Scale bar is 100 μm. (see example paragraph 4 below).

FIG. 14A represents a schematic of the microfluidic chip designed for the reinjection of MKN45 spheroids encapsulated within a modified dextran hydrogel microparticle. Two key zones are highlighted: (1) the compaction zone, where initial spheroid alignment occurs, and (2) the chip outlet, where encapsulated spheroids exit the device. (see example paragraph 5 below).

FIG. 14B represents a visualization of MKN45 spheroids compacted at the chip's entry point, zone (1) of the chip presented on FIG. 14A. (see example paragraph 5 below).

FIG. 14C represents MKN45 spheroids encapsulated within a modified dextran hydrogel microparticle, zone (2) of the chip presented on FIG. 14A. (see example paragraph 5 below).

FIG. 15A represents a counter plot of the red fluorescence detection inside the hydrogel beads. This plot is based only with the beads with CHO cells inside (in this case the hydrogel beads with a positive green fluorescence, Calcein staining). The red fluorescence is proportional to the IgG secretion thanks to a secondary antibody anti-Fc labelled with a red fluorophore (Alexa 647). On this plot, it can be distinguished easily the CHO population that did not secret (“Secretion”, as written on the left of the plot) and the CHO population that has secreted IgG (“Secretion+”, as written on the right of the plot) (see example paragraph 2.5 below).

FIG. 15B represents a graph showing different IgG secretion measurements for different pools of CHO cells. Each point corresponds to two measures (x and y) of an IgG secretion estimation of a dedicated CHO cell pool. The IgG secretion is measured with two different manners. In the x axis, the value is measured in hydrogel beads with the mean intensity fluorescence (MFI) measured by the flow cytometer on the secreted cells (“Secretion+”, FIG. 15A). In the y axis, the value is measured, on the supernatant CHO culture, by biolayer interferometry on the Octet device that gave the direct value of the IgG secreted by a CHO cell. After plotting all the different experiments, a linear regression was calculated demonstrated a strong correlation between the two different measurement methods (R²=0,89). This result demonstrates that the MFI measurement gave a good estimation of the real productivity of CHO cells sample (see example paragraph 2.5 below).

FIG. 15C represents two counter plots measuring the IgG secretion of two CHO pools before and after sorting the higher secretor CHO cells. Before sorting the CHO cells secreting IgG represent less than 20% of the CHO population (left plot, “Secretion+”). After sorting only, the high IgG secretor CHO cells thanks to a FACS, the CHO cells secreting IgG represent this time 90% of the CHO population (right plot, “Secretion+”). This result demonstrates the capacity of the hydrogel beads technology in accordance of the present disclosure to be compatible with a FACS and to be able to enrich a dedicated cell population based on its secretion measurement (see example paragraph 2.5 below).

FIG. 16 represents eight images of spheroids presented in two rows comprising four images each. The first row demonstrates the process of cell polymerization in a tube collector, where cells sediment under gravity, leading to the subsequent degradation of hydrogel beads and reducing the size and yield of spheroids. The second row shows the outcome of cell centering within hydrogel beads, achieved through polymerization during their movement through a capillary wrapped around a Peltier element maintaining a temperature of 37° C. Scale bar is 50 μm (see example paragraph 6 below).

FIG. 17A represents the percentage of Relative cell viability with (see bar or column on the right of the graph named «in line polymerization») and without (see bar or column on the left of the graph named «standard process») in line polymerization. These results demonstrate a better viability of the cells when the in-line polymerization is used (see example paragraph 6 below).

FIG. 17B represents the percentage of relative Jurkat cell activation without hydrogel beads (see bar or column on the left of the graph), in hydrogel beads with (see bar or column on the right of the graph) and without (see bar or column in the middle of the graph) in line polymerization. These results demonstrate a higher clusterization (also named clustering in the present disclosure) of the membrane proteins in the context of in line polymerization compared to the fact to not use it (see example paragraph 6 below).

FIG. 18A represents hydrogel beads in accordance with the present disclosure which encapsulate yeast cells after the extraction from oil. Scale bar is 25 μm (see example paragraph 7 below).

FIG. 18B represents 6 hours of growth of yeast cells encapsulated in hydrogel beads in accordance with the present disclosure after the recuperation. Scale bar is 25 μm (see example paragraph 7 below).

FIG. 18C represents 12 hours of growth of yeast cells encapsulated in hydrogel beads in accordance with the present disclosure after the recuperation. Scale bar is 25 μm (see example paragraph 7 below).

In the present disclosure and in particular in the figures, well-known scientific notations comprising the letter “e” are used. For example, “1e7” means 10⁷, “2.5e7” means 2.5*10⁷, “1e2” means 100 and “5e6” means 5*10⁶.

### DETAILED DESCRIPTION OF THE DISCLOSURE

As apparent in more details in the illustrative part of the present disclosure, the hydrogel microparticles or hydrogel microbeads according to the present disclosure may be manufactured in high yields and easily manipulated for further use, for example by subsequent pipetting step, centrifugation step or distribution in well plates.

As detailed hereinbelow, depending on the variation of concentration of hydrogel and linker, hydrogel microbeads with various physical properties may be obtained. They may be very hard or very soft and this thus gives the possibility to adapt the stiffness of the hydrogel easily to the type of considered cells for encapsulation.

Compared to known hydrogel microparticles or hydrogel microbeads, the hydrogel microparticles or hydrogel microbeads according to the present disclosure may be obtained easily with high yields in a large variation of droplet generating devices. In particular, the hydrogel microparticles or hydrogel microbeads according to the present disclosure could be biocompatible, accessible for standard laboratory manipulation technics, as pipetting, centrifugation, distribution in well plates, as well as more complex analysis methods like FACS (Fluorescence-activated cell sorting), immunofluorescence (IF) analysis (immunostaining), as well as secretome and transcriptomics analysis (RNA extraction, RNA sequencing). The hydrogel microparticles or hydrogel microbeads according to the present disclosure have a very good transparency in particular for imaging technics, and can be dissolved in a biocompatible manner so as to access microbead load. Advantageously, they enable prolongated cell proliferation, under the mild conditions, without important structure changes, can allow protein and growth factor diffusion and can be produced keeping low coefficient of size variation.

Advantageously, as illustrated in the examples that follow, even for cells known to form only loose aggregates, by using the technology described in the present disclosure, tight tumoroids were obtained from the IGROV1 cells (human ovarian carcinoma cells), MKN45 cells (poorly differentiated adenocarcinoma of the stomach cells), or H1975 (human non-small cell lung carcinoma cells) which could be manipulated without damaging them, as they were protected by hydrogel shell (see in particular example 1.2, example 1.4, and example 1.5 bis). This advantage is also shown in the experimental part for patient-derived stem cells (see in particular example 3), for human induced pluripotent stem cells (see in particular example 4), and for yeast cells (see in particular example 7).

A further advantage, as illustrated in the examples that follow, is that the hydrogel microparticles or hydrogel microbeads according to the present disclosure allow to produce a wide variety of spheroids which exhibit a high level of manipulability as mentioned above, in particular which are obtained in a greater quantity than would be obtained for example by using Ultra-low attachment (ULA) plate, and which are thus useful for universal applications (see in particular example 1.5). Thus, the hydrogel microparticles or hydrogel microbeads according to the present disclosure is found to be particularly advantageous for offering a wide range of microbeads with respect to stiffness, with a reproducible and simple process.

A further advantage, as illustrated in the examples that follow, is that, spheroids that may be obtained according to the process according to the present disclosure may directly be subject to further experimentation, such as lysis of spheroids, antibody (for instance immunoglobulins such as IgG) and small molecules drug testing (screenings), RNA sequencing, RNA extraction, and immune staining decreasing the number of required manipulations (see in particular examples 1.7, 1.9, 1.10 and 2). In particular, as illustrated in example 2 that follows, spheroids that may be obtained according to the process according to the present disclosure may be used in a method to select the best IgG producer cells.

Another advantage, as illustrated in the examples below is that, after bead jellification and demulsification, cell media or other immersion solutions could be changed, which allows a precise control over cell biology and post encapsulation manipulations.

A further advantage is that cells, spheroids, and other cell aggregates, encapsulated in the hydrogel microbeads could be used for automated and robotized pipelines, because of the high manipulability of the hydrogel microbeads and protection function of them on encapsulated objects.

Another advantage is that jellification of the microbeads could be performed in a wide temperature range, with no need to manipulate at particular temperature conditions, but temperature can be used to change the speed of jellification.

A further advantage is the possibility of using PDSCs (patient-derived stem cells) for high-throughput organoid production, by using the technology described in present disclosure, as well as the ease of controlling their polarity due to a spheroid production protocol that does not require re-optimization when the hydrogel composition is modified (see example 3). The apical-out organoids enable also the direct evaluation of the toxicity of the drug on the intestinal lumen. In addition, the use of dextran-based hydrogel according to the present disclosure significantly reduces the amount of animal-derived extracellular matrix required for spheroid production, which enables the use of high-throughput droplet microfluidics methods, as there is no significant increase in the viscosity of the discrete phase.

Physiologically structured organoids that are produced preserve native tissue architecture, making them suitable for any studies requiring physiological tissue morphology.

Non-physiologically structured organoids present promising models for advanced drug testing or direct physical constant measurement on specific tissues of an organ usually non-directly accessible from the physiological tissue morphology. Apical-out organoids as described in example 3 enables the direct evaluation of the effects of a drug on the intestinal lumen such as its activity, toxicity as non-limited examples or enables measurement of the diffusion of a compound/drug simulating peroral drug administration.

Another advantage, as illustrated in the examples that follow, is the ability to grow spheroids from hiPSCs (human induced pluripotent stem cells), which retain their pluripotency, in hydrogel beads as defined in present disclosure, thereby enabling the implementation of any differentiation protocol. For instance, differentiation into cardiomyocytes, that shows beating activity, was demonstrated (see example 4). Thus, the hydrogel microbeads as defined in the present disclosure support cell viability, adherence, and the formation of embryoid bodies, while maintaining pluripotency.

A further advantage, as illustrated in the examples below, is that the hydrogel microparticles or hydrogel microbeads according to the present disclosure, allow spheroid reinjections into a compartment, more particularly MKN45 spheroid reinjections, while avoiding mechanical dissociation of the spheroids, facilitating smooth transit through the microfluidic channel or circuit, preventing clogging, mitigating shear stress during droplet formation when for example encapsulated into a droplet and ensuring more controlled and efficient spheroid encapsulation (see example 5).

Besides, several approaches have already been proposed to address the problems of cell escape, and more generally of cell centering. For example, the following off-chip methods and on-chip methods which are well-known in the art: Off-chip methods:
When growing cells in Matrigel beads, adding 1% Matrigel to the cell culture medium can reduce cell escape by preventing bead dissolution, thereby prolonging the retention of cells within the beads.
Hydrogel bead emulsions containing cells can also be polymerized while spinning at 1000 rpm on an orbital shaker to enhance cell centering.

On-chip methods:
Beads with cells can be re-encapsulated within another hydrogel bead, either made of the same material or another non-degradable material.
Additionally, a method involving the slow polymerization of Dextran-TA/HRP droplets has been proposed. This is achieved by moving the droplets through a long PDMS channel, where hydrogen peroxide gradually diffuses through the channel walls to allow prolongated polymerization ensuring cells centering in the beads.

And in this regard, another advantage, as illustrated in the examples below, is that the hydrogel microparticles or hydrogel microbeads according to the present disclosure, allow cell centering (see example 6). The cell centering within beads enables the formation of larger cells as defined in the present disclosure such as spheroids or organoids using smaller droplets, thanks to the uniform distribution of gel around the cells and this allows to avoid cell from rupturing the gels at early stages of 3D cell structure development and increase overall yield of 3D cell models production.

Another advantage that cell centering allows to avoid cells escape from the bead during mechanical manipulation like pipetting, centrifugation of FACS sorting improving overall yield of the manipulation process.

In the present disclosure, the inventors aim to propose a new method for addressing the challenge of cell centering. This approach leverages the accelerated reaction between vinyl sulfone and thiol upon heating. In addition, the heating during cell centering increases the rate of polymerization and the gel is more porous which enables clustering of transmembrane proteins such as CD3 receptors.

Other features, alternative forms, and advantages of the hydrogel microparticles, processes for manufacturing them and associated uses and methods according to the disclosure will emerge more clearly on reading the description and the examples that follow, which are given as non-limiting illustrations of the disclosure.

The expressions “between . . . and . . . ”, “ranging from . . . to . . . ”, “formed from . . . to . . . ”, and “varying from . . . to . . . ” should be understood as being meaning inclusive of limits, unless otherwise stated.

In the description and the examples, percentages are percentages by weight unless otherwise stated. Percentages are thus expressed as weights relative to the total weight of the composition.

### Use of Modified Dextran Hydrogel Microparticles for Encapsulating Cell(S).

As mentioned above, herein is provided the use of modified dextran hydrogel microparticles comprising (i) at least one vinyl sulfone functionalized dextran, and (ii) at least one crosslinkable polymer having at least two thiol functions, wherein
the dextran has a molecular weight comprised between 5 and 500 kDa, and
the substitution degree of the dextran by the vinyl sulfone is comprised between 5 and 60%,
   for encapsulating at least one synthetic or natural cell.

According to one embodiment, the use of modified dextran hydrogel microparticles as defined in the present disclosure is aimed at cultivating said at least one synthetic or natural cell, for example as a 3D cell culture, in particular by incubating the microparticles in an environment suitable to allow for cell survival, cell growth, cell differentiation and/or cell proliferation such as clonal expansion, more particularly for producing a cell cluster, a spheroid, an organoid, a gastruloid, a tumoroid or a tissue.

According to one embodiment, the use of modified dextran hydrogel microparticles as defined in the present disclosure is aimed at screening or discriminating compounds, proteins, polypeptides, oligopeptides, nucleic acids, cell secretome or antibodies, in particular by performing RNA-sequencing, by using cell staining such as chemical or immunostaining or by using imaging such as electronic imaging or optic imaging.

Herein is also provided a process for encapsulating at least one synthetic or natural cell comprising at least a step of encapsulating said at least one synthetic or natural cell in a modified dextran hydrogel microparticles comprising (i) at least one vinyl sulfone functionalized dextran, and (ii) at least one crosslinkable polymer having at least two thiol functions, wherein
the dextran has a molecular weight comprised between 5 and 500 kDa, and
the substitution degree of the dextran by the vinyl sulfone is comprised between 5 and 60%.

### Modified Dextran Hydrogel Microparticles

Also provided herein are modified dextran hydrogel microparticles comprising (i) at least one vinyl sulfone functionalized dextran, (ii) at least one crosslinkable polymer having at least two thiol functions and (iii) at least one synthetic or natural cell, wherein
the dextran has a molecular weight comprised between 5 and 500 kDa, and
the substitution degree of the dextran by the vinyl sulfone is comprised between 5 and 60%,
provided that the at least one crosslinkable polymer having at least two thiol functions is different from a crosslinkable polymer comprising dextran having a molecular weight comprised between 30 and 50 kDa as the polymer chain.

Also provided herein are modified dextran hydrogel microparticles comprising (i) at least one vinyl sulfone functionalized dextran, (ii) at least one crosslinkable polymer having at least two thiol functions and (iii) at least one synthetic or natural cell,
wherein
   the dextran has a molecular weight comprised between 5 and 500 kDa,
   the substitution degree of the dextran by the vinyl sulfone is comprised between 5 and 60%, and
   the crosslinkable polymer comprises
      at least a polymer chain selected from a group consisting of PEG, gelatin, agarose, alginate, hyaluronic acid, collagen, proteins, polyacrylamide, block-polymers,
      at least an (C4-C12)alkylene chain, optionally substituted by one or two hydroxy group(s), or
      their mixtures,
   in particular PEG.

According to one embodiment, the size of the modified dextran hydrogel microparticles as defined in the present disclosure is between 1 μm and 1000 μm, in particular between 1 μm and 999 μm, in particular between 10 μm and 500 μm, in particular between 30 μm and 180 μm.

According to another embodiment, the modified dextran hydrogel microparticles has a coefficient of variation in size lower than 50%, in particular lower than 20%, and more particularly lower than 5%.

In one embodiment, the modified dextran hydrogel microparticles are monodisperse modified dextran hydrogel microparticles.

In the context of the disclosure, by monodisperse is meant that the modified dextran hydrogel microparticles has a coefficient of variation in size lower than 20%, in particular lower than 10%, more particularly lower than 5%.

### Vinyl Sulfone Functionalized Dextran

As explained above, the modified dextran hydrogel microparticles according to the present disclosure comprise (i) at least one vinyl sulfone functionalized dextran.

According to a particular embodiment, the modified dextran hydrogel microparticles according to the present disclosure comprise one vinyl sulfone functionalized dextran as defined in the present disclosure.

According to another particular embodiment, the modified dextran hydrogel microparticles according to the present disclosure comprise more than one vinyl sulfone functionalized dextrans as defined in the present disclosure.

According to another specific embodiment, the modified dextran hydrogel microparticles described in the present disclosure include various substitution degrees of dextran by vinyl sulfone, such as 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40%.

When several vinyl sulfone functionalized dextrans as defined in the present disclosure are comprised in the modified dextran hydrogel microparticles according to the present disclosure, they can be identical or different, in particular in terms of molecular weight and/or substitution degree.

The vinyl sulfone functionalized dextran as defined in the present disclosure is a hydroxyl-containing water-soluble polymer, more particularly a dextran which has been functionalized by vinyl sulfone groups via a one-step reaction method. This method, named one-step “click” method is well described in Yu et al. (One-step “Click” method for generating vinyl sulfone groups on hydroxyl-containing water-soluble polymers”, Biomacromolecules, 2012, 13, 937-942, America Chemical Society). For example, the dextran can be dissolved in a base such as sodium hydroxide (NaOH) and then modified by the adding of divinyl sulfone (DVS). Furthermore, thanks to this simple “click” chemistry method, the degree of modification (also named substitution degree in the present disclosure) of the dextran can be controlled by varying the pH, reaction time, and DVS to molar ratio. This modified dextran, instead of forming a pre-cross-linked hydrogel, generates a “clickable” precursor which can, for example, react with thiol functions via a thiol-Mickael “click” reaction making them excellent candidates to prepare in situ hydrogels.

According to a particular embodiment, the vinyl sulfone functionalized dextran as defined in the present disclosure does not bear carboxylate groups. In other terms, the vinyl sulfone functionalized dextran as defined in the present disclosure is devoid or is free of carboxylate groups. In particular, the vinyl sulfone functionalized dextran as defined in the present disclosure is not first carboxymethylated.

By carboxylate groups, in the sense of the present disclosure, it can be mentioned, for example, a group of formula (A)

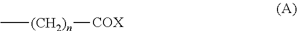

wherein
n is 1, 2, 3, 4, 5, 6 or 7; and
X represents —OH, —ONa, —OK or a-(R)ₘG group in which m is 0 or 1, R is a linear or branched alkyl divalent radical comprising from 1 to 6 carbon atoms and optionally one or more heteroatoms such as oxygen, nitrogen or sulfur, and G is a linear or branched or cyclic alkyl divalent radical comprising from 1 to 6 carbon atoms and may comprise one or more heteroatoms such as oxygen, nitrogen or sulfur.

In addition, the vinyl sulfone functionalized dextran as defined in the present disclosure is not a crosslinked dextran polymer.

Herein is also provided a process for preparing the vinyl sulfone functionalized dextran as defined in the present disclosure, wherein it comprises at least the following steps of:
(i) Dissolving a dextran having a molecular weight comprised between 5 and 500 kDa, in particular between 40 and 150 kDa, more particularly between 50 and 100 kDa, for instance of 70 kDa in an inorganic base such as sodium hydroxide:
(ii) Adding divinyl sulfone in an amount of from 1 to 2 equivalents, in particular 1.5 equivalents, compared to hydroxyl groups of the dextran, and reacting from 30 s to 30 min, in particular from 1 min to 5 min.
(iii) Adjusting the pH to a value comprised between 3 to 6, particularly of 5 with an acid, for instance with hydrochloric acid:
(iv) Optionally purifying the thus obtained modified dextran, for instance by dialysis to obtain a dialyzed solution; and
(v) Optionally filtering and freeze-drying the dialyzed solution.

As described above, the molecular weight of the dextran used in the vinyl sulfone functionalized dextran as defined in the present disclosure is comprised between 5 and 500 kDa and the substitution degree of the dextran by the vinyl sulfone is comprised between 5 and 60%.

According to a particular embodiment, the molecular weight of the dextran is comprised between 40 and 150 kDa, in particular between 50 and 100 kDa, for example is 70 kDa.

According to another particular embodiment, the substitution degree of the dextran by the vinyl sulfone is comprised between 10 and 40%, in particular between 18 and 38%.

According to a more preferred embodiment, the substitution degree of the dextran by the vinyl sulfone is comprised between 20 and 35%, in particular between 21 and 34%, for instance between 22 and 23% or between 32 and 34%.

### Crosslinkable Polymer Having at Least Two Thiol Functions

As explained above, the modified dextran hydrogel microparticles according to the present disclosure further comprise (ii) at least one crosslinkable polymer having at least two thiol functions.

According to a particular embodiment, the modified dextran hydrogel microparticles according to the present disclosure comprise one crosslinkable polymer having at least two thiol functions as defined in the present disclosure.

According to another particular embodiment, the modified dextran hydrogel microparticles according to the present disclosure comprise more than one crosslinkable polymer having at least two thiol functions as defined in the present disclosure.

According to another particular embodiment, the modified dextran hydrogel microparticles according to the present disclosure comprise for example, two, three, four, five, six, seven, eight, nine, or ten crosslinkable polymers having at least two thiol functions as defined in the present disclosure.

The crosslinkable polymers according to the present disclosure can have a molecular weight ranging from 150 Da to 200 kDa, particularly from 1 kDa to 50 kDa, more particularly from 1 kDa to 10 kDa.

For example, when the crosslinkable polymer according to the present disclosure comprises a polymer chain which is PEG, the PEG can have a molecular weight ranging from 150 Da to 200 kDa, particularly from 1 kDa to 50 kDa, more particularly from 1 kDa to 10 kDa, for instance 1.5 kDa.

Needless to say that the skilled person would adapt the molecular weight of the crosslinkable polymers according to the present disclosure depending on the desired application.

When several crosslinkable polymers having at least two thiol functions as defined in the present disclosure are comprised in the modified dextran hydrogel microparticles according to the present disclosure, they can be identical or different, in particular in terms of nature, molecular weight and/or number of thiol functions.

According to one embodiment, the crosslinkable polymer as defined in the present disclosure comprises
at least a polymer chain selected from a group consisting of PEG, dextran, gelatin, agarose, alginate, hyaluronic acid, collagen, proteins, polyacrylamide, block-polymers,
at least an (C4-C12)alkylene chain, optionally substituted by one or two hydroxy group(s), or
their mixtures,
in particular PEG.

According to a preferred embodiment, the crosslinkable polymer as defined in the present disclosure comprises at least a polymer chain which is PEG.

According to a particular embodiment, the crosslinkable polymer as defined in the present disclosure is selected from a group consisting in a 2 to 12 arms polyethylene glycol thiol, dithiothreitol (also named DTT), dextran-SH, gelatin-SH, collagen-SH, proteins having at least two thiol functions and block-polymers having at least two thiol functions, in particular a PEG dithiol (also named PEGDT, poly(ethylene glycol)dithiol, thiol-PEG-thiol, PEG-2-SH, SH-PEG-SH) or a four-arm PEG thiol (also named a four-arm polyethylene glycol thiol, a four-arm PEG SH, a four-arm polyethylene glycol SH).

According to a preferred embodiment, the crosslinkable polymer as defined in the present disclosure is PEG dithiol.

According to a particular embodiment, the weight ratio of vinyl sulfone functionalized dextran and crosslinkable polymer having at least two thiol functions is comprised between 1% and 30%, in particular between 2% and 20%, particularly between 2.5% and 10%.

### Synthetic or Natural Cell(s)

As explained above, the modified dextran hydrogel microparticles in accordance with the present disclosure are used to encapsulate at least one synthetic or natural cell.

The synthetic or natural cell to be encapsulated as defined in the present disclosure can be prepared according to any method known by the skilled person.

For example, a process for the preparation of said synthetic or natural cell to be encapsulated can comprise the following steps:
1. Taking the flask with the cells
2. Keeping the supernatant
3. Adding an appropriate volume, such as 1 mL, of cell detachment solution or proteolytic and collagenolytic enzymes, such as accutase solution to cell culture flask such as cell culture flask T25, and another volume such as 2 mL to cell culture flask such as cell culture flask T75
4. Keeping a suitable time such as 10-15 min until the detachment of the cell at a temperature ranging from 20° C. to 45° C. such as 37° C.
5. Adding an appropriate volume such as 5*volume of accutase fresh cell medium containing a growth supplement such as FBS (fetal bovine serum) and transfer cells solution to the kept supernatant
6. Centrifuging for example at 300 g for 5 min:
7. Removing the supernatant.
8. Diluting cells at desired concentration in encapsulation buffer
9. Filtering them through a strainer, for example a 37 μm strainer; and
10. Using immediately for example for the preparation of encapsulation solution.

According to another example, a process for the preparation of said synthetic or natural cell to be encapsulated can comprise the following steps:
1. Taking the flask with the cells
2. Removing the supernatant
3. Rinsing with an appropriate volume of an isotonic solution such as at least 5 mL of PBS (Phosphate buffered saline) for cell culture Flask such as cell culture flask T25-T75
4.Adding an appropriate volume, such as 1 mL, of a proteolytic enzyme such as trypsin to cell culture flask such as cell culture flask T25, and another volume such as 2 mL to cell culture flask such as cell culture flask T75
5. Keeping a suitable time such as 10-15 min until the detachment of the cell at a temperature ranging from 20° C. to 45° C. such as 37° C.
6. Adding fresh cell medium containing FBS, an appropriate volume such as 5*volume of trypsin used and transfer cells solution to the centrifuge tube
7. Centrifuging for example at 300 g for 5 min;
8. Removing the supernatant.
9. Diluting cells at desired concentration in encapsulation buffer
10. Filtering them through a strainer, for example a 37 μm strainer; and
11. Using immediately for example for the preparation of encapsulation solution.

According to one particular embodiment, the modified dextran hydrogel microparticles in accordance with the present disclosure are used to encapsulate one single synthetic or natural cell.

According to another particular embodiment, the modified dextran hydrogel microparticles in accordance with the present disclosure are used to encapsulate more than one synthetic or natural cells.

According to another particular embodiment, the modified dextran hydrogel microparticles in accordance with the present disclosure are used to encapsulate two, three, four, five, six, seven, eight, nine or ten, synthetic or natural cells, in other terms until the volumetric saturation of the compartment is obtained.

The cell in accordance with the present disclosure can be a plurality of cells of the same or different cell types and/or cell subtypes.

At the end of the process of encapsulation, some modified dextran hydrogel microparticles in accordance with the present disclosure can be empty, that is to say they do not comprise at least one synthetic or natural cell as defined in the present disclosure.

According to one embodiment, the synthetic or natural cell is selected from eukaryotic cells such as a mammalian cells, for instance human cells, prokaryotic cells such as bacterial cells or archaea cells, cancerous cells, tumoral cells, somatic cells, spleen cells, stem cells, progenitor cells, precursor cells, fully differentiated cells, undifferentiated cells, germ cells, cells of unicellular organism or of multi-cellular organism, fungal cells, splenocytes and hybridoma cell.

According to a particular embodiment, the synthetic or natural cell is selected from the group consisting of connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof.

According to a particular embodiment, the synthetic or natural cell is selected from the group consisting of gastric cancer cell, ovarian carcinoma cell, ovary cell, hepatoma cell, breast adenocarcinoma cell, lung adenocarcinoma cell such as non-small lung adenocarcinoma cell, yeast cell, induced pluripotent stem cell (IPSc), and patient-derived stem cell (PDSC).

According to a preferred embodiment, the synthetic or natural cell is selected from the group consisting of human gastric cancer cell, human ovarian carcinoma cell, hamster ovary cell, human hepatoma cell, human breast adenocarcinoma cell, human non-small lung carcinoma cell, yeast cell, human induced pluripotent stem cell (hiPSc), and patient-derived stem cell.

According to a more preferred embodiment, the synthetic or natural cell is selected from the group consisting of MKN45 cell, IGROV1 cell, CHO cell, HEP3B cell, MDA-MB-468 cell, H1975 cell, yeast cell, human induced pluripotent stem cell, and patient-derived stem cell.

According to another more preferred embodiment, the synthetic or natural cell is MKN45 cell.

According to another more preferred embodiment, the synthetic or natural cell is IGROV1 cell.

According to another more preferred embodiment, the synthetic or natural cell is CHO cell.

According to another more preferred embodiment, the synthetic or natural cell is HEP3B cell.

According to another more preferred embodiment, the synthetic or natural cell is MDA-MB-468 cell.

According to another more preferred embodiment, the synthetic or natural cell is H1975 cell.

According to another more preferred embodiment, the synthetic or natural cell is yeast cell.

According to another more preferred embodiment, the synthetic or natural cell is human induced pluripotent stem cell.

According to another more preferred embodiment, the synthetic or natural cell is patient-derived stem cell.

Herein are also provided modified dextran hydrogel microparticles comprising (i) at least one vinyl sulfone functionalized dextran as defined in the present disclosure, (ii) at least one crosslinkable polymer having at least two thiol functions as defined in the present disclosure and (iii) at least one synthetic or natural cell as defined in the present disclosure obtainable, by a process as defined in the present disclosure.

According to a preferred embodiment, the disclosure relates to modified dextran hydrogel microparticles comprising (i) at least one vinyl sulfone functionalized dextran as defined in the present disclosure, (ii) at least PEG dithiol as defined in the present disclosure and (iii) at least one synthetic or natural cell as defined in the present disclosure, obtainable by a process as defined in the present disclosure.

According to a particularly preferred embodiment, the disclosure relates to modified dextran hydrogel microparticles comprising (i) at least one vinyl sulfone functionalized dextran as defined in the present disclosure, (ii) at least PEG dithiol as defined in the present disclosure and (iii) at least one synthetic or natural cell selected from the group consisting in human gastric cancer cell line derived from poorly differentiated gastric adenocarcinoma such as MKN-45 cell line, human ovarian carcinoma cell line such as IGROV-1 cell line, Chinese hamster ovary cell line such as CHO cell line, human hepatoma cell line such as HEP3B cell line, human breast adenocarcinoma cell line such as MDA-MB-468 cell line, human non-small cell lung carcinoma cell line such as H1975 cell line, yeast cell line, human induced pluripotent stem cell line, and patient-derived stem cell line obtainable by a process as defined in the present disclosure.

According to a more preferred embodiment, the disclosure relates to modified dextran hydrogel microparticles comprising (i) at least one vinyl sulfone functionalized dextran as defined in the present disclosure, (ii) at least PEG dithiol as defined in the present disclosure and (iii) at least one human gastric cancer cell line derived from poorly differentiated gastric adenocarcinoma such as MKN-45 cell line, obtainable by a process as defined in the present disclosure.

According to another more preferred embodiment, the disclosure relates to modified dextran hydrogel microparticles comprising (i) at least one vinyl sulfone functionalized dextran as defined in the present disclosure, (ii) at least PEG dithiol as defined in the present disclosure and (iii) at least one human ovarian carcinoma cell line such as IGROV-1 cell line, obtainable by a process as defined in the present disclosure.

According to another more preferred embodiment, the disclosure relates to modified dextran hydrogel microparticles comprising (i) at least one vinyl sulfone functionalized dextran as defined in the present disclosure, (ii) at least PEG dithiol as defined in the present disclosure and (iii) at least one Chinese hamster ovary cell line such as CHO cell line, obtainable by a process as defined in the present disclosure.

According to another more preferred embodiment, the disclosure relates to modified dextran hydrogel microparticles comprising (i) at least one vinyl sulfone functionalized dextran as defined in the present disclosure, (ii) at least PEG dithiol as defined in the present disclosure and (iii) at least one human hepatoma cell line such as HEP3B cell line, obtainable by a process as defined in the present disclosure.

According to another more preferred embodiment, the disclosure relates to modified dextran hydrogel microparticles comprising (i) at least one vinyl sulfone functionalized dextran as defined in the present disclosure, (ii) at least PEG dithiol as defined in the present disclosure and (iii) at least one human breast adenocarcinoma cell line such as MDA-MB-468 cell line, obtainable by a process as defined in the present disclosure.

According to another more preferred embodiment, the disclosure relates to modified dextran hydrogel microparticles comprising (i) at least one vinyl sulfone functionalized dextran as defined in the present disclosure, (ii) at least PEG dithiol as defined in the present disclosure and (iii) at least one human non-small cell lung carcinoma cell line such as H1975 cell line, obtainable by a process as defined in the present disclosure.

According to another more preferred embodiment, the disclosure relates to modified dextran hydrogel microparticles comprising (i) at least one vinyl sulfone functionalized dextran as defined in the present disclosure, (ii) at least PEG dithiol as defined in the present disclosure and (iii) at least one yeast cell line, obtainable by a process as defined in the present disclosure.

According to another more preferred embodiment, the disclosure relates to modified dextran hydrogel microparticles comprising (i) at least one vinyl sulfone functionalized dextran as defined in the present disclosure, (ii) at least PEG dithiol as defined in the present disclosure and (iii) at least one human induced pluripotent stem cell (hiPSc) line, obtainable by a process as defined in the present disclosure.

According to another more preferred embodiment, the disclosure relates to modified dextran hydrogel microparticles comprising (i) at least one vinyl sulfone functionalized dextran as defined in the present disclosure, (ii) at least PEG dithiol as defined in the present disclosure and (iii) at least one patient-derived stem cell line, obtainable by a process as defined in the present disclosure.

According to another particular embodiment, the disclosure relates to modified dextran hydrogel microparticles according to the present disclosure, wherein a bulk modified dextran hydrogel formed by reacting the at least one vinyl sulfone functionalized dextran as defined in the present disclosure, and the at least one crosslinkable polymer having at least two thiol functions as defined in the present disclosure in the same weight ratio as the one used for forming said microparticles, presents a Young's modulus (elastic modulus) comprised between 10 and 50 kPa, in particular from 20 to 30 kPa: more particularly from 20 to 25 kPa.

According to another particular embodiment, the disclosure relates to modified dextran hydrogel microparticles according to the present disclosure, wherein a bulk modified dextran hydrogel formed by reacting the at least one vinyl sulfone functionalized dextran as defined in the present disclosure, and the at least one crosslinkable polymer having at least two thiol functions as defined in the present disclosure in the same weight ratio as the one used for forming said microparticles, presents a shear modulus comprised between 3.33 and 16.66 kPa, in particular from 6.66 to 10 kPa: more particularly from 6.66 to 8.33 kPa.

According to a preferred embodiment, the disclosure relates to modified dextran hydrogel microparticles according to the present disclosure, wherein a bulk modified dextran hydrogel formed by reacting the at least one vinyl sulfone functionalized dextran as defined in the present disclosure, and the PEG dithiol as defined in the present disclosure in the same weight ratio as the one used for forming said microparticles, presents a Young's modulus (elastic modulus) comprised between 10 and 50 kPa, in particular from 20 to 30 kPa: more particularly from 20 to 25 kPa.

According to a preferred embodiment, the disclosure relates to modified dextran hydrogel microparticles according to the present disclosure, wherein a bulk modified dextran hydrogel formed by reacting the at least one vinyl sulfone functionalized dextran as defined in the present disclosure, and the PEG dithiol as defined in the present disclosure in the same weight ratio as the one used for forming said microparticles, presents a shear modulus comprised between 3.33 and 16.66 kPa, in particular from 6.66 to 10 kPa: more particularly from 6.66 to 8.33 kPa.

According to another particular embodiment, the disclosure relates to modified dextran hydrogel microparticles according to the present disclosure, wherein it presents a porosity up to 200 kDa less than 700 kDa, as quantified by analyzing the diffusion of fluorescently labeled molecules of various molecular weights through the hydrogel matrix. On this aspect, the inventors stated that high porosity of the hydrogel microbeads, even at high concentration of hydrogel, allow to simply mark the load of the beads with antibodies, as well as directly test therapeutic antibodies on spheroids grown in beads.

### Process for Preparing Modified Dextran Hydrogel Microparticles

Also provided herein is a process for preparing modified dextran hydrogel microparticles according to the present disclosure, wherein it comprises at least a step of using a microfluidic device, in particular comprising at least two inlet channels that converge into a droplet generation region.

Microfluidic technology is a well-known technology which is widely used to prepare hydrogel microparticles (also named HMPs). Microfluidic emulsions are also compatible with the encapsulation of cells, and it allows to foretell and control the number of cells incorporated in a single HMP.

The microfluidic device suitable for the present disclosure can be any suitable microfluidic device well known by the skilled person.

According to a particular embodiment, the microfluidic device comprises at least two inlet channels that converge into a droplet generation region.

According to another particular embodiment, the microfluidic device comprises at least three inlet channels that converge into a droplet generation region. According to another particular embodiment, the microfluidic device comprises at least four inlet channels that converge into a droplet generation region.

According to another particular embodiment, the microfluidic device comprises at least five inlet channels that converge into a droplet generation region.

According to another particular embodiment, the microfluidic device comprises at least six inlet channels that converge into a droplet generation region.

According to a particular embodiment, the process as defined in the present disclosure comprises at least the following steps of:
(a) Providing a first solution which comprises the at least one synthetic or natural cell, in an encapsulation buffer and at least one vinyl sulfone functionalized dextran:
(b) Providing a second solution which comprises an encapsulation buffer, identical or different from the buffer used in step (a), and the at least one crosslinkable polymer having at least two thiol functions:
(c) Forming water-in-oil droplets by encapsulating in co-flow through the two inlet channels the first and second solutions of steps (a) and (b) and an oily phase:
(d) Polymerizing the water-in-oil droplets at a temperature comprised between 0.01 and 99° C., in particular between 1 and 50° C., for instance at 4° C., room temperature or 37° C. to obtain the modified dextran hydrogel microparticles in oil:
(d′) Optionally implementing during said step (d) a capillary tube at a chip outlet, said tube being long enough to allow a polymerization, in particular a total or a partial polymerization of the water-in-oil droplets, and said tube being in contact with a heating element (solid, liquid or gaseous), in particular said tube being wrapped around a heating element or in particular said tube being immerged in a water bath, at a temperature comprised between 0.01 and 99° C., in particular between 1 and 50° C., for instance at 4° C., room temperature or 37° C.; and
(e) Optionally extracting the modified dextran hydrogel microparticles from the oily phase by washing, extracting, demulsification, electric fields destabilization, chemical destabilization and/or oil evaporation, for obtaining modified dextran hydrogel microparticles in an aqueous solution.

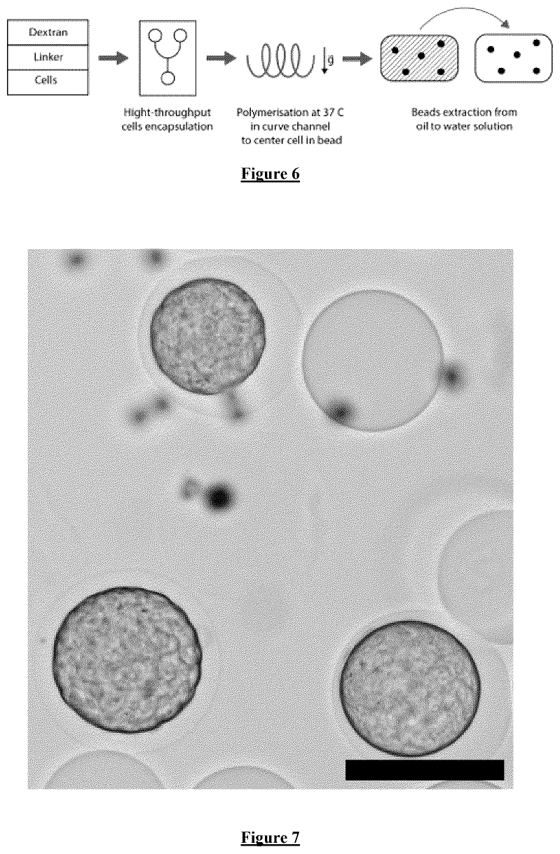

FIG. 6 provides a schematic illustration of one embodiment of the process according to the present disclosure, wherein an extraction step (step (e) as detailed herein after) is implemented.

### Step (a)

As mentioned above, the process in accordance with the present disclosure comprises the step (a).

The first solution provided in step (a) comprises the at least one synthetic or natural cell as defined in the present disclosure, in an encapsulation buffer and at least one vinyl sulfone functionalized dextran as defined in the present disclosure.

According to a particular embodiment, the encapsulation buffer used in step (a) is selected from the group consisting in any biocompatible cell media, adjusted or not to a particular pH, containing or not containing fetal serum (bovine, calf, horse, pig, sheep, or goat), biocompatible buffer solutions, for example PBS and DPBS (Dulbecco Phosphate Buffered Saline) with or without supplementary salts, protein mix or surfactant, as well as any cell-free buffers and solutions, and mixtures thereof.

The encapsulation buffer used in step (a) is any suitable cell media well-known by the skilled person for a given cell type.

According to one embodiment, the encapsulation buffer used in step (a) is selected from the group consisting in any biocompatible cell media.

According to a preferred embodiment, the encapsulation buffer used in step (a) is RPMI-1640 (Roswell Park Memorial Institute medium-1640) medium supplemented with GlutaMAX™, HEPES (4-(2-hydroxyethyl)-1-piperazineethanesulfonic acid) and Fetal Bovine serum (FBS).

According to another preferred embodiment, the encapsulation buffer used in step (a) is RPMI-1640 (Roswell Park Memorial Institute medium-1640) medium supplemented with GlutaMAX™, HEPES and Fetal Bovine serum.

The step (a) can be performed at a temperature comprised between 0° C. and 99° C., in particular at room temperature (25° C.).

The cells used in the process according to the present disclosure may have been stored at extremely low temperatures, typically in liquid nitrogen or a −80° C. freezer, to preserve their viability and functionality for long-term storage so that a preliminary step of defrosting cells may be necessary prior to their use into the process of the disclosure, as well as necessitate one or several passages prior to their use.

### Step (b)

As mentioned above, the process in accordance with the present disclosure comprises the step (b).

The second solution provided in step (b) comprises an encapsulation buffer, identical or different from the buffer used in step (a), and the at least one crosslinkable polymer having at least two thiol functions as defined in the present disclosure.

According to a particular embodiment, the encapsulation buffer used in step (b) is selected from the group consisting in any biocompatible cell media, adjusted or not to a particular pH, containing or not containing fetal serum (bovine, calf, horse, pig, sheep, or goat), biocompatible buffer solutions, for example PBS and DPBS (Dulbecco Phosphate Buffered Saline) with or without supplementary salts, protein mix or surfactant, as well as any cell-free buffers and solutions, and mixtures thereof.

The encapsulation buffer used in step (b) is any suitable cell media well-known by the skilled person for a given cell type.

According to one embodiment, the encapsulation buffer used in step (b) is selected from the group consisting in any biocompatible cell media.

According to a preferred embodiment, the encapsulation buffer used in step (b) is RPMI-1640 (Roswell Park Memorial Institute medium-1640) medium supplemented with GlutaMAX™, HEPES (4-(2-hydroxyethyl)-1-piperazineethanesulfonic acid) and Fetal Bovine serum (FBS).

According to another preferred embodiment, the encapsulation buffer used in step (b) is RPMI-1640 supplemented with HEPES.

According to a first variant, the encapsulation buffer used in step (b) is the same as that used in step (a).

According to a second variant, the encapsulation buffer used in step (b) is different from the one used in step (a).

According to one embodiment, the encapsulation buffer used in step (b) is selected from the group consisting in any biocompatible cell media.

According to a preferred embodiment, the encapsulation buffer used in step (a) and in step (b) is RPMI-1640 (Roswell Park Memorial Institute medium-1640) medium supplemented with GlutaMAX™, HEPES (4-(2-hydroxyethyl)-1-piperazineethanesulfonic acid) and Fetal Bovine serum (FBS).

According to another preferred embodiment, the encapsulation buffer used in step (a) is RPMI-1640 (Roswell Park Memorial Institute medium-1640) medium supplemented with GlutaMAX™, HEPES and Fetal Bovine serum and the encapsulation buffer used in step (b) is RPMI-1640 supplemented with HEPES.

According to another preferred embodiment, the encapsulation buffer used in step (a) is PBS (Phosphate Buffered Saline) and the encapsulation buffer used in step (b) is PBS (Phosphate Buffered Saline)

According to another preferred embodiment, the encapsulation buffer used in step (a) is mTeSR™ Plus supplemented with mTeSR™ Plus Supplement and the encapsulation buffer used in step (b) is mTeSR™ Plus supplemented with mTeSR™ Plus Supplement ans 20 μM of Y-27632 (Dihydrochloride).

According to another preferred embodiment, the encapsulation buffer used in step (a) is eTeSR™ supplemented with eTeSR™ Supplementand the encapsulation buffer used in step (b) is eTeSR™ supplemented with eTeSR™ Supplement.

For example, the encapsulation buffer of step (a) and step (b) can be a cell culture medium, in particular a cell culture medium adjusted to a pH ranging from 5 to 8.5, such as a pH equal to 7.6, for instance a cell culture medium adjusted to a pH of 7.6 and supplemented with 10% Fetal Bovine Serum (FBS) and 4-(2-hydroxyethyl)-1-piperazineethanesulfonic acid (HEPES).

The step (b) can be performed at a temperature comprised between 0° C. and 99° C., in particular at room temperature (25° C.).

### Step (c)

As mentioned above, the process in accordance with the present disclosure comprises the step (c).

In step (c), water-in-oil droplets are formed by encapsulating in co-flow through the two inlet channels the first and second solutions of steps (a) and (b) and an oily phase.

The main advantage of the microfluidic technology allows the good control over the droplet-formation process. Microfluidics have the advantage of providing channels of reproducible, precise dimensions as well as more complex geometries.

The inlet channels used in the present disclosure are well known by the skilled in the art. The skilled person clearly knows in particular the length, the shape (straight, curved, T, cross, Y, etc.) to be used depending on the wished application. For example, the inlet channel can be of any type compatible with channels and chambers to handle and manipulate fluids at low Reynolds numbers (lower than 2000).

According to another particular embodiment, the oily phase comprises a carrier oil with a surfactant.

According to another particular embodiment, the oily phase comprises a carrier oil without a surfactant.

The carrier oil can be selected from the group consisting of a fluorinated oil, a silicone oil, a rapeseed oil, a mineral oil, a droplet oil, or any combination thereof.

Examples of fluorinated oil (or fluorous oil) include, but are not limited to; perfluorocarbons such as perfluorooctane, perfluoroheptane, perfluorohexane (FC-72), perfluoro-1,3-dimethyl-cyclohexane, octadecafluorodecahydronaphthalene (perfluorodecalin): perfluorinated oils such as perfluoro 2-butyltetrahydrofuran, perfluoro-N-methylmorpholine (FC-3284), perfluorotripentylamine (FC-70), perfluorotributylamine (FC-43), perfluorotripropylamine (FC-3283), a perfluorotributylamine and perfluoro (dibutylmethylamine) mixture (FC-40); and hydrofluoroethers such as 2-(trifluoromethyl)-3-ethoxydodecafluorohexane (HFE-7500), ethyl perfluorobutyl ether (HFE-7200), 3-methoxyperfluoro (2-methylpentane) (HFE7300), a methyl perfluoroisobutyl ether/methyl perfluorobutyl ether mixture, a mixture of methoxynonafluorobutane and methoxynonafluoroisobutane (HFE7100), methoxy-nonafluorobutane and methoxyheptafluoropropane (HFE7000).

For example, the oily phase comprises a fluorinated oil such as perfluorohexane, perfluorooctane, and 2-(trifluoromethyl)-3-ethoxydodecafluorohexane (HFE-7500), with or without a fluorosurfactant, for instance 2-(trifluoromethyl)-3-ethoxydodecafluorohexane with a fluorosurfactant.

The step (c) can be performed at a temperature comprised between 0° C. and 99° C., in particular at room temperature (25° C.).

### Step (d)

As mentioned above, the process in accordance with the present disclosure comprises the step (d).

During the step (d), the water-in-oil droplets are polymerized at a temperature comprised between 0.01 and 99° C., in particular between 1 and 50° C., for instance at 4° C., room temperature or 37° C. to obtain the modified dextran hydrogel microparticles as defined in the present disclosure in oil. Needless to say, that the skilled person would adapt the temperature of this step depending on the desired application.

The polymerization speed of water-in-oil droplets in accordance with the present disclosure can be changed by changing the polymerization temperature or pH of encapsulation buffer.

For example, step (d) is implemented in a curve or curled channel at 37° C. for centering the cell(s) in the droplets.

Step (d′)

As mentioned above, the process in accordance with the present disclosure may comprise a step (d′).

During the step (d′), a capillary tube is implemented at a chip outlet. Said tube has to be long enough to allow a polymerization, in particular a total or a partial polymerization of the water-in-oil droplets and said tube is in contact with a heating element (solid, liquid or gaseous), in particular said tube is wrapped around a heating element or in particular said tube is immerged in a water bath, at a temperature comprised between 0.01 and 99° C., in particular between 1 and 50° C., for instance at 4° C., room temperature or 37° C. The produced droplets are collected through the long tube.

According to a particular embodiment, said capillary tube has an internal surface which is hydrophobic, for example made of metal, glass or polymer material, for instance PTFE (polytetrafluoroethylene).

According to a particular embodiment, the length of said capillary tube is comprised for example between 1 m and 10 m, in particular comprised between 3 m and 7 m, for example a length of 5 m.

The tube length is calculated to ensure that the droplet remained in the flow for a duration comprised for example between 5 min to 30 min, in particular comprised between 7 min to 15 min, for instance approximately 10 minutes, which is sufficient for the polymerization, in particular a total or partial polymerization (depending on the desired porosity for a given application) of the modified dextran hydrogel microparticles as defined in the present disclosure, at a temperature comprised between 0.01 and 99° C., in particular between 1 and 50° C., for instance at 4° C., room temperature or 37° C.

According to a particular embodiment, said capillary tube has an inner diameter comprised for example between 0.1 mm and 10 mm, in particular an inner diameter comprised between 0.2 mm and 0.5 mm, for example an inner diameter of 0.3 mm.

More particularly, the following mathematical formula allows to determine the length and the diameter of said tube, as well as the rate inside said tube:

$$\begin{matrix}\frac{{t}_{p a s s i n g}}{{t}_{p o l}} > 1 \\ {t}_{p a s s i n g} = \frac{π {d}^{2}}{4} \cdot \frac{L}{Q}\end{matrix}$$

wherein t_{passing} is the time that needs a droplet to flow from the entry to the exit of the tube, tₚₒₗ is the time at given temperature T, for the given gel to pass the transition G′ >G″ (that is to say G′ is higher than G″, G′ being a storage modulus, and G″ being a loss modulus); d is a tube diameter; L is the length of the tube, and Q is a flowrate in the tube. So, the parameters Q, d and L, as well as T, should be chosen to keep the proportion between t_{passing} and tₚₒₗ in the range indicated in the equation.

According to a particular embodiment, said heating element is solid, liquid or gaseous.

According to a particular embodiment, said tube is wrapped around a heating element.

According to a particular embodiment, said heating element is a Peltier element in particular comprises a metallic block such an aluminum block.

According to a particular embodiment, said tube is immerged in a water bath, According to a particular embodiment, said heating element is configured to heat at a temperature comprised between 0.01 and 99° C., in particular between 1 and 50° C., for instance at 4° C., room temperature or 37° C.

Said step (d′) is particularly advantageous to be implemented in the process in accordance with the present disclosure to enable the cell centering of the at least one natural or synthetic cell as defined in the present disclosure comprised in the modified dextran hydrogel microparticle as defined in the present disclosure. The advantages of such a cell centering are explained in the present disclosure and an example of cell centering is detailed in example 6 below.

Indeed, thanks to the implementation of such a long capillary tube, the at least one natural or synthetic cell as defined in the present disclosure, in particular a spheroid or an organoid, is more centered within the droplets during the polymerization of the droplets, enabling the cells to grow larger without compromising the gel's integrity (as shown in example 6 below). The fact that the at least one natural or synthetic cell is more centered within the modified dextran hydrogel microparticles as defined in the present disclosure also allows to increase the viability of the cells, probably explained by a higher protection of the hydrogel layer when the cells are centered, compared when cells are in the border of the hydrogel bead.

As illustrated below in example 6, the centering of the at least one natural or synthetic cells within droplets also offers a simpler solution compared to the re-encapsulation of hydrogel beads into droplets.

Further, advantageously, this method of centering does not require the design of a specialized microfluidic chip and is a flow-based method, enabling the production of polymerized particles with centered cells immediately after droplet generation, and ensuring gentle polymerization conditions.

Furthermore, this method enables better clustering of membrane protein such as CD3 receptors (as shown in example paragraph 6.1.3 below) and it opens the possibility to perform functional cell assays with high throughput and at the single cell level.

### Step (e)

As mentioned above, the process in accordance with the present disclosure may comprise the step (e). The step (e) can be implemented either after the step (d) or after the step (d′) as defined above.

After obtaining the modified dextran hydrogel microparticles in oil, an optional supplemental step can consist in extracting the modified dextran hydrogel microparticles from the oily phase by washing, extracting, demulsification, electric fields destabilization, chemical destabilization and/or oil evaporation, for obtaining modified dextran hydrogel microparticles in an aqueous solution.

The washing, extracting, demulsification, electric fields destabilization, chemical destabilization and/or oil evaporation are well-known steps or methods for the skilled person.

For example, the washing of the beads or microparticles can be carried out with an appropriate volume of emulsion fresh fluorinated oil such as HFE-7500 oil without surfactant. Then, the beads can be centrifuged, and the rest of oil can be removed. Then, an emulsion volume of corresponding cell medium or another aqueous phase can be added.

Then, emulsion volume of ¹H, ¹H,2H,2H-perfluoro-1-octanol can be added, and then it was waited till the beads go up to aqueous phase. 1H, 1H,2H,2H-perfluoro-1-octanol can then be removed from the bottom of the tube, using pipette or syringe with attached needle. The tube can then be centrifuged for instance at 100 g for 20s, and the rest of the 1H, ¹H,2H,2H-perfluoro-1-octanol can be removed.

According to a particular embodiment, the w/v percentage of the at least one vinyl sulfone functionalized dextran as defined in the present disclosure and the at least one crosslinkable polymer as defined in the present disclosure is comprised between 1% and 30%, in particular between 2% and 15%, and more particularly between 2.5% and 12%, for example between 2.8% and 10% with respect to the volume of the first and second solutions of step (a) and (b) as defined in the present disclosure.

According to a preferred embodiment, the w/v percentage of the at least one vinyl sulfone functionalized dextran as defined in the present disclosure and the PEG dithiol as defined in the present disclosure is comprised between 1% and 30%, in particular between 2% and 15%, and more particularly between 2.5% and 12%, for example between 2.8% and 10% with respect to the volume of the first and second solutions of step (a) and (b) as defined in the present disclosure.

### Step (f)

According to one embodiment, a passage step may be implemented after steps (a) to (e), meaning that the cell encapsulated microparticles as obtained in the herein above described steps may be transferred or moved to another environment or condition according to experimental procedures known to the man skilled in the art.

In one embodiment, the hydrogel microparticles of beads according to the disclosure can be frozen for cell preservation or other cryogenic applications as detailed herein after.

Further provided herein is a process for encapsulating at least one synthetic or natural cell comprising at least:
(a) Providing a first solution which comprises the at least one synthetic or natural cell, in particular a cell, in an encapsulation buffer and at least one vinyl sulfone functionalized dextran:
(b) A step of using a microfluidic device, in particular comprising at least two inlet channels that converge into a droplet generation region for encapsulating said synthetic or natural cell into modified dextran hydrogel microparticles as defined above, in particular comprising (i) at least one vinyl sulfone functionalized dextran as defined above and (ii) at least one crosslinkable polymer having at least two thiol functions as defined above.

Further provided herein is a process for encapsulating at least one synthetic or natural cell comprising at least:
(a) Providing a first solution which comprises the at least one synthetic or natural cell, in particular a cell, in an encapsulation buffer and at least one vinyl sulfone functionalized dextran:
(b) Providing a second solution which comprises an encapsulation buffer, identical or different from the buffer used in step (a), and the at least one crosslinkable polymer having at least two thiol functions as defined above:
(c) Forming water-in-oil droplets by encapsulating in co-flow through the two inlet channels the first and second solutions of steps (a) and (b) and an oily phase:
(d) Polymerizing the water-in-oil droplets at a temperature comprised between 0.01 and 99° C., in particular between 1 and 50° C., for instance at 4° C., room temperature or 37° C. to obtain the modified dextran hydrogel microparticles in oil; and
(d′) Optionally implementing during said step (d) a capillary tube at a chip outlet, said tube being long enough to allow a polymerization, in particular a total or a partial polymerization of the water-in-oil droplets, and said tube being in contact with a heating element (solid, liquid or gaseous), in particular said tube being wrapped around a heating element or in particular said tube being immerged in a water bath, at a temperature comprised between 0.01 and 99° C., in particular between 1 and 50° C., for instance at 4° C., room temperature or 37° C.; and
(e) Optionally extracting the modified dextran hydrogel microparticles from the oily phase by washing, extracting, demulsification, electric fields destabilization, chemical destabilization and/or oil evaporation, for obtaining modified dextran hydrogel microparticles in an aqueous solution.

### In Vitro Methods

Herein is also provided an in vitro method for cultivating at least one cell comprised in the modified dextran hydrogel microparticle as defined in the present disclosure, comprising at least a step of incubating the microparticle in an environment, suitable to allow for cell survival, cell growth, cell differentiation and/or cell proliferation such as clonal expansion, more particularly for producing a cell cluster, a spheroid, an organoid, a gastruloid, a tumoroid or a tissue.

An environment suitable to allow for cell survival, cell growth, cell differentiation and/or cell proliferation in accordance with the present disclosure is any environment well known by the skilled person. For example, such an environment can be cell culture medium, cell differentiation medium, any cell activator medium, allowing cell engagement or cell sedation, or any cell compatible medium.

The step of incubating the microparticle(s) can be a static incubating step or a dynamic incubating step, in particular a dynamic incubating step.

Advantageously, the dynamic incubating step can enable to optimize the culture of the at least one natural or synthetic cell by increasing the growth rate of the at least one natural or synthetic cell and/or by increasing the cell culture yield for a given cell medium volume.

According to another advantage, the dynamic incubating step can enable to optimize the production of said compounds, proteins, polypeptides, oligopeptides, nucleic acids, cell secretome or antibodies from said at least one synthetic or natural cell by increasing the production yield, that is to say by increasing the amount of said compounds, proteins, polypeptides, oligopeptides, nucleic acids, cell secretome or antibodies secreted by said at least one synthetic or natural cell.

According to another advantage, the microparticle(s) in accordance with the present disclosure enable(s) the culture of said at least one synthetic or natural cell and/or the production of said compounds, proteins, polypeptides, oligopeptides, nucleic acids, cell secretome or antibodies from said at least one synthetic or natural cell via a dynamic incubating step and this whatever the at least one synthetic or natural cell is adherent or not-adherent. Usually, the adherent cells are only cultured in static (adherent cell culture involves cultivating cells as monolayers on an artificial substrate). Hence, the encapsulation of one or more adherent cell(s), such as H1975 cells in the microparticle(s) in accordance with the present disclosure allows to culture the cell(s) in dynamic.

Herein is also provided an in vitro method for screening compounds, proteins, polypeptides, oligopeptides, nucleic acids, cell secretome or antibodies comprising using a modified dextran hydrogel microparticle as defined in the present disclosure comprising cultivated cells according to the in vitro method according to the present disclosure. According to a particular embodiment, the present disclosure relates to an in vitro method for screening compounds comprising using a modified dextran hydrogel microparticle as defined in the present disclosure comprising cultivated cells according to the in vitro method according to the present disclosure. For example, the compounds can be therapeutic compounds, like small molecules.

According to another particular embodiment, the present disclosure relates to an in vitro method for screening proteins, polypeptides, oligopeptides, nucleic acids comprising using a modified dextran hydrogel microparticle as defined in the present disclosure comprising cultivated cells according to the in vitro method according to the present disclosure. For example, the proteins, polypeptides, oligopeptides, nucleic acids can be protein A/G, streptavidin, RGD-Cys, plasmids, linear DNA fragment, immunoglobulins.

According to another particular embodiment, the present disclosure relates to an in vitro method for screening cell secretome comprising using a modified dextran hydrogel microparticle as defined in the present disclosure comprising cultivated cells according to the in vitro method according to the present disclosure.

According to another particular embodiment, the present disclosure relates to an in vitro method for screening antibodies comprising using a modified dextran hydrogel microparticle as defined in the present disclosure comprising cultivated cells according to the in vitro method according to the present disclosure.

As a matter of example, the antibody can be an immunoglobulin such as IgG (Immunoglobulin G) (see example 2 below), an IgG fragment such as Fab, F(ab)₂ or Fc, and VHH.

The antibodies can be captured or caught by any means or methods well known by the skilled person. For example, magnetic beads which are superparamagnetic, non-aggregating iron oxide particles (or “microspheres”) can be used for capturing antibodies. In the experimental part, and more particularly in Example 2 herein after is illustrated the screening of IgG secreting cells by using the encapsulation of Chinese hamster ovary cells in modified dextran hydrogel microparticles as defined in the present disclosure. As described in this example, magnetic beads were prepared for catching (or capturing) human IgG. Magnetic beads can be obtained by any method well known by the skilled in the art. For example, as illustrated in example 2 herein after, a method can comprise the following steps:
1. Taking an appropriate volume such as 60 μL of bead Streptavidin for 100 μL of final solution.
2. Putting off the supernatant using magnet.
3. Adding an appropriate volume such as 6 μL of VHH anti-human Kappa.
4. Incubating an appropriate time such as at least for 45 min at a temperature comprised between 20° C. and 25° C., such as room temperature
5. Putting off the supernatant using magnet.
6. Washing beads with an appropriate volume of a buffer such as 90 μL DPBS (Dulbecco Phosphate Buffered Saline) twice.
7. Resuspending in an appropriate volume such as 100 μL final buffer.
8. Just before every encapsulation: Vortex beads, sonicating the beads: 1 min on “sweep” 37 kHz 80% Power and 1 min on pulse 80 KHz 80% P in ice and vortex again.

As mentioned above, the process according to the present disclosure allows to obtain different mechanical properties of the hydrogel microbeads so that it enables to adapt the hydrogel following the cell origin and makes possible the survival and/or the growing of the cells of interest. For example, a cell from bones needs a hard material, at the opposite a cell from blood needs a soft material.

In addition, the high elasticity of the material of the beads made it possible to grow tumoroids larger than the beads itself without rupture.

In one embodiment, the in vitro method for cultivating at least one cell comprised in the modified dextran hydrogel microparticle as defined in the present disclosure produces spheroids or tumoroids.

Spheroids may be used to model various diseases, including cancer, and to study tissue development and regeneration. They may provide a platform for understanding disease mechanisms, testing therapeutic strategies, and exploring regenerative medicine applications. They may mor particularly be used for drug screening and cancer research. In one embodiment, the in vitro method for cultivating at least one cell comprised in the modified dextran hydrogel microparticle as defined in the present disclosure may produce hundreds of thousands of tumoroids in a single experiment.

Tumoroids are typically valuable for preclinical drug testing and screening. Their 3D structure and microenvironment allow for a realistic assessment of drug penetration, efficacy, and resistance. Tumoroids may be used to study various aspects of cancer, including tumor growth, metastasis, angiogenesis, and the tumor microenvironment.

They may also be employed to investigate the effects of different therapies, including chemotherapy, targeted therapy, and immunotherapy.

The hydrogel microparticles according to the present disclosure are of particular interest in this field as it allows, as mentioned above, to customize them and to reach versatility to serve the research needs. Namely, hydrogel beads can be customized to include specific biochemical and mechanical properties that influence tumor cell behavior. This versatility allows researchers to create tumoroid models that closely mimic different types of tumors and their specific microenvironments.

As a matter of example, tumoroids (or spheroids) of MKN45 cell line, of HEP3B cell line, of IGROV1 cell line, of MDA-MB-468 cell line, of CHO cell line, of H1975 cell line, and the like may be produced as in example 1 or example 2 herein after.

Advantageously, the spheroids produced according to the present disclosure are also compatible with RNA extraction for subsequent RNA sequencing (RNASEQ) analysis. As illustrated in example 1.9 below, for example, the spheroids produced from MKN45 and IGROV1 cells can be treated with dextranase and NGS (Next-Generation Sequencing), a technology used for RNA sequencing, can then be performed. It has been demonstrated that there is an impact of three-dimensional culture on cellular transcriptional activity. The compatibility of these spheroids with RNA extraction and RNA sequencing also facilitates their use in advanced genomic studies, paving the way for more detailed and accurate molecular characterizations in cancer research.

According to a particular embodiment, the present disclosure relates to an in vitro method for spheroid, organoid preparation for diagnostic purposes including precision medicine, comprising using a modified dextran hydrogel microparticle as defined in the present disclosure comprising cultivated cells according to the in vitro method according to the present disclosure.

According to a particular embodiment, the present disclosure relates to an in vitro method for spheroid, organoid preparation for in vitro clinical trials, comprising using a modified dextran hydrogel microparticle as defined in the present disclosure comprising cultivated cells according to the in vitro method according to the present disclosure.

According to a particular embodiment, the present disclosure relates to an in vitro method for spheroid, organoid preparation for in vitro preclinical experiments including toxicology experiments and drug metabolism and pharmacokinetics, comprising using a modified dextran hydrogel microparticle as defined in the present disclosure comprising cultivated cells according to the in vitro method according to the present disclosure.

According to a particular embodiment, the present disclosure relates to an in vitro method for spheroid, organoid preparation for cell or tissue therapies, comprising using a modified dextran hydrogel microparticle as defined in the present disclosure comprising cultivated cells according to the in vitro method according to the present disclosure.

Further, advantageously, the spheroids produced according to the present disclosure can be stained and it has been shown by the inventors that they are compatible with immunofluorescence (IF) methods (see example 1.10 below).

The staining can be performed according to any method well known in the art by the skilled person, for example, according to the protocol described in Dekkers et al., Nature protocols, 2019, 14 (6), 1756-1771 as explained in example 1.10 below. As indicated in example 1.10 below; this protocol can be changed in that fixation of spheroids by PFA (paraformaldehyde) can be performed before gel dissolution, and dissolution of the gel can be made by 1/2000 solution of dextranase in 0.1% (v/v) PBS-Tween buffer right after the fixation.

For instance, antibodies such as primary antibodies and secondary antibodies can be used, as well as dyes (staining).

By way of examples, among the primary antibodies can be cited anti-Ki67 such as anti-Ki67 from Rabbit or anti-E-cadherin such as anti-E-cadherin from Mouse.

By way of examples, among the secondary antibodies can be mentioned Donkey anti-Rabbit IgG Alexa Fluor® 488 or Donkey anti-Mouse IgG Alexa Fluor® 594.

By way of examples, among the staining can be cited Phalloidin Alexa Fluor® plus 647, or DAPI (4′,6-diamidino-2-phenylindole).

In general, for the staining, any chemical or biological molecules, as well as particles could be used, that could penetrate in the gel, so all of the molecules and particles less than 700 kDa.

Herein is also provided an in vitro method for producing compounds, proteins, polypeptides, oligopeptides, nucleic acids, cell secretome or antibodies from at least one synthetic or natural cell encapsulated in the modified dextran hydrogel microparticle(s) as defined in the present disclosure, comprising at least:
a step of cultivating and/or of operational maintaining of the at least one synthetic or natural cell: or
a step of production of said compounds, proteins, polypeptides, oligopeptides, nucleic acids, cell secretome or antibodies from said at least one synthetic or natural cell: or
a step of cultivating and/or of operational maintaining of the at least one synthetic or natural cell, and a step of production of said compounds, proteins, polypeptides, oligopeptides, nucleic acids, cell secretome or antibodies from said at least one synthetic or natural cell.

The step of cultivating and/or of operational maintaining of the at least one synthetic or natural cell may comprise a sub-step of incubating the microparticle(s) in accordance with the present disclosure in an environment, such as a cell medium, suitable to allow cell survival, cell growth, cell differentiation, cell proliferation, such as clonal expansion, more particularly for producing a cell cluster, a spheroid, an organoid, a gastruloid, a tumoroid or a tissue, and/or operational maintenance that is to say to maintain the functions of the at least one cell, in particular synthetic cell.

This sub-step of incubating the microparticle(s) can be a static incubating step or a dynamic incubating step, in particular a dynamic incubating step.

The production step may comprise at least one sub-step of incubating the microparticle(s) in accordance with the present disclosure in an environment, such as a cell medium, suitable to allow production of compounds, proteins, polypeptides, oligopeptides, nucleic acids, cell secretome or antibodies from said at least one synthetic or natural cell.

This step of incubating the microparticle(s) can be a static incubating step or a dynamic incubating step, in particular a dynamic incubating step.

The production step can also comprise at least one sub-step of adding a production inducer, such as a saccharide (a monosaccharide or a polysaccharide), in said environment, notably in said cell medium.

According to a first variant, an in vitro method for producing compounds, proteins, polypeptides, oligopeptides, nucleic acids, cell secretome or antibodies from at least one synthetic or natural cell encapsulated in the modified dextran hydrogel microparticles as defined in the present disclosure, comprises at least a step of cultivating and/or of operational maintaining of the at least one synthetic or natural cell.

According to a second variant, an in vitro method for producing compounds, proteins, polypeptides, oligopeptides, nucleic acids, cell secretome or antibodies from at least one synthetic or natural cell encapsulated in the modified dextran hydrogel microparticles as defined in the present disclosure, comprises at least a step of production of said compounds, proteins, polypeptides, oligopeptides, nucleic acids, cell secretome or antibodies from said at least one synthetic or natural cell.

According to a third variant, an in vitro method for producing compounds, proteins, polypeptides, oligopeptides, nucleic acids, cell secretome or antibodies from at least one synthetic or natural cell encapsulated in the modified dextran hydrogel microparticles as defined in the present disclosure, comprises at least a step of cultivating and/or of operational maintaining of the at least one synthetic or natural cell, and a step of production of said compounds, proteins, polypeptides, oligopeptides, nucleic acids, cell secretome or antibodies from said at least one synthetic or natural cell.

For example, the cells, in particular CHO cells as shown in example 2 below, embedded in the hydrogel beads in accordance with the present disclosure can be easily manipulated. In addition, they have better oxygenation in a suitable medium culture, and better access to nutriments. Consequently, they have a higher compound, protein, polypeptide, oligopeptide, nucleic acid, cell secretome or antibody production yield, particularly a higher antibody production yield, in particular a higher IgG production yield. Herein is also provided an in vitro method for testing a drug compound comprising using at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof, produced in a modified dextran hydrogel microparticles as defined in the present disclosure.

According to one embodiment, the present disclosure relates to an in vitro method for testing a drug compound comprising at least:
a step of culturing at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof, produced according to the in vitro method of the present disclosure,
a step of exposing said at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof, to the drug compound,
a step of measuring a biological response of interest,
a step of comparison of the biological response with a predetermined value or with a control condition wherein said at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof, has not been exposed to said drug compound, thereby assessing the biological response of the tested drug compound.

The biological response of interest may be any relevant biological response allowing to provide information on the effect or non-effect of the addition of said drug compound, for example the biological response of interest may be selected from cell morphology: cell viability: activation of a cell: physiology of a cell: secretion of a cell: potency of a cell: presence, quantity and sequence of RNA molecules: presence and concentration of compounds: etc.

These kinds of biological responses of interest may be measured by any methods known in the art and for example as explained in the below Method for Quality control part. Needless to say, the biological response of interest may depend on the nature or on the type of the cell and its specific physiology.

In one embodiment, said in vitro method is for testing a drug compound for activity against a disease comprising at least:
a step of culturing at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof, produced according to the in vitro method of the present disclosure,
a step of exposing said at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof, to the drug compound,
a step of measuring a biological response of interest with the disease,
a step of comparison of said biological response of interest with the disease with a predetermined value or with a control condition wherein said at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof has not been exposed to said drug compound, thereby assessing the activity against a disease of the tested drug compound.

In one embodiment, said in vitro method is for testing a drug toxicity comprising at least:
a step of culturing at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof, produced according to the in vitro method of the present disclosure,
a step of exposing said at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof to the drug compound,
a step of measuring a biological response revealing toxicity,
a step of comparison of said biological response revealing toxicity with a predetermined value or with a control condition wherein said at least one cell, cell cluster, spheroid, organoid, gastruloid, tumoroid or tissue as defined in the present disclosure, in particular said at least one cell, cell cluster, spheroid, organoid, gastruloid, tumoroid or tissue comprising at least one synthetic or natural cell as defined in the present disclosure, in particular said at least one synthetic or natural cell being selected from connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THP1B cell, cell-free systems such as cell extract-based cell free systems and purified enzyme-based cell free systems and combinations thereof, has not been exposed to said drug compound, thereby assessing the toxicity the tested drug compound.

### KIT

Herein is also provided a kit for making the microparticle encapsulating at least one synthetic or natural cell according to the present disclosure, the kit comprising: at least one vinyl sulfone functionalized dextran as defined in the present disclosure, a culture medium as defined in the present disclosure: at least one crosslinkable polymer having at least two thiol functions as defined in the present disclosure: an oily phase as defined in the present disclosure: a microfluidic chip as defined in the present disclosure; and optionally instructions for use.

The culture medium in accordance with the present disclosure can be any appropriate culture medium well known by the skilled person. For example, among the culture media can be cited those already cited above, that is to say any biocompatible cell media, adjusted or not to a particular pH, containing or not containing fetal serum (bovine, calf, horse, pig, sheep, or goat), biocompatible buffer solutions, for example PBS and DPBS (Dulbecco Phosphate Buffered Saline) with or without supplementary salts, protein mix or surfactant, as well as any cell-free buffers and solutions, and mixtures thereof.

Microfluidic chips come in all shapes and sizes, as long as those sizes are “small.” The microfluidic chip in accordance with the present disclosure can be any appropriate microfluidic chip well known by the skilled person. For example, among the microfluidic chip can be cited a microfluidic chip which consists of microchannels, and chambers made of glass, silicon, or polymer materials.

According to a particular embodiment, the present disclosure relates to a kit comprising: at least one vinyl sulfone functionalized dextran as defined in the present disclosure, a culture medium as defined in the present disclosure; at least PEG dithiol as defined in the present disclosure: an oily phase as defined in the present disclosure; a microfluidic chip as defined in the present disclosure; and optionally instructions for use.

According to another particular embodiment, the disclosure relates to a kit comprising at least one vinyl sulfone functionalized dextran as defined in the present disclosure, a linker and a microfluidic chip, for the high throughput testing of drugs using robotized methods.

### Microfluidic or Millifluidic Circuit or Channel

Herein is also provided a microfluidic or millifluidic circuit or channel comprising the modified dextran hydrogel microparticles as defined in the present disclosure.

In the sense of the present disclosure:
A microfluidic channel means that the channel cross-section has a greatest dimension higher than or equal to 0.5 μm and lower than 1000 μm, in particular higher than or equal to 1 μm and lower than 1000 μm:
A millifluidic channel means that the channel cross-section has a greatest dimension higher than or equal to 1 mm and lower than 100 mm, in particular higher than or equal to 1 mm and lower than 10 mm.

According to one embodiment, said modified dextran hydrogel microparticles as defined in the present disclosure are flowing and/or are stored inside said microfluidic or millifluidic circuit or channel.

Generally, when a non-encapsulated cell is injected in a microfluidic or millifluidic circuit or channel, mechanical constraints, such as shear stress and mechanical shocks, can apply on said cell.

Thanks to the encapsulation of the at least one natural or synthetic cell in a modified dextran hydrogel microparticles in accordance with the present disclosure, the at least one natural or synthetic cells is protected from said mechanical constraints. Indeed, the modified dextran hydrogel microparticles in accordance with the present disclosure has a role of mechanical protection of the at least one natural or synthetic cell.

Moreover, the encapsulation of the at least one natural or synthetic cell in a modified dextran hydrogel microparticles in accordance with the present disclosure allows to eliminate the adherence of the cells to the wall(s) of the microfluidic or millifluidic circuit or channel, which thus enables better flowing of the cells inside said circuit or channel.

Furthermore, the encapsulation of the at least one natural or synthetic cell in a modified dextran hydrogel microparticles in accordance with the present disclosure, allows to eliminate the Poisson distribution observed with non-encapsulated cells which can occur both during storage and when flowing through the channels.

For example, a tumoroid encapsulated inside a hydrogel bead in accordance with the present disclosure may be injected inside a microfluidic channel. Thanks to the hydrogel shell, the hydrogel beads in accordance with the present disclosure containing the tumoroid will slide and move easily inside the tiny channel. In particular, the hydrogel shell will prevent the inside tumoroid from the shear stress and mechanical stress. Indeed, without the hydrogel shell, a naked tumoroid will have a high risk to stuck to the channel walls and/or to be destroyed by the fluidic or mechanical stresses.

Process for Encapsulating a Modified Dextran Hydrogel Microparticles in Accordance with the Present Disclosure

Herein is also provided a process for encapsulating in a compartment, in particular in an aqueous or in a hydrogel droplet, modified dextran hydrogel microparticle(s) in accordance with the present disclosure comprising at least one step of injection of said modified dextran hydrogel microparticle(s) in accordance with the present disclosure in a microfluidic or millifluidic circuit or channel.

According to one embodiment, said microfluidic or millifluidic circuit or channel is at least partially filled with an oil phase or an aqueous phase or a hydrogel phase.

### Microparticles Encapsulated in a Compartment

Herein is also provided a modified dextran hydrogel microparticle in accordance with the present disclosure encapsulated in a compartment, in particular in an aqueous or a hydrogel droplet.

Said encapsulated modified dextran hydrogel microparticle may be obtained thanks to the previously defined process for encapsulating modified dextran hydrogel microparticle(s) in accordance with the present disclosure.

The compartment, in particular the aqueous or the hydrogel droplet, may comprise at least one element selected from:
any cell as defined in the present disclosure, for instance an immune cell, such as a lymphocyte T cell, in particular a Jurkat cell; and/or a cytotoxic T cell:
a drug compound, in particular suitable for activating said cell, for instance an immune cell, such as a T cell engager, and/or a NK engager:
a compound allowing to dissolve the modified dextran hydrogel of the microparticle according to the present disclosure while maintaining the biological functions of the at least one natural or synthetic cell encapsulated in said microparticle, such as dextranase; and
mixtures thereof.

Said compound allowing to dissolve the modified dextran hydrogel of the microparticle according to the present disclosure while maintaining the biological functions of the at least one natural or synthetic cell encapsulated in said microparticle may depend on the used linker in the modified dextran hydrogel microparticles in accordance with the present disclosure.

Said compound allowing to dissolve the modified dextran hydrogel of the microparticle according to the present disclosure while maintaining the biological functions of the at least one natural or synthetic cell encapsulated in said microparticle, may enable the interaction between the at least one natural or synthetic cell encapsulated in the modified dextran hydrogel microparticle as defined in the present disclosure and the cell comprised in the compartment as defined in the present disclosure, for instance an immune cell, as well the interaction between the at least one natural or synthetic cell encapsulated in the modified dextran hydrogel microparticle as defined in the present disclosure and said drug compound.

Indeed, the porosity of the modified dextran hydrogel microparticle according to the present disclosure may allow the passage between the microparticle according to the present disclosure and the compartment of the drug compound which may be comprised in the compartment and/or of the compounds, proteins, polypeptides, oligopeptides, nucleic acids, cell secretome or antibodies produced by the at least one natural or synthetic cell encapsulated in the microparticle according to the present disclosure. However, the porosity does not allow the passage between the microparticle according to the present disclosure and the compartment of natural cells and of some synthetic cells. That is the reason why the compound allowing to dissolve the modified dextran hydrogel of the microparticle according to the present disclosure while maintaining the biological functions of the at least one natural or synthetic cell encapsulated in said microparticle, such as dextranase, is needed, for example, in order to improve the interaction between the at least one natural or synthetic cell encapsulated in the modified dextran hydrogel microparticle as defined in the present disclosure and the cell comprised in the compartment as defined in the present disclosure, for instance an immune cell, as well the interaction between the at least one natural or synthetic cell encapsulated in the modified dextran hydrogel microparticle as defined in the present disclosure and said drug compound.

### Method for Quality Control

Herein is also provided a method for the quality control of a batch of modified dextran hydrogel microparticles as defined in the present disclosure, comprising at least:
a step of recovering of a sample of microparticles from said batch:
a step of measuring at least one parameter for each microparticle of said sample:
a step of comparison of the value of the at least one measured parameter for each microparticle of said sample with a predetermined value:
a step of determination based on said comparison whether or not each microparticle of said sample has the required quality; and
a step of extrapolation of the results of the previous determination step to said batch.

According to one embodiment, said sample comprises between 1 and 100 000 microparticles, in particular between 1 and 1 000 microparticles.

According to one embodiment, the at least one parameter for each microparticle of said sample is related to the at least one natural or synthetic cell encapsulated within said microparticle.

According to one embodiment, the at least one parameter is selected from the viability of the at least one natural or synthetic cell, the morphology of the at least one natural or synthetic cell, the potency of the at least one natural or synthetic cell, the presence, the quantity and the sequence of RNA molecules in the at least one natural or synthetic cell, the presence and/or the concentration of at least one compound, protein, polypeptide oligopeptides, nucleic acid, cell secretome or antibody secreted by the at least one natural or synthetic cell.

The at least one parameter as defined above can be measured by any method well known by the skilled person.

For example, the viability of the at least one natural or synthetic cell can be measured/assessed by commercial kit for the viability assessment, or by phase contrast microscopy, or by fluorescent microscopy, or by using specific coloration of cells (like Trypan Blue), or by assessment of ATP content (for example line of products CellTyter-Glo (Promega) or RealTime-Glo (Promega)), or by FACS: or any other method known for cell viability assessment by skilled person.

For example, the morphology of the at least one natural or synthetic cell can be measured/assessed by brightfield or fluorescent imaging, as well as by fluorescent profilometer, or by combination of artificial intelligence algorithms and imaging, or any other method known for cell viability assessment by skilled person.

For example, the potency of the at least one natural or synthetic cell can be measured by a functional assay, an analysis of the cell secretome.

For example, the presence, the quantity and the sequence of RNA molecules in the at least one natural or synthetic cell can be measured by UV absorbance by spectroscopy, by reverse transcription PCR, by RNA-sequencing, or any other method known for cell viability assessment by skilled person.

For example, the presence and/or the concentration of at least one compound, protein, polypeptide oligopeptides, nucleic acid, cell secretome or antibody secreted by the at least one natural or synthetic cell can be measured by ELISA, or by Western Blot, or by fluorescent and brightfield imaging using specific and non-specific coloration technics, by SPR (Surface Plasmon Resonance), or NMR (Nuclear Magnetic Resonance), or by chromatography: or by sequencing, or by qPCR (quantitative Polymerase Chain Reaction), or by ddPCR (digital droplet PCR), or by reverse transcription PCR, or by mass spectrometry, or by cytometric bead array (like Luminex), or by BLI (Bio-Layer Interferometry), or by FRET (Förster Resonance Energy Transfer), or by capillary electrophoresis, of by gel electrophoresis, or by Raman Spectroscopy, or by LFA (Lateral Flow Assay), or any other method known for cell viability assessment by skilled person.

Typically, the quality control method in accordance with the present disclosure is carried out before filling wells of a culture plate such as a culture plate 96 wells or 384 wells.

Thanks to the massive-scale bioreactor-style cultivation and pre-selection method of the present disclosure, users can generate big amount of biological models in a single vessel, sample a small subset for quality assessment, and only thereafter distribute confirmed-high-quality models into assay plates or send models for other type of assays. This approach dramatically reduces the number of culture plates and reagents consumed, lowers labor and material costs associated with failed batches, and ensures that downstream drug-screening or analytical tests are performed exclusively on validated biological objects.

### Cell Therapy and Therapeutic Uses

Herein is also provided a pharmaceutical composition for use in a method of treating a patient having a disorder or condition, and/or for transplantation therapy, and/or for use to restore and/or improve the function of a tissue or of an organ of a patient, and/or for use for tissue regeneration and/or tissue repair, said pharmaceutical composition comprising the cells cultivated according to the in vitro method as defined in the present disclosure.

According to one embodiment, said cells are released from the modified dextran hydrogel microparticles in accordance with the present disclosure by any suitable means.

For example, the release of said cells is carried out by using dextranase or by any means suitable to dissolve the modified dextran hydrogel and to maintain them alive. These means depend on the used linker in the modified dextran hydrogel microparticles in accordance with the present disclosure.

According to one embodiment, said cells are alive.

According to one embodiment, the disorder or condition is selected from any degenerative, ischemic, inflammatory, metabolic, genetic or traumatic pathology affecting human health, including, but not limited to, cardiovascular disorders (e.g., heart attack, ischemic heart disease, myocardial infarction), neurological disorders (e.g., Parkinson's disease, Alzheimer's disease, stroke, spinal cord injury), metabolic diseases (e.g., diabetes mellitus types 1 and 2), musculoskeletal conditions (e.g., osteoarthritis, bone fracture, muscular dystrophy), hepatic or renal failure, chronic wound healing impairments (e.g., burn wounds, chronic skin ulcers), ophthalmological diseases (e.g., age-related macular degeneration, diabetic retinopathy), hematological disorders (e.g., anemia, hemophilia), and the like.

According to one embodiment, the targeted tissue or organ of a patient is selected from any anatomical or functional unit in which restoration or regeneration is desired, including, but not limited to, cardiac tissue, pancreatic islets, neural tissue (central or peripheral), hepatic tissue, renal tissue, pulmonary tissue, musculoskeletal tissues (bone, cartilage, tendon, muscle), dermal or epidermal layers of skin, ocular tissues (cornea, retina), hematopoietic and lymphoid organs (bone marrow, spleen), and the like.

According to one embodiment, said cells come from the same patient (autograft) or from a different patient (allograft).

According to one embodiment, said patient is healthy or ill.

According to one embodiment, said cells are administered in an animal model. According to this embodiment, said patient can be ill.

According to one embodiment, the cells are genetically modified or not genetically modified before administration to the patient.

The disclosure will now be described by means of the examples that follow, which are given by way of illustration and without limitation of the disclosure.

### Examples

### Example 1: HIGH-THROUGHPUT SPHEROIDS PRODUCTION, Characterization and Uses

### 1.1 Material and Methods

### 1.1.1. Used Products

1. 1H, ¹H,2H,2H-perfluoro-1-octanol (Sigma, ref. 370533)
2. 3M™ Novec™ 7500 Engineered Fluid (2-trifluoromethyl-3-ethoxy dodecafluorohexane), called HFE7500, 3M
3. Antibiotic-antimycotic, called anti-anti in the present disclosure, (Gibco, ref. 15240062)
4. StemPro™ Accutase™, called accutase in the present disclosure (Gibco, ref. A1110501)
5. Enzyme TrypLET Select, called trypsin in the present disclosure (Gibco, ref. 12563011)
6. 37 μm Reversible Strainer, (Stemcell, 27215)
7. Protein LoBind Tube 1.5 mL (Eppendorf, 022431081)
   a. Protein LoBind Conical Tubes 15 mL (Eppendorf, 0030122216)
   b. Protein LoBind Conical Tubes 50 mL (Eppendorf, 0030122240)
8. 4-(2-hydroxyethyl)-1-piperazineethanesulphonic acid (HEPES) (1M), (Gibco, 15630-080)
9. SH-PEG-SH (PEG dithiol) 1.5 kDa, (Merck, JKA4105-1G)
10. Dextran-VS (Dextran-Vinyl sulfone) 70 kDa, DS=22% (degree of substitution)
11. SH-RGD, (Cellendes, 3-D Life RGD (Arginyl-glycyl-aspartic acid) Peptide, 09-P-001)
12. CellTiter-Glo® 2.0 Cell Viability Assay, (Promega, G9241)
13. Dextranase (Sigma, ref. D0443)
14. Fluorosurfactant (RAN Biotechnologies, Neat (un-dissolved) 008-FluoroSurfactant)
15. T25 non-adherent cell culture flask, (Greiner, 690195)
16. T25, T75 cell culture flask (Thermo Scientific 156367, Thermo Scientific 156499)
17. Poly-d-Lysine, (Gibco, A3890401)
18. Dextran-VS (Dextran-Vinyl sulfone) 70 kDa, DS=33% (degree of substitution)

### Materials for Preparing Dextran-VS 70 kDa:

Dextran T70: Pharmacosmos
Divinyl Sulfone (DVS): 97% Thermo Scientific CAS: 77-77-0
NaOH: Sigma Aldrich CAS: 1310-73-2
HCl: Sigma Aldrich CAS: 7647-01-0
Dialysis membrane: Spectra/Por 3.5 kD (Spectrum Laboratories, U.S.A.)
NMR ¹H spectra of products were recorded with a Bruker 400 MHz spectrometer in deuterium oxide.

### 1.1.2. Used Equipment

1. Rheometer Kinexus Ultra+, NETZSCH
2. Pressure controller: Microfluidic flow controller Flow EZ™, Fluigent
3. Airtight metal tube caps for microfluidics P-CAP 1.5 mL, Fluigent

### 1.1.3. MKN45 Cell Medium

Roswell Park Memorial Institute medium (RPMI) 1640, 1× GlutaMAX, 20% of

FBS (Fetal Bovine Serum)
1. RPMI Medium 1640+GlutaMAX, (Gibco, 6187-010)
2. Fetal Bovine Serum Qualified One Shot, (Gibco, A3161001)

### 1.1.4. IGROV1 Cell Medium

RPMI 1640, 1× GlutaMAX, 10% of FBS
1. RPMI Medium 1640+GlutaMAX, (Gibco, 6187-010)
2. Fetal Bovine Serum Qualified One Shot, (Gibco, A3161001)

### 1.1.5. HEP 3B Cell Medium

Minimum Essential Medium (MEM) alpha, 1% Non-Essential Amino Acids, 10% FBS, 1× GlutaMAX
1. MEM alpha+GlutaMAX, (Gibco, 32561037)
2. Non-Essential Amino Acids, Minimum Essential Medium Non-Essential Amino acids (MEM NEAA) (100X), (Gibco, 11140-050)
3. Fetal Bovine Serum Qualified One Shot, (Gibco, A3161001)
   1.1.5. Bis. MDA-MB-468 Cell Medium
Dulbecco's Modified Eagle Medium/Nutrient Mixture F-12, 10% FBS
1. DMEM/F-12+GlutaMAX, (Gibco, 10565018)
2. Fetal Bovine Serum Qualified One Shot, (Gibco, A3161001)
   1.1.5. Ter. H1975 Cell Medium
Dulbecco's Modified Eagle Medium/Nutrient Mixture F-12, 10% FBS
1. DMEM/F-12+GlutaMAX, (Gibco, 10565018)
2. Fetal Bovine Serum Qualified One Shot, (Gibco, A3161001)

### 1.1.6. Encapsulation Buffer

RPMI 1640, 1× GlutaMAX, 100 mM HEPES, 10% FBS, adjusted to pH 7.6 using 1 M NaOH and 1M HCl.
1. RPMI Medium 1640+GlutaMAX, (Gibco, 6187-010)
2. Fetal Bovine Serum Qualified One Shot, (Gibco, A3161001)
3. HEPES (1M), (Gibco, 15630-080)

### 1.1.7. Preparation of Cells to be Encapsulated

a. MKN45 cells preparation to encapsulation
MKN-45 is a human gastric cancer cell line derived from poorly differentiated gastric adenocarcinoma.

The process for the preparation of MKN-45 cells comprises at least the following steps:
1) Taking the flask with the cells
2) Keeping the supernatant
3) Adding 1 mL of accutase to cell culture flask T25, 2 mL to cell culture flask T75
4) Keeping 10-15 min until the detachment of the cell at 37° C.
5) Adding 5× volume of accutase fresh cell medium containing FBS and transfer cells solution to the kept supernatant
6) Centrifuging at 300 g for 5 min
7) Removing the supernatant
8) Diluting cells at desired concentration in encapsulation buffer
9) Filtering them through 37 μm strainer 10) Using immediately for the preparation of encapsulation solution
   b. IGROV1, HEP3B, MDA-MB-468, and H1975 Cells Preparation to Encapsulation
The IGROV-1 cell line is a human ovarian carcinoma cell line.
HEP3B cell line is a human hepatoma cell line.
MDA-MB-468 cell line is a human breast adenocarcinoma cell line.
H1975 cell line is a human non-small cell lung carcinoma cell line.

The process for the preparation of IGROV1, HEP3B, MDA-MB-468 or H1975 cells comprises at least the following steps:
1) Taking the flask with the cells
2) Removing the supernatant
3) Rinsing with at least 5 mL of PBS for cell culture Flask T25-T75
4) Adding 1 mL of trypsin to cell culture flask T25, 2 mL to cell culture flask T75 5) Keeping 10-15 min until the detachment of the cell at 37° C.
6) Adding fresh cell medium containing FBS, 5× volume of trypsin used and transfer cells solution to the centrifuge tube
7) Centrifuging at 300 g for 5 min
8) Removing the supernatant
9) Diluting cells at desired concentration in encapsulation buffer
10) Filtering them through 37 μm strainer
11) Using immediately for the preparation of encapsulation solution
   1.1.8. Preparation of Dextran-VS 70 kDa. DS=33% and 22%
   a. Preparation of Dextran-VS 70 kDa, DS=33%
   Modification of Dextran by DVS with DM (Degree of Modification, Also Named Degree of Substitution DS)=33%:

A 2% w/v solution of dextran was prepared by dissolving 8 g of dextran in 400 mL of [0.1 M] NaOH solution. A quantity of 19.2 mL of divinyl sulfone (1.5 equivalents compared to hydroxyl groups of the polymer) was added instantly into the vigorously vortexed polymer solution. After 3 minutes, the reaction was stopped by adjusting the pH to 5 with 5 M HCl. Then the vinyl sulfone modified dextran was purified by dialysis using MWCO-3.5 KD membrane bags against acidified water (HCl, pH=5) for 24 h and then against pure water at pH=7 for other 4 days, with two changes of water per day. The dialyzed solution was filtered through a 5 μm pore filter, and finally freeze-dried. The desired compound was characterized by ¹H NMR in D₂O.

The degree of modification (DM) of the dextran by the vinyl-sulfone groups was calculated by the ratio of integrated ¹H NMR signals of the vinyl protons of the vinyl sulfone groups compared to the proton at position 1 of the dextran monomer.

b. Preparation of Dextran-VS 70 kDa, DS=22%
Modification of Dextran by DVS with DM (Degree of Modification, Also Named Degree of Substitution DS)=22%:

A 2% w/v of dextran were prepared by dissolving 8 g of dextran in 400 mL of [0.1 M] NaOH solution. A quantity of 19.2 mL of divinyl sulfone (1.5 equivalent to hydroxyl groups of the polymers) was added instantly into the vigorously mixing polymer solution. After 1.5 minutes, the reaction was stopped by adjusting the pH to 5 with 5 M HCl. Then the vinyl sulfone modified dextran was purified by dialysis using MWCO=3.5 KD membrane bags against acidified water (HCl, pH=5) for 24 h and then against pure water at pH=7 for other 4 days, with two changes of water per day. The dialyzed solution was filtered through a 5 μm pore filter, and finally freeze-dried. The desired compound was characterized by ¹H NMR in D₂O.

The degree of modification of the dextran by the vinyl-sulfone groups was calculated by the ratio of integrated ¹H NMR signals of the vinyl protons of the vinyl sulfone groups compared to the proton at position 1 of the dextran monomer.

RMN ¹H in D20:7.0-6.8 ppm (m, ¹H, Ha-vinyl): 6.5-6.3 ppm (dd, 2H, Hb-vinyl): 5.25-4.9 ppm (s, ¹H, H1-dextran): 4.2-3.1 ppm (m, nH, H2,3,4,5,6,6′ dextran+CH₂-vinyl)

### 1.1.9. Description of the General Method for Cell Encapsulation in Dextran-VS—SH-PEG-SH-VS-Dextran Hydrogel Gel-Steps (a) to (d)

5m of collection tubing were prepared, and then it was wrapped around an aluminum bar which temperature was controlled by Peltier's element. The tube was prefilled with a pure HFE7500 oil.

The Peltier's element was preheated to 37° C.

The corresponding volume of the cell solution in encapsulation buffer was taken and the corresponding volume of the Dextran-VS solution was added, to formulate one of two equal volume water phases used in droplet production

The corresponding volume of SH-PEG-SH, was taken and was added to the corresponding volume of encapsulation buffer, to formulate one of two equal volume water phases used in droplet production

The droplets using 3 enter microfluidic chip (two enters for equal volume water phases, comprising cells in encapsulation buffer with Dextran-VS and SH-PEG-SH in encapsulation buffer, and one enter for HFE7500 with 2.5% (w/v) of fluorosurfactant) were produced, collected through the collection tube kept at 37° C., and collected to the protein low-bind tube.

After the end of encapsulation, the collection tube with the help of pure HFE7500 oil was slowly emptied.

The droplets were kept at least 15 min at 37° C.

1.1.10. Description of the General Method for the Preparation of Hydrogel Dextran-VS-SH-PEG-SH-VS-Dextran Beads by Extraction from the HFE7500 Oil with RAN Surfactant-Step (e)

The beads were washed at least once with the 2× volume of emulsion fresh (without surfactant) HFE7500 oil.

The beads were centrifuged at 100 g for 10 s and the rest of oil was removed.

3× emulsion volume of corresponding cell medium or another aqueous phase was added.

2× emulsion volume of ¹H, ¹H,2H,2H-perfluoro-1-octanol was added, and then it was waited till the beads go up to aqueous phase.

¹H, ¹H,2H,2H-perfluoro-1-octanol was removed from the bottom of the tube, using pipette or syringe with attached needle.

The tube was centrifuged at 100 g for 20s, and the rest of the ¹H, ¹H,2H,2H-perfluoro-1-octanol was removed.

The procedure above was repeated until to obtain a homogeneous phase.

Beads were transferred in aqueous solution to the T25 non-adherent cell culture flask (for suspension cell culture) with cell medium.

1.1.11 Passage of Dextran-VS-SH-PEG-SH-VS-Dextran Beads with Encapsulated Cells-Step (f)

Cell media with beads were taken from the flask and were put to protein low-bind tube. Then, this was centrifuged at 30 g for 3 min.

The medium was removed with serological pipette.

The fresh cell medium was added. The beads were transferred to the flask. The flask was put in the incubator, at 37° C., 5% CO₂.

### 1.1.12. Preparation of a Solution of 20% (w v) Dextran-VS

Dextran-VS powder prepared according to 1.1.8 was dissolved in deionized water. To obtain 20% (w/v) of Dextran-VS solution 100 mg of Dextran-VS were added to 444 μL of water.

### 1.1.13. Preparation of 30% (w v) SH-PEG-SH 1.5 kDa Solution

SH-PEG-SH powder was dissolved in deionized water. To obtain 30% (w/v) of SH-PEG-SH solution, 100 mg of SH-PEG-SH were added to 253 μL of water.

### 1.2. Mkn45 Spheroids Production

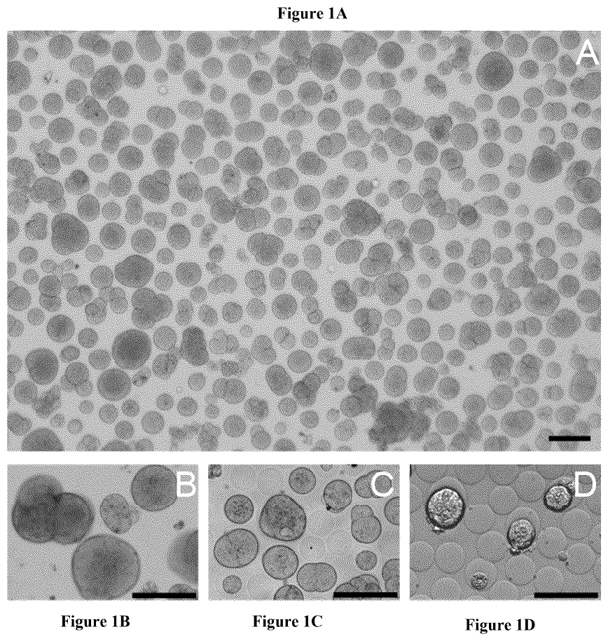

Before the encapsulation and after the defrosting, the MKN45 cells were submitted to at least 3 passages. More particularly, the MKN45 cells were passed twice a week at 0.04M/mL in MKN45 cell medium. The cells were encapsulated at exponential phase of growth according to the General Method as described above in paragraph 1.1.9. After the encapsulation, the dextran beads encapsulating the cells were passed every two days and then from the 5th day onwards, every day according to the procedure as described above in paragraph 1.1.11. A 2.8% (w/v) gel was used. To obtain 300 μL of this gel, 31.25 μL of Dextran-VS (DS=22%) at 20% (w/v) prepared according to paragraph 1.1.12. above, 7.17 μL of SH-PEG-SH at 30% (w/v) prepared according to paragraph 1.1.13. above and 261.6 μL of the encapsulation buffer with cells were added. The size of the thus produced droplets was 75 μm and the cell concentration at encapsulation was 4M/mL. The spheroids obtained from the MKN45 cells are shown in FIG. 1A.

Surprisingly, the inventors have discovered that the thus obtained solid spheroids were formed without any preliminary cell sorting or changing the culture conditions as it was shown in some previous articles such as:
Jianming, L., et al., Spheroid body-forming cells in the human gastric cancer cell line MKN-45 possess cancer stem cell properties. International Journal of Oncology, 2013:
Meritxell, B., et al., Analysis of the Effect of Increased α2,3-Sialylation on RTK Activation in MKN45 Gastric Cancer Spheroids Treated With Crizotinib. International Journal of Molecular Sciences, 2020:
Özlem, T. and T. Gökhan, Effects of growth factor deprivation on MKN-45 spheroid cells. Turkish journal of biology=Turk biyoloji dergisi, 2023; and
Shigeo, T., et al., Identification of Gastric Cancer Stem Cells Using the Cell Surface Marker CD44. Stem Cells, 2009.

In addition, it comes out that the thus obtained spheroids according to the present disclosure possess higher quality than spheroids from MKN45 cell line in ULA-plate (Ultra-Low Attachment-plate) conditions or in 3D Petri Dish® as the latter form loose aggregates and do not form a tight spheroid body (Meritxell, B., et al., Multicellular Human Gastric-Cancer Spheroids Mimic the Glycosylation Phenotype of Gastric Carcinomas. Molecules, 2018). Further, with the latter previously known spheroids, it was impossible to dispense them and to transfer them with a pipette.

The method of MKN45 spheroids production described above could be repeated with different substitution degrees of Dextran-VS. To demonstrate it, MKN45 cells were prepared and encapsulated as described above in the gel of following composition: 27.82 μL of Dextran-VS (DS=33%) at 20% (w/v) prepared according to paragraph 1.1.12. above, 9.45 of SH-PEG-SH at 30% (w/v) prepared according to paragraph 1.1.13 above and 262.73 μL of encapsulation buffer with cells. The spheroids obtained from MKN45 cells, encapsulated this gel are shown on FIG. 7.

### 1.3. Hep3B Spheroids Production

Before the encapsulation and after the defrosting, the HEP3B cells have to be submitted to at least 2 passages. More particularly, the HEP3B cells were passed twice a week at 0.2M/mL in HEP3B cell medium. The cells were encapsulated at exponential phase of growth according to the General Method as described above in paragraph 1.1.9. After the encapsulation, the dextran beads encapsulating the cells were passed every two day and then from the 10th day onwards, every day according to the procedure as described above in paragraph 1.1.11. A 2.8% (w/v) gel was used, and encapsulation of small aggregates of the cells was carried out. To obtain 300 μL of this gel, 31.25 μL of Dextran-VS (DS=22%) at 20% (w/v), prepared according to paragraph 1.1.12. above, 7.17 μL of SH-PEG-SH at 30% (w/v) prepared according to paragraph 1.1.13. above and 261.6 μL of the encapsulation buffer with cells were added. The size of the thus produced droplets was 75 μm and the cell concentration at encapsulation was 5M/mL. The spheroids obtained from the HEP3B cells are shown in FIG. 1B.

This cell line naturally forms spheroids under Ultra-Low Attachment (ULA) plate conditions (Rodríguez-Hernández, M. A., et al., Differential effectiveness of tyrosine kinase inhibitors in 2D/3D culture according to cell differentiation, p53 status and mitochondrial respiration in liver cancer cells. Cell Death & Disease, 2020. 11 (5): p. 339). The process described in the present disclosure can replicate this behavior, suggesting the potential for universal application. This indicates that a wide variety of spheroids can be produced. Moreover, the inventors have noted that the resulting spheroids exhibit a remarkably high level of manipulability, and the total number of spheroids produced in a single experiment exceeds that achieved using ULA plate.

### 1.4. Igrov1 Spheroids Production

Before the encapsulation and after the defrosting, the IGROV1 cells were submitted to at least 2 passages. More particularly, the IGROV1 cells were passed twice a week at 0.2M/mL in IGROV1 cell medium. The cells were encapsulated at exponential phase of growth according to the General Method as described above in paragraph 1.1.9. After the encapsulation, the dextran beads encapsulating the cells were passed every two days and then from the 10th day onwards, every day according to the procedure as described above in paragraph 1.1.11. A 3.5% (w/v) gel was used. To obtain 300 μL of this gel, 39.07 μL of Dextran-VS (DS=22%) at 20% (w/v) prepared according to paragraph 1.1.12. above, 8.96 μL of SH-PEG-SH at 30% (w/v) prepared according to paragraph 1.1.13 above and 252 μL of the encapsulation buffer with cells were added. The size of the thus produced droplets was 75 μm and the cell concentration at encapsulation was 5M/mL. The spheroids obtained from the IGROV1 cells are shown in FIG. 1C.

Tight spheroids from IGROV1 cells were obtained. Before this experiment it was shown and known that this cell line is capable to form only loose aggregates (Anaïs, W., et al., Evaluation of the potential of a new ribavirin analog impairing the dissemination of ovarian cancer cells, PLOS ONE, 2019; Nina, H., et al., ADAM17 Inhibition Increases the Impact of Cisplatin Treatment in Ovarian Cancer Spheroids. Cancers, 2021: Sabrina, K., et al., Initial formation of IGROV1 ovarian cancer multicellular aggregates involves vitronectin. Tumor Biology, 2010, and Salvatore, C., et al., B-Catenin-regulated ALDH1A1 is a target in ovarian cancer spheroids. Oncogene, 2015), these loose aggregates could not be pipetted or transferred without causing damages, and morphologically do not represent spheroids. Now, the inventors have proven that the process according to the present disclosure allows a surprisingly high manipulability and spheroid's morphology.

### 1.5. Mda-Mb-468 Spheroids Production

Before the encapsulation and after the defrosting, the MDA-MB-468 cell have to be submitted to at least 3 passages. More particularly, the MDA-MB-468 cells were passed twice a week at 0.2M/mL in MDA-MB-468 cell medium. The cells were encapsulated at an exponential phase of growth according to the General Method as described above in paragraph 1.1.9. After the encapsulation, the dextran beads encapsulating the cells were passed every two days according to the procedure as described above in paragraph 1.1.11. A 3.3% (w/v) gel was used. To obtain 300 μL of this gel, 36.83 μL of Dextran-VS (DS=22%) at 20% (w/v) prepared according to paragraph 1.1.12. above, 8.44 μL of SH-PEG-SH at 30% (w/v) prepared according to paragraph 1.1.13. above and 254.72 μL of the encapsulation buffer with cells were added. The size of the thus produced droplets was 75 μm and the cell concentration at encapsulation was 4M/mL. The spheroids obtained from the MDA-MB-468 cells are shown in FIG. 1D.

That is known that spheroids were obtained with MDA-MB-468 cell line in Ultra-Low Attachment (ULA) plate condition. This result confirms that the process according to the present disclosure may be applied to a wide range of cell lines, underlying its universality (Ziperstein, M. J., A. Guzman, and L. J. Kaufman, Breast Cancer Cell Line Aggregate Morphology Does Not Predict Invasive Capacity. PLOS One, 2015. 10 (9): p. e0139523).

1.5 Bis. H1975 Spheroids Production

Before the encapsulation and after the defrosting, the H1975 cell have to be submitted to at least 2 passages. More particularly, the H1975 cells were passed twice a week at 0.15M/mL in H1975 cell medium. The cells were encapsulated at an exponential phase of growth according to the General Method as described above in paragraph 1.1.9. After the encapsulation, the dextran beads encapsulating the cells were passed every two days according to the procedure as described above in paragraph 1.1.11. A 3.1% (w/v) gel was used with the adding of 1% of Matrigel™. To obtain 300 μL of this gel, 32.59 μL of Dextran-VS (DS=22%) at 20% (w/v) prepared according to paragraph 1.1.12. above, 9.27 μL of SH-PEG-SH at 30% (w/v) prepared according to paragraph 1.1.13. above and 249.13 μL of the encapsulation buffer with cells were added as well as 3 μL of the Matrigel™ that was added into the solution for the encapsulation with the SH-PEG-SH phase. The size of the thus produced droplets was 75 μm and the cell concentration at encapsulation was 4M/mL. The spheroids obtained from the H1975 cells are shown in FIG. 12.

### 1.6. Measuring Shear Modulus of the Dextran-Vs (DS=22%)-SH-PEG-SH Gel

As mentioned above, Young's modulus and shear modulus are related by E=2G (1+v) (for isotropic and homogeneous materials), E is Young's modulus, G is shear modulus and v is Poisson's ratio.

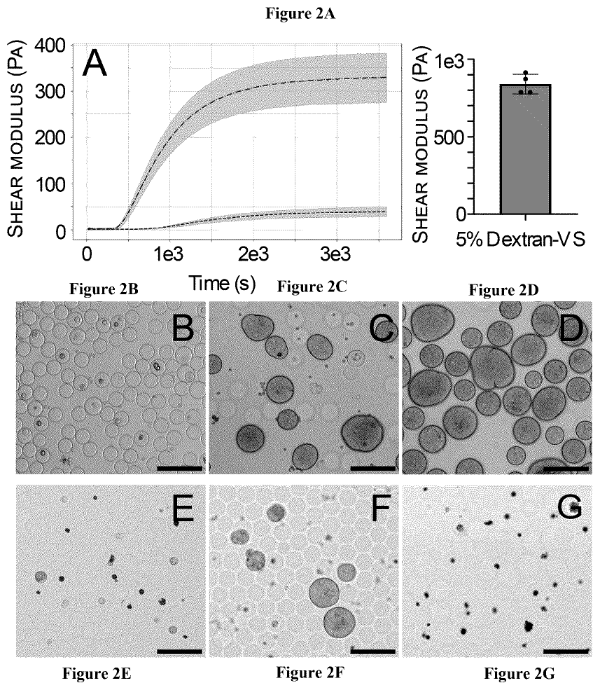

Shear modulus of various gels as obtained in examples 1.2 to 1.5 above at 2.8% (w/v) and 3.5% (w/v) were measured during their polymerization, briefly, all solutions were prepared on ice, and rheometer pad and geometry were cooled to 2° C. A droplet of liquid gel was positioned in the center of cooled padding. Rheometer geometry pulled down to form 100 μm height and 2 cm large disk of liquid gel. After that the padding was heated to 15° C. and the measurement were taken until the polymerization plateau. For the 5% (w/v) Dextran gel the protocol is the same, except that measurements were taken just after the polymerization during 10 min at the pad adjusted to 25° C. All the measurements were made in linear elastic region. Obtained shear modulus shown on FIG. 2A. As could be constituted from the FIG. 2A, bigger gel percentage corresponds to bigger shear modulus.

It is known that different cell lines need different culture conditions and different gel stiffness to efficiently form spheroids. It has been stated by the inventors that the stiffness of the beads obtained in examples 1.2 to 1.5 above may be easily modified, without any loose in simplicity of the protocol. On FIG. 2B-G are shown spheroids from MKN45 and IGROV1 cells obtained at different stiffness of the beads. It is observed that the biggest spheroids formed from MKN45 cell at 2.8% (w/v) gel (FIG. 2D), and 10% (w/v) gel (FIG. 2B) is too stiff for this cell line since there is no spheroid formation. IGROV1 cells form spheroids in 3.5% (w/v) gel (FIG. 2F), and softer (2.8% (w/v) for example as shown in FIG. 2G) and stiffer (5% (w/v) for example as shown in FIG. 2E) gels are not compatible with these cell line. In this example, gels for IGROV1 cell line were formed with the addition of SH-RGD at 100 μM, that later were shown excessive for this cell line. Encapsulations in 3.3% gel, 5% gel, and 10% gel were performed according to examples 1.2 and 1.4 for the corresponding cell lines with the following gels formulations:

| | Dextran-VS (DS = | SH-PEG-SH at 30% | |
|---|---|---|---|
| | 22%), at 20% (w/v) | (w/v) prepared | |
| | prepared according | according to | encapsulation |
| Gel %, | to paragraph 1.1.12 | paragraph 1.1.13 | buffer with |
| (w/v) | (μL) | (μL) | cells (μL) |
| 3.3% | 36.83 | 8.44 | 254.72 |
| 5% | 55.81 | 12.8 | 231.4 |
| 10% | 111.61 | 25.59 | 162.8 |

For encapsulations in 2.8% gel and 3.5% gel, the details of the used quantities of Dextran-VS (DS=22%) at 20% (w/v), SH-PEG-SH at 30% (w/v) and encapsulation buffer with cells are mentioned above in examples 1.2 and 1.4.

1.7. EC50 Definition with MKN45 Spheroids

Spheroids produced in examples 1.2 to 1.5 can be directly used in drug testing. More particularly, the MKN45 spheroids prepared in example 1.2 were used in this example for the drug testing with ADC (antibody drug conjugate)-approx. 150 000 Da representing large molecule and MEKi (MEK inhibitor), representing example of small molecule, approximately 482 Da. One of the common technics is the usage of CellTiter-Glo® 2.0 Cell Viability Assay. Briefly, equal number of spheroids distributed in well plate using sample pipette with different concentration of the tested drug. At a given time CellTiter-Glo® 2.0 Cell Viability Assay is added, and it lyses the spheroids and liberates cellular ATP. Read out is a luminescent signal generated by components of CellTiter-Glo® 2.0 Cell Viability Assay reacting with cellular ATP.

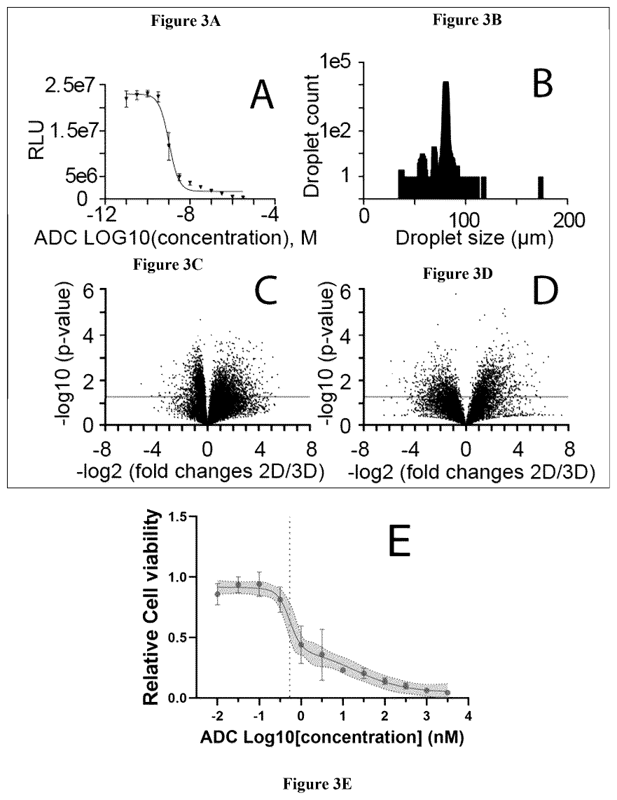

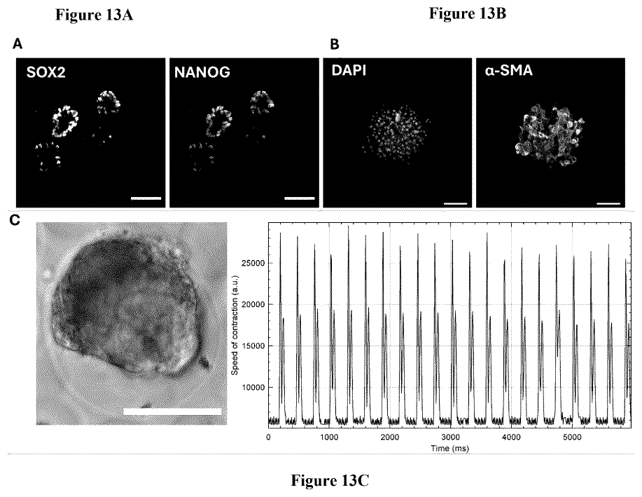

On FIG. 3A, FIG. 3D and FIG. 11 are shown obtained curves of EC50 test. Interestingly spheroids are always protected by gel shell, that allows to pipette them, centrifuge and distribute them in a well plate without a risk of their damage. It has further been demonstrated that lysis of spheroids and ATP-sensitive reaction can be performed directly on spheroids protected by a gel shell, allowing to decrease the number of manipulation and allow avoiding the undesired concomitantly action of dextranase with the component of CellTiter-Glo® 2.0 Cell Viability Assay or with the drug, and it shows that spheroids in hydrogel shell are accessible for the drug tests as well with the small molecules or with large ones.

### 1.8. Droplet Size

Cells encapsulation in monodisperse droplets results in monodispersed hydrogel beads with cell. Optimum polymerization speed and viscosity of the components of Dextran-VS-SH-PEG-SH gel allow to produce beads with Coefficient of Variation (CV) equal to 1.3%. On the FIG. 3B is presented the size analysis of 200 000 droplets during their production. Laser beam was used, that scans passing by microfluidic channel droplets, knowing the frequency of droplet production and water phase flows allowing to calculate the size of the droplets.

Data obtained for this example is a laser scan of the droplets produced during the encapsulation of MKN45 cells like described in 1.2 above.

### 1.9. RNA SEQUENCING (RNASEQ) OF MKN45 and IGROV1 SPHEROIDS

### Used Materials:

1. RNeasy Mini Kit, (QIAGEN, 74136)
2. RNA concentration:
   a. Qubit 4 Fluorometer, (ThermoFisher)
   b. Qubit RNA, Broad Range (Invitrogen, Q10210)
3. Quality check RIN:
   a. 4200 Tape Station, (Agilent, G2991A)
   b. High Sensitivity RNA ScreenTape (Agilent, 5067-5579)
   c. High Sensitivity RNA ScreenTape Sample Buffer (Agilent, 5067-5580)
   d. High Sensitivity RNA ScreenTape Ladder (Agilent, 5067-5581)

The RNA extraction protocol from MKN45 and IGROV1 spheroids which were produced according to the present disclosure (Example 1.2 and Example 1.4) is as follows:
1. Three T25 non-adherent cell flasks were treated with 5 mL of poly-d-lysine at 37° C. for 30 min
2. Poly-d-lysine was discarded from flasks
3. Flasks was washed with 10 mL of DPBS
4. Spheroids in cell culture medium were transferred to the first treated flask and left at 37° C. for 15 min
5. Spheroids were transferred to the second treated flask and left at 37° C. for 15 min
6. Spheroids were transferred to the third treated flask and left at 37° C. for 15 min
7. Spheroids were transferred to the 15 mL Protein low-bind tube
8. Spheroids were centrifuged for 5 min at 100 g
9. Supernatant was discarded
10. Spheroids were resuspended in 1 mL of cold DPBS
11. 5 μl of Dextranase was added
12. Spheroids were centrifuged for 5 min at 100 g at 4° C.
13. Supernatant was discarded, 12 mL of fresh cold DPBS added
14. After that QIAGEN Rneasy protocol of RNA extraction was used, according to the kit's instractions
15. Quantification of RNA quality and quantity was performed according to instruction with 4200 Tape Station and Qubit 4 Fluorometer
16. Samples with RIN>9 were sent to sequencing services provider and analyzed using standard approaches.

RNA extraction from 2D MKN45 and 2D IGROV1 cells, was performed according to standard protocol RNeasy Mini Kit, and further analysis and quantification were performed in the same way that RNA originated from MKN45 and IGROV1 spheroids.

The present data demonstrate that spheroids produced from MKN45 and IGROV1 cells (see Examples 1.2 and 1.4 above), treated with dextranase and processed according to the described protocol, are compatible with RNA extraction for subsequent RNA sequencing analysis.

FIGS. 3C and 3D clearly illustrate significant differences in gene expression profiles between spheroid and standard 2D culture conditions. Specifically, the volcano plots in FIG. 3C reveal distinct gene expression changes in MKN45 cells when grown as spheroids compared to their 2D counterparts, highlighting the impact of three-dimensional culture on cellular transcriptional activity. Similarly, FIG. 3D shows the gene expression differences in 14-day-old IGROV1 spheroids compared to 2D cultured cells, further confirming that spheroid culture significantly alters the cellular transcriptome.

The compatibility of these spheroids with RNA extraction and RNA sequencing also facilitates their use in advanced genomic studies, paving the way for more detailed and accurate molecular characterizations in cancer research.

### 1.10. Immunostaining of Spheroids

### Primary Antibodies:

1. Anti-Ki67, Rabbit, (Abcam, AB15580-1001), dilution 1/700
2. Anti-e-Cadherin, Mouse, (BD transduction laboratories, 61082), dilution 1/250

### Secondary Antibodies:

1. Anti-Rabbit, Donkey, Alexa-488 (Invitrogen, A21206), dilution 1/500
2. Anti-Mouse, Donkey, Alexa-594 (Invitrogen, A32744), dilution 1/500

### Other Staining

1. DAPI (Thermo Fisher, D9542)
2. Phalloidin, Alexa Fluor Plus 647, (Thermo Fisher, A22287)

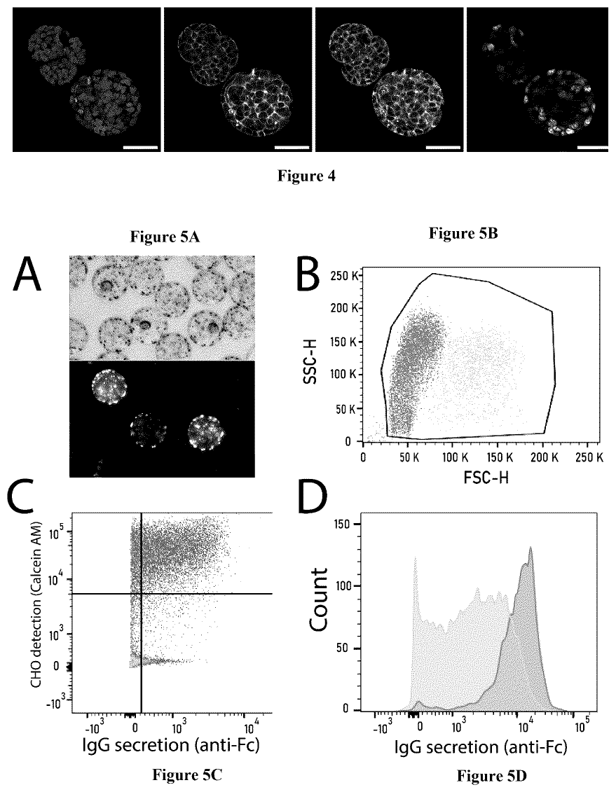

Spheroids in accordance with the present disclosure were fixed using known protocol (Dekkers, J. F. et all., Nature protocols, 2019, High-resolution 3D imaging of fixed and cleared organoids, 14 (6), 1756-1771). with the difference, that fixation of spheroids by PFA (paraformaldehyde) was performed before gel dissolution, and dissolution of the gel was made by 1/2000 solution of dextranase in 0.1% (v/v) PBS-Tween buffer right after the fixation. Example of the immunofluorescence (IF) imaging of IGROV1 spheroid is shown on FIG. 4. As could be observed from the images, spheroids obtained according to 1.4 present developed E-cadherin patterns and are compatible with IF methods.

### Example 2: High-Throughput Screening of Igg Secreting Cells and Sorting of Highest Igg Producer Cells

### 2.1. Material and Methods

### 2.1.1. Used Products

1. 1H,1H,2H,2H-perfluoro-1-octanol (Sigma, ref. 370533)
2. 3M™ Novec™ 7500 Engineered Fluid (2-trifluoromethyl-3-ethoxydodecafluorohexane), called HFE7500, 3M
3. 37 μm Reversible Strainer, (Stemcell, 27215)
4. Protein LoBind Tube 1.5 mL (Eppendorf, 022431081)
   a. Protein LoBind Conical Tubes 15 mL (Eppendorf, 0030122216)
   b. Protein LoBind Conical Tubes 50 mL (Eppendorf, 0030122240)
5. HEPES (1M), (Gibco, 15630-080)
6. SH-PEG-SH (PEG dithiol) 1.5 kDa, (Merck, JKA4105-1G)
7. Dextran-VS (Dextran-Vinyl sulfone) 70 kDa, DS-22% (degree of substitution)
8. 300 nm Magnetic Streptavidin Beads (Ademtech, ref. 03131)
9. Biotin Vhh anti-Human Kappa (ThermoFisher, ref. 7103272500)
10. Biotin Vhh anti-Murine Kappa (ThermoFisher, ref. 7103152500)
11. Medium CD CHO (ThermoFisher, 10743029)
12. Pluronic F68 (ThermoFisher, 24040032)
13. Fluorescent antibody anti-Human IgG Fc Fragment, Alexa 647 (Jackson ImmunoResearch, ref. 309-006-008)
14. Calcein AM, (ThermoFisher, C1430)

### 2.1.2. Used Equipments

1. BD FACS Aria™ Fusion Flow Cytometer
2. Microfluidic Station

### 2.1.3. CHO Encapsulation Buffer Medium CD CHO.

### 2.1.4. CHO Cells (Chinese Hamster Ovary Cells) Preparation to Encapsulation

Chinese hamster ovary cells are a family of immortalized cell lines derived from epithelial cells of the ovary of the Chinese hamster.

The process for the preparation of CHO cells comprises at least the following steps:
1) Taking the flask with the CHO cells
2) Centrifuging at 300 g for 5 min
3) Removing the supernatant
4) Diluting cells at desired concentration in CHO encapsulation buffer
5) Filtering them through 37 μm strainer
6) Using immediately for the preparation of encapsulation solution
   2.2. Magnetic Beads Preparation for Catching Human IgG Magnetic beads were prepared for catching human IgG by implementing the following steps:
1. Taking 60 μL of bead Streptavidin for 100 μL of final solution.
2.Putting off the supernatant using magnet.
3. Adding 6 μL of VHH anti-human Kappa.
4. Incubating at least for 45 min at RT
5. Putting off the supernatant using magnet.
6. Washing beads with 90 μL DPBS (Dulbecco Phosphate Buffered Saline) twice.
7. Resuspending in 100 μL final buffer.
8. Just before every encapsulation: Vortex beads, sonicating the beads: 1 min on “sweep” 37 kHz 80% Power and 1 min on pulse 80 KHz 80% P in ice and vortex again

### 2.3. Cell and Beads Encapsulation in Dextran-VS-Sh-Peg-SH-VS-DEXTRAN HYDROGEL GEL-Steps (a) to (d)

The Peltier's element was preheated to 37° C. The corresponding volume of the cell solution in CHO encapsulation buffer was taken and the corresponding volume of the Dextran-VS solution was added.

The corresponding volume of SH-PEG-SH prepared according to example 1.1.13 was taken and was added to the corresponding volume of CHO encapsulation buffer containing the capture Beads.

The droplets using 3 enters-microfluidic chip were produced, collected through the collection tube kept at 37° C., and collected to the protein low-bind tube. The droplets were kept at least 30 mins at 37° C.

2.4. Hydrogel Dextran-Vs (Ds=22%)-Sh-Peg-Sh-Vs-Dextran Beads Extraction from the Hfe7500 Oil with Ran SURFACTANT-Step (e)

The beads were washed at least once with the 2× volume of emulsion fresh (without surfactant) HFE7500 oil.

The beads were centrifuged at 100 g for 10s and the rest of oil was removed.

3× emulsion volume of corresponding cell medium or another aqueous phase with 1% Pluronic F68 was added. 2× emulsion volume of 1H, 1H,2H,2H-perfluoro-1-octanol was added, and then it was waited till the beads go up to aqueous phase.

1H, 1H,2H,2H-perfluoro-1-octanol was removed from the bottom of the tube, using pipette or syringe with attached needle. The tube was centrifuged at 100 g for 20s, and the rest of the 1H, 1H,2H,2H-perfluoro-1-octanol was removed.

The procedure above was repeated as above if any nonhomogeneous phase was seen. The beads are then transferred in aqueous solution.

### 2.5. Single Cell Cho Human Igg Secretion Detection

CHO cell should have a good viability (>90%) before the encapsulation. The CHO cells were modified to secret Human IgG. Good results were obtained with 5% (w/v) gel. To prepare 200 μL of emulsion a first solution of 100 μL with 29.8 μL of Dextran-VS (DS=22%) at 20% (w/v) prepared according to paragraph 1.1.12 and 70.21 μL of CHO encapsulation buffer was used, and a second solution of 100 μL with 6.8 μL of SH-PEG-SH at 30% (w/v) prepared according to paragraph 1.1.13 and 10 μL of Pluronic F68 and 83.2 μL of CHO encapsulation buffer was used. The cells were resuspended in the Dextran solution at a desired concentration (ex: 10 million cells/mL). The magnetic beads were resuspended in the SH-PEG-SH solution (see final buffer). The droplet size was adjusted at 40 μm. After beads extraction from the emulsion, the beads solution was kept at 4° C. to avoid CHO IgG secretion. To reveal the IgG secretion, a fluorescent antibody anti-human IgG Fc Fragment was added to the beads solution and incubated for 30 min at 4° C. The beads were then washed two times with a PBS solution to remove the unbonded fluorescent antibody. To stain the CHO cells, Calcein AM was used. By following the recommendation of the manufacturer, a cell staining was obtained with a green fluorescence. At this point the hydrogel beads were analyzed, for example by a fluorescent microscope (FIG. 5-A) or with a flow cytometer or with Flow Active Cell Sorter (FIGS. 5B, 5C, 5D).

Surprisingly, it was observed that very resolutive dot plots on flow cytometer (FIG. 5B & 5D) were obtained compared to previous works using hydrogel beads (Fang, Y., et al., Going native: Direct high throughput screening of secreted full-length IgG antibodies against cell membrane proteins. MAbs, 2017. 9 (8): p. 1253-1261). This could be explained by two points, first the mono-dispersity of the obtained hydrogel beads due to the low viscosity of Dextran and second due to the high transparency of the dextran hydrogel. Thanks to these, the hydrogel beads with or without cells (FIG. 5B) were very easily distinctly identified and, a very resolutive fluorescent signal was obtained (FIG. 5C & 5D). The capacity to easily discriminate the secretion rate at the single cell level was thus obtained and as well as the capacity to distinguish the high IgG antibody CHO cells producers versus the low IgG antibody CHO cells producers. Thanks to this approach, herein is thus provided a method to select the best IgG producers CHO cells from a pool and sort them.

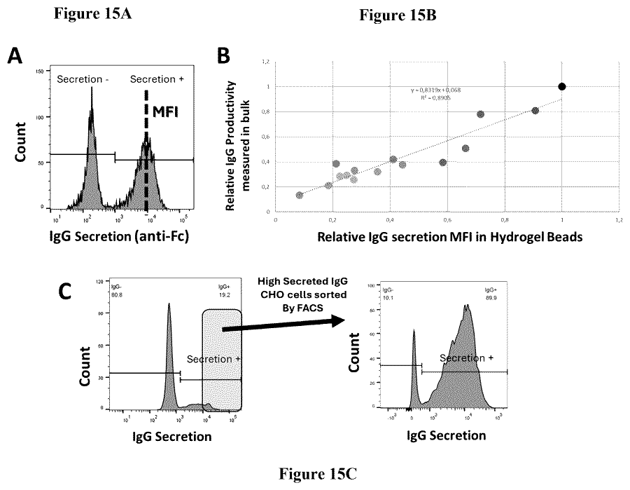

Surprisingly, the inventors have also discovered a strong correlation between the measure of the mean fluorescent intensity of the IgG secretion signal of the positive secreted cell population inside the hydrogel beads as defined in the present disclosure (see FIG. 15A) and the cell IgG productivity measured in bulk culture of the same cell population (see FIG. 15B). These results allow to demonstrate the capacity of the present technology to predict with accuracy the cell IgG productivity (quantity of antibody product by a cell).

Following these results, the inventors have decided to encapsulate in the hydrogel beads as defined in the present disclosure (include the bioassay for IgG detection) a pool of CHO cells with only few cells that well secreted IgG (less than 20%, see FIG. 15C). Thanks to a Flow Active Cell Sorter (FACS) machine, the inventors have sorted only the hydrogel beads as defined in the present disclosure with a CHO cell and with a very high IgG secreted signal (grey rectangular, see FIG. 15C). This sorted CHO cells were then put in culture to expand them. When the inventors have encapsulated these sorted CHO cells in the hydrogel beads as defined in the present disclosure (include the bioassay for IgG detection), they have observed a strong enrichment of the CHO cells secreting IgG (x4.5, see FIG. 15C) compared to the initial pool of CHO cells. This example demonstrates the efficacy of the present technology to select the best secretor cells.

### Material and Methods

### Culture

Frozen cells (around 30E6 (that is to say 30*10⁶ of cells per cryovial) were thawed, transferred to culture medium at an adjusted final cell density of 0.5 viable cells/mL in a E125 shake flask (about 60 mL) and placed in the incubator (5% CO₂, 125 rpm). After 4 days, the cultures were passaging into 60 mL of fresh medium seeded at a seeding density of 0.3E6 vc/mL and placed in the incubator. After 3 days, the culture usually reached VCDs of about 5-8E6 vc/mL, which corresponds to a doubling time of about 15 to 17 h. 20 mL of the cultures were sampled, centrifuged 10 min at 270× g and the pellets were washed twice with ice cold PBS. This step intends to remove antibodies from the cell suspension and ensure that antibody detection following encapsulation and staining measures de novo synthesis/secretion, not carry over from the starting cell culture. The cells were finally resuspended in ice-cold medium at a density of about 10E6 vc/mL and kept on ice for the duration of the encapsulation to stop antibody synthesis/secretion. The remaining of the cell cultures were incubated until harvest for antibody titration.

### Hydrogel Beads Data

Two metrics generated during the FACS analysis were recorded: the % of Alexa 647-positive beads among the calcein-positive beads (this represents the proportion of cells that synthesize and secrete the antibody) and the Alexa 647 MFI (Mean Fluorescence Intensity) measured in calcein-positive beads (this represents the amount of antibody secreted within the secretory population, a surrogate measurement for specific productivity).

### Antibody Titration

Four-day cultures were harvested, submitted to low-speed centrifugation (10 min, 270× g, room temperature) to remove cells and debris. The supernatants were further filtrated through 0.45 μm filters and analyzed by biolayer interferometry on the Octet device.

### Data Analysis

Regarding the % of secretory cells, data generated on different days can be confidently compared due to the clear distinction of the secretory population (beads with secretory cells) among the calcein-positive population (beads with cells). Regarding the MFI, staining may vary from one day to another depending on how the encapsulation medium is prepared, samples handling, staining duration, Alexa-conjugated mAb concentration, among many other factors, despite efforts for standardization. For this reason, MFI raw data, or MFI data normalized to that of an internal control, may be compared across samples processed the same day: however, in order to compare data generated on different days, each experiment should have a common reference sample for data normalization. Similarly, because culture conditions and mAb titration may vary from one experiment to the other, titers were normalized to confidently compare specific productivity across samples.

Five runs were performed on 5 different days with 4 samples per run as follows. The letter in parenthesis (a), (b), (c) and (d) indicates identical samples included in Runs 1-5 so that the results obtained with Runs 2 to 5 can be normalized to that obtained during Run 1.

| | Samples | | | |
|---|---|---|---|---|
| Runs | Sample 1 | Sample 2 | Sample 3 | Sample 4 |
| Run 1 | MCB D0-D0 (a) | MCB D11-D0 (b) | MCB-D32-D0 (c) | MCB-D53-D0 (d) |
| Run 2 | MCB D0-D0 (a) | MCB-D0-D14 | MCB-D0-D28 | MCB-D0-D42 |
| Run 3 | MCB D11-D0 (b) | MCB D11-D14 | MCB D11-28 | MCB D11-42 |
| Run 4 | MCB D32-D0 (c) | MCB D32-D14 | MCB D32-D28 | MCB D32-D42 |
| Run 5 | MCB D53-D0 (d) | MCB D53-D14 | MCB D53-D28 | MCB D53-D42 |

Briefly, in the first normalization step, MFI measurements collected for Sample 2, 3 and 4 were normalized to that of Sample 1 for each individual Run. In the second normalization step, the normalized data sets obtained for Runs 2, 3 and 4, were further normalized to that obtained for Sample 2, 3 and 4 of Run 1, respectively. A similar approach was used to normalize specific productivity data measured during all 5 runs.

Normalized MFI and specific productivity values were then plotted to highlight the correlation between theoretical productivity (MFI measurement) and real productivity (titers measurement).

### Example 3: Gut Organoids Production

### 3.1 Materials and Methods

### 3.1.1 Used Products

1. Matrigel™ (Corning, 56231)
2. LS Columns (Miltenyi Biotec, 130-042-401)
3. QuadroMACS™ Separator (Miltenyi Biotec, 130-090-976)
4. Organoid cell medium (OCM), (M_C0002, Doppl)
5. Collagenase I (17018029, Thermo Fisher Scientific)
6. Collagenase II (17101015, Thermo Fisher Scientific)
7. Hyaluronidase (H3884, Sigma-Aldrich)

### 3.2 Gut Organoids Production-Apical-In Organoids

### 3.2.1. PDSC Preparation-by Company Doppl

Human small intestinal biopsy specimens were rinsed in PBS, and the underlying muscle layers were carefully removed using surgical scissors. The epithelial tissue was minced into about 1-mm fragments and digested enzymatically with collagenase I, collagenase II and hyaluronidase for 30 minutes at 37° C.

After washing, isolated crypts were embedded in 15 μL droplets of Matrigel™ and cultured for seven days. Organoids were then harvested and dissociated into small aggregates using trypsin for 7 minutes at 37° C.

### 3.2.2 PDSC Preparation for Encapsulation

Cell aggregates of PDSC consisting of approximately 5-10 cells (preparation described above in paragraph 3.2.1) were provided by Doppl and subsequently filtered using an LC (Liquid Chromatography) column mounted on a QuadroMACS™ Separator prior to encapsulation. For this process, 1 million cells suspended in 1 mL of OCM were loaded into the column and allowed to pass through by gravity. To recover any remaining cells, the column was washed with an additional 2 mL of OCM. The flow-through was collected in a 15 mL Falcon tube and centrifuged at 200 g for 4 minutes. The resulting cell pellet was then resuspended in 100 μL of OCM to achieve a final concentration of 10 million cells per 1 mL.

### 3.2.3 Dextran and PEG Solution Preparation

An aliquot of Dextran-VS (DS=22.8% solution, prepared at 20% w/v in PBS) and an aliquot of RGD-SH (20 mmol/L in water) were thawed. Subsequently, 33.6 μL of the Dextran-VS solution prepared according to paragraph 1.1.12 above and 13 μL of the RGD-SH solution were combined and briefly vortexed to ensure thorough mixing. The reaction mixture was then incubated at 37° C. for 30 minutes to allow for the covalent modification of Dextran with RGD moieties. Separately, SH-PEG-SH (1.5 kDa, 30% w/v in PBS) prepared according to paragraph 1.1.13 above was thawed on ice prior to use.

### 3.2.4 Encapsulation Solution Preparation

To 83.4 μL of PDSC solution (prepared according to paragraphs 3.2.1 and 3.2.2 above), 46.6 μL of RGD-functionalized Dextran-VS (prepared according to paragraph 3.2.3 above) was added in a Protein LowBind 1.5 mL tube.

In a separate Protein LowBind 1.5 mL tube, 116.9 μL of OCM was combined with 7.9 μL of SH-PEG-SH solution (1.5 kDa, 20% w/v in PBS) and 5.2 μL of Matrigel™.

### 3.2.5 Encapsulation of PDSC

Aggregates of PDSC suspended in the RGD-functionalized Dextran solution (prepared according to paragraph 3.2.3 above)) were encapsulated following the general method as described in paragraph 1.1.9 above, using a mixture of SH-PEG-SH prepared according to paragraph 1.1.13 above and Matrigel solutions.

3.2.6 Recuperation of Hydrogel Beads with PDSC from Oil

The same process as that described in paragraph 1.1.10 above was applied using OCM like a cell medium.

### 3.2.7 Culturing PDSC Encapsulated in Hydrogel Beads

The same procedure as that described in paragraph 1.1.11 above was applied. OCM medium was changed every day.

3.2.8 IF (Immunofluorescence) of Gut Organoids Obtained from PDSC

IF results were provided by Doppl using standard protocol of cell staining.

### 3.3 Gut Organoids Production-Apical-Out Organoids

The procedures for “PDSC preparation,” “PDSC preparation for encapsulation,” “Encapsulation of PDSC,” “Recuperation of hydrogel beads with PDSC from oil,”

“Culturing PDSC encapsulated in hydrogel beads,” and “Immunofluorescence (IF) of gut organoids obtained from PDSC” were performed identically to those previously described for the generation of apical-in organoids in paragraph 3.2 above.

### 3.3.1 Dextran and PEG Solution Preparation

An aliquot of Dextran-VS (22.8% solution, prepared at 20% w/v in PBS) and an aliquot of RGD-SH (20 mmol/L in water) were thawed. Subsequently, 26.9 μL of the Dextran-VS solution prepared according to paragraph 1.1.12 above and 1.3 μL of the RGD-SH solution were combined and briefly vortexed to ensure thorough mixing. The reaction mixture was then incubated at 37° C. for 30 minutes to allow for the covalent modification of Dextran with RGD moieties.

Separately, SH-PEG-SH (1.5 kDa, 20% w/v in PBS) prepared according to paragraph 1.1.13 above was thawed on ice prior to use.

### 3.3.2 Encapsulation Solution Preparation

To 100 μL of PDSC solution (prepared according to paragraphs 3.2.1 and 3.2.2 above), 28.2 μL of RGD-functionalized Dextran-VS (prepared according to paragraph 3.2.3 above) was added in a Protein LowBind 1.5 mL tube.

In a separate Protein LowBind 1.5 mL tube, 119.4 μL of OCM was combined with 6.2 μL of SH-PEG-SH solution (1.5 kDa, 20% w/v in PBS) prepared according to paragraph 1.1.13 above and 2.6 μL of Matrigel™

### 3.4 Results and Conclusions

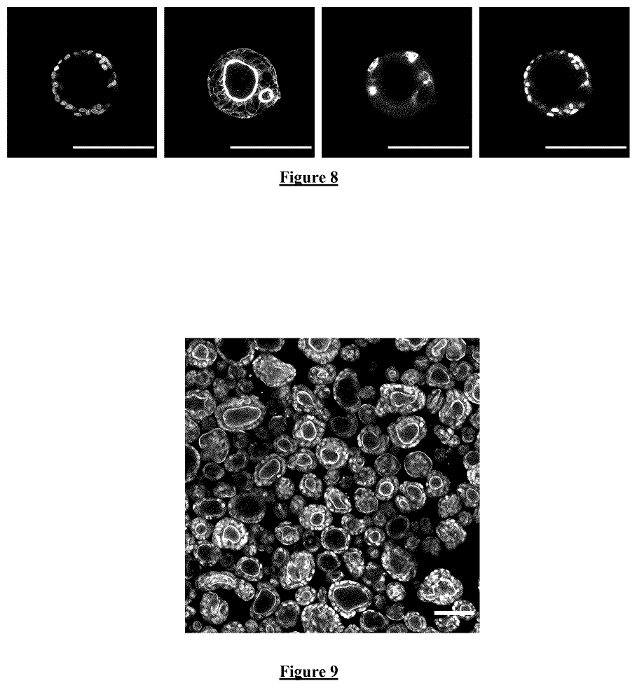

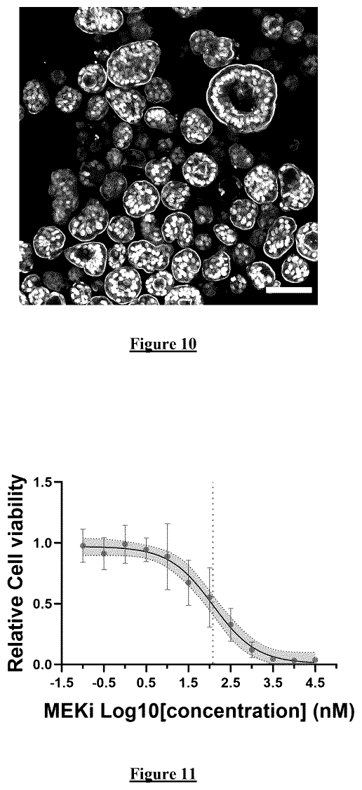

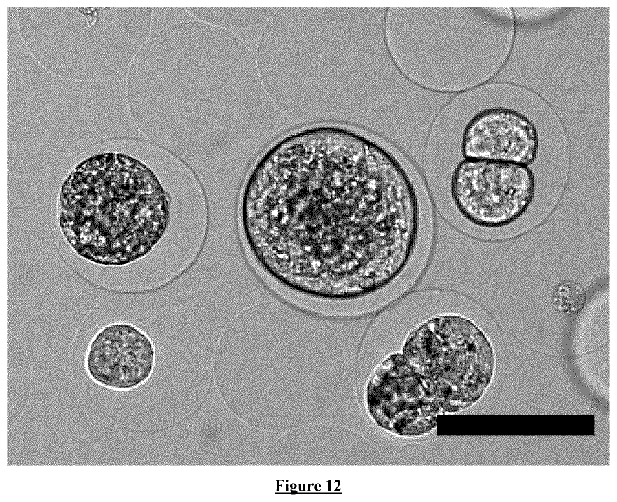

The results are illustrated by FIGS. 8, 9 and 10.

The results clearly demonstrate that Dextran-VS hydrogel effectively supports the growth and differentiation of organoids. As illustrated in FIG. 8, the presence of goblet cells, stem cells, and progenitor cells confirms the successful formation of intestinal-like structures. Examples 3.2 and 3.3 highlight the ability to generate both physiologically and non-physiologically structured organoids simply by altering the hydrogel composition-without modifying the overall culturing or encapsulation protocols. The apical-out organoids enable also the direct evaluation of the toxicity of the drug on the intestinal lumen.

Notably, apical-out organoids present a promising model for advanced drug testing, enabling the simulation of drug diffusion from the intestinal lumen, thereby mimicking peroral drug administration. In contrast, apical-in organoids preserve native tissue architecture, making them suitable for studies requiring physiological tissue morphology.

FIGS. 9 and 10 further underscore the consistency of organoid polarity across samples, demonstrating the robustness and reproducibility of our protocol. Additionally, our high-throughput method facilitates large-scale organoid production and analysis without the need for traditional well plates. This significantly reduces manual labor and procedural complexity, while enabling an exceptionally high-throughput organoid generation.

These findings open new avenues for applying similar protocols to PDOs (patient-derived organoids), PDXs (patient-derived xenografts), CDXs (cell line-derived xenografts), and even human biopsy samples, allowing for the faithful reproduction of their complexity and diversity within the hydrogel bead system as described in the present disclosure. Moreover, the inventor's approach reduces reliance on natural ECMs (extracellular matrices) such as

Matrigel™, minimizing animal-derived components and mitigating batch-to-batch variability. This approach not only offers a viable alternative to Matrigel™ but also allows efficient use of limited cell quantities, making it accessible and scalable for various research applications.

### Example 4: HIPSCs (Human Induced Pluripotent Stem Cells)

### 4.1 Materials and Methods

### 4.1.1 Used Products

1. hESC-Qualified Matrigel™ (Corning, 354277)
2. Cardiomyocyte Differentiation Kit (05010, StemCell Technologies)
3. Y-27632 (Tocris, 1254): ROCK INHIBITOR
4. Gentle Cell Dissociation Reagent (StemCell Technologies, 100-0485)
5. mTeSR™ + cell medium (StemCell Technologies, 100-0276)
6. ReLeSR™ (StemCell Technologies, 100-0483)
7. DMEM/F-12 (Gibco, 11320033)

### 4.1.2 Used Cells:

1. hiPSC: SCTi003-A (StemCell Technologies)
   4.2 Plate Coating with Matrigel™

Plate coating was performed according to Corning's protocol for human embryonic stem cell (hESC) culture. Briefly, one aliquot of hESC-qualified Matrigel™ (volume specified in Coming's quality certificate) was thawed on ice and diluted in 25 mL of cold DMEM/F-12. To coat a 6-well plate, 1 mL of the diluted Matrigel™ solution was added to each well. The plate was then incubated at room temperature for 1 hour. Prior to use, the remaining liquid was aspirated from each well.

4.3 hiPSC Culture

Human induced pluripotent stem cell (hiPSC) culture and cryopreservation were performed according to the guidelines provided by Stemcell Technologies. Briefly, frozen hiPSCs were thawed and centrifuged at 300 g for 5 minutes. The supernatant was aspirated, and the cell pellet was resuspended in mTeSR™+medium supplemented with 10 μM Y-27632 (prepared from a 10 mM stock solution in PBS).

Cells were then seeded onto hESC-qualified Matrigel™ coated plates (prepared as described in paragraph 4.2 above). After 24 hours, the medium was replaced to remove Y-27632. Subsequently, medium changes were performed every other day.

Healthy hiPSC colonies were identified by their dense, multilayered cores and well-defined borders. Passaging was carried out when cultures reached 80-90% confluence. Wells were first washed with PBS, followed by the addition of 1 mL ReLeSR™. After 1 minute, the ReLeSR™ was aspirated, and the plate was incubated at 37° C. for an additional 5 minutes. Cells were then detached by adding fresh medium, collected, and centrifuged at 300 g for 5 minutes. The resulting pellet was resuspended in mTeSR™ Plus and seeded onto fresh Matrigel™ coated plates.

4.4 hiPSC Preparation for Encapsulation

At least 4 hours prior to encapsulation, the culture medium was replaced with mTeSR™+supplemented with 10 μM Y-27632 to enhance cell viability during the encapsulation process. From this point onward, the ROCK inhibitor was maintained throughout the procedure.

For encapsulation, cells were first washed with PBS and then dissociated using Gentle Cell Dissociation Reagent for 8 to 12 minutes. Cell detachment was monitored under a microscope starting at 8 minutes to determine the optimal dissociation time.

Detached cells were collected in mTeSR™+containing 10 μM Y-27632 and centrifuged at 300 g for 5 minutes. The resulting pellet was resuspended at 10M cell/mL concentration for the encapsulation process.

### 4.5 Encapsulation Solution Preparation

The first solution was prepared with 124.6 μL of hiPSC solution at 10M/mL (prepared as described in paragraph 4.4 above) and 25.4 μL of Dextran-VS (DS=22.8%, solution, prepared at 20% w/v in PBS) prepared according to paragraph 1.1.12 above. The second one was prepared with 18 μL of SH-PEG-SH (1.5 kDa, 30% w/v in PBS) prepared according to paragraph 1.1.13 above and 6 μL of hESC-Qualified Matrigel™ in 126 μL of mTeSR™ +containing 10μ M Y-27632.

4.6 Encapsulation of hiPSC

hiPSC in dextran solution (prepared as described in paragraph 4.5 above) were encapsulated following the procedure outlined in paragraph 1.1.9 above, using a mixture of SH-PEG-SH prepared according to paragraph 1.1.13 above and Matrigel solutions.

4.7 Recuperation of Hydrogel Beads with hiPSC from Oil

The same process as that described in paragraph 1.1.10 above was applied using mTeSR™+containing 10 μM Y-27632 like a cell media.

4.8 Culturing hiPSC Encapsulated in Hydrogel Beads

The same procedure as that described in paragraph 1.1.11 above, but under shaking at 90rmp. With the mTeSR™+containing 10 μM Y-27632 as a culture media that was changed every day for the first 72 h post encapsulation, after that ROCK-inhibitor was removed from the cell media, and up to 7 days medium was changed every 2 days.

4.9 Differentiation of hiPSCs to Cardiomyocytes

hiPSCs encapsulated in hydrogel beads were maintained in culture for 7 days, as described in paragraph 4.6 above, to promote the formation of spheroids. The maintenance of pluripotency was confirmed on day 7 by the IF staining of the pluripotency markers SOX2 and NANOG (FIG. 13A).

Following this stage, guided differentiation was initiated using the Cardiomyocyte Differentiation Kit, following the manufacturer's protocol as detailed on the product's dedicated documentation page. The spheroids were cultured for 20 days following the protocol and then characterized by IF staining for alpha-smooth muscle actin (α-SMA) (FIG. 13B) and beating capacity (FIG. 13C).

4.10 IF of hiPSC Spheroids

Protocol is the same as described in 1.10 above with the use of primary antibodies:

| Antibody | Dilution | Reference |
|---|---|---|
| SOX2 | 1/100 | R&D systems, AF2018 |
| NANOG | 1/200 | Sigma, N3038 |
| α-SMA | 1/400 | Sigma, A5228 |

And the secondary:

| Antibody | Dilution | Reference |
|---|---|---|
| Donkey anti-Mouse | 1/500 | Thermo Fisher, A31571 |
| Donkey anti-Goat | 1/500 | Thermo Fisher, A21432 |

### 4.11 Results and Conclusions

The results clearly demonstrated that the inventors are capable of cultivating hiPSC spheroids using their hydrogel system described in the present disclosure. The hydrogel supports cell viability, adherence, and the formation of embryoid bodies, while maintaining pluripotency as confirmed by SOX2 and NANOG expression Mechanically and biologically, the hydrogel proved suitable for sustaining the growth of hiPSCs, which are typically challenging to culture, and even supported the development of beating cardiomyocytes. This work highlights the potential of the inventor's system described in the present disclosure for the efficient and high-throughput production of hiPSC-derived organoids, offering promising applications in drug testing and personalized medicine.

### Example 5: Reinjection of Spheroids

### 5.1 Spheroids Production

Spheroids from MKN45 cell line were produced according to example 1.2 above.

### 5.2 Spheroids Reinjection

Spheroids suspended in their cell culture medium were first centrifuged at 30 g in a 1.5 mL protein low-bind tube. After centrifugation, the supernatant beneath the hydrogel containing the spheroids was carefully removed.

A metal tube cap (P-CAP) with attached tubing was then used to connect the 1.5 mL protein low-bind tube to the pressure controller. This tubing was subsequently connected to the microfluidic chip.

All other inlets of the chip were supplied with either cell culture medium or oil, delivered via syringe pumps.

### 5.3 Results

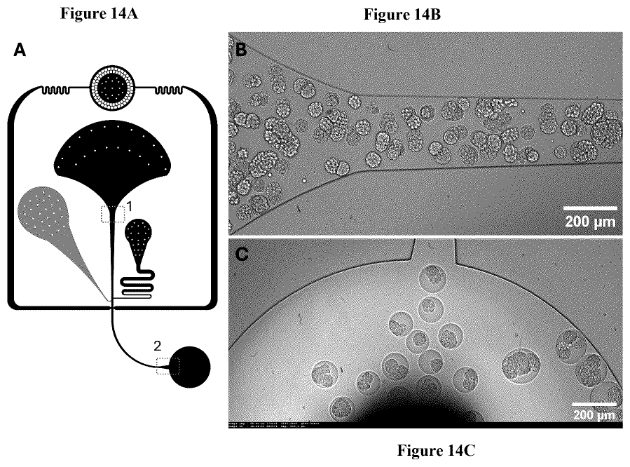

As shown in FIG. 14A, two key zones of the microfluidic chip are highlighted: (1) the compaction zone, where initial spheroid alignment occurs, and (2) the chip outlet, where encapsulated spheroids exit the device.

The hydrogel shell safeguards the spheroids from mechanical dissociation and facilitates smooth transit through the microfluidic channel, preventing clogging, as shown in FIG. 14B.

As shown in FIG. 14C, the hydrogel shell mitigates shear stress during droplet formation and enables compaction of spheroids allowing encapsulation techniques beyond Poisson distribution, ensuring more controlled and efficient spheroid encapsulation.

### 5.4 Conclusions

Using a hydrogel shell protects fragile 3D cell cultures from the mechanical stress induced by the encapsulation process. Moreover, it makes it possible to overcome Poisson-distributed loading during droplet encapsulation by compacting the biological entities protected by the hydrogel shell. This compaction is achieved through the hydrogel shell's sliding along the hydrophobic walls of the microfluidic device's channels, and it enables an increased yield of the encapsulation process.

### Example 6: Cell Centering in a Hydrogel Bead

### 6.1 Materials and Methods

### 6.1.1 Solution Preparation for Encapsulation.

Cells were detached with TrypLE™ Select (Gibco, ref. 12563011), and concentrated to about 1e7 cells/mL (that is to say 10⁷ cells/mL) in encapsulation buffer to allow approximately one cell per droplet. Encapsulation buffer consists of RPMI1640 GlutaMax (Gibco, 6187-010), supplemented with FBS (Gibco, A3161001) and 100 mM of HEPES (Gibco, 15630-080), adjusted to pH 7.6 with 1 M NaOH and 1M HCl. Cells were filtered through 37 μm strainer (Stemcell, 27215). Viability of the cells were estimated using Vi-Cell (Beckman Coulter), for all the experiment cells with viability higher than 90% were used. After that, depending on the desired recipe of the hydrogel, appropriate amount of dextran-VS DS=22.8% (20% (w/w) prepared according to paragraph 1.1.12 above in DPBS, was added to cells, and second equal volume solution of linker was prepared mixing appropriate amount of 30% (w/w) in water solution of SH-PEG-SH prepared according to paragraph 1.1.13 above (1.5 kDa, Merck, JKA4105) with encapsulation buffer.

TABLE 1
Preparation of 300 μL of hydrogel beads.

| | Dextran + Cells + | | SH-PEG-SH + Encapsulation | |
|---|---|---|---|---|
| | Encapsulation Buffer (first | | Buffer (second aqueous | |
| | aqueous inlet in chip) | | inlet in chip) | |
| | Dextran | | SH-PEG-SH | |
| Percentage | (20% (w/v) | Cell + | (30% (w/v) | |
| (w/v) of | in water), | Encapsulation | in water), | |
| the gel | μL | Buffer, μL | μL | Buffer, μL |
| 2.8% | 31.25 | 118.75 | 7.17 | 142.83 |
| 4.4% | 39.07 | 110.93 | 17.92 | 132.08 |

### 6.1.2 Cell Encapsulation

Cells with dextran solution, and PEG-linker solution were injected in droplet making chip, using syringe pump (Nemesys, Cetoni GmbH) and gas-tight glass syringes (Hamilton). Continuous phase was composed of HFE7500 (3M™) with the 2% of 008-Fluorosurfactant (RAN Biotechnologies). Both aqueous solutions generally were injected at 400 μL/h and continuous phase at 1200 μL/h allowing 1000 Hz production of 75 μm droplets. Produced droplets were collected through 5m long PTFE tubing with inner diameter of 0.3 mm (Adtech, ThermoFisher Scientific) rounded around paralepidid block of aluminum heated by Peltier's element to 37° C., allowing polymerization of droplets to the beads and centering of the cells in droplets. After the end of production, the rest of the beads in the PTFE tube were transferred to collection tube with the system perfusion by fresh HFE7500 w/o surfactant, and beads were left for 15 mins at 37° C. to allow complete polymerization.

### 6.1.3 Clusterized Bioassay

The cell line used for the clustering bioassay was a Jurkat/NFκB/eGFP (TR850A-1, Ozyme). To activate the NFκB pass way and to induce eGFP production (will generate green fluorescence) the CD3 receptor with OKT3 antibody an anti-CD3 (16-0037-85, eBioscience) were clusterized. The cell encapsulation protocol was the same that describes previously, and the hydrogel preparation was a w/v of gel of 4.4% (see Table 1, line 2). After encapsulation of the Jurkat cells in hydrogel beads, with or without in-line polymerisation, the cells were incubated in CTS optimizer (A1048501, ThermoFischer) for 18 hours. To clusterized the CD3 receptors, at the beginning of the experiment 20 nM of OKT3 antibody were added. Finaly to quantify the Jurkat activation, the green fluorescent intensity was read in a flow cytometer and define the percentage of cells activated versus none activated.

### 6.2 Results

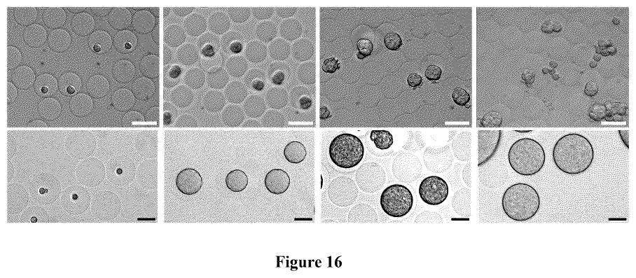

The inventor's initial experiments have shown that during encapsulation and subsequent polymerization in an Eppendorf tube, cells have sedimented and have shifted away from the center of the droplet. As a result, their growth has led to their escape from the droplet, significantly limiting the maximum size of the resulting spheroids. Moreover, this has made the spheroids more fragile during pipetting and centrifugation (FIG. 16, top row).

The solution was the implementation of a long PTFE capillary tube at the chip outlet, wrapped around a Peltier element heated to 37° C. The tube length was calculated to ensure that the droplet remained in the flow for approximately 10 minutes, which was sufficient for the polymerization of the dextran hydrogel at 37° C. After applying this solution, cells were more centered within the droplets, enabling the spheroids to grow larger until they eventually disrupt the gel structure (FIG. 16, bottom row).

Cells have proliferated within the gel matrix, stretching it as they grew. The inventors have observed that the spheroid size often exceeded the initial droplet size. For example, droplets with a 75 μm diameter could produce spheroids up to 200 μm. However, when the spheroid size greatly surpassed the droplet size, the cells often ruptured the gel. Proper centering of the cells within the droplets mitigated this issue, allowing for the formation of larger spheroids without compromising the gel's integrity.

Surprisingly, the inventors also observed an increase of the cell viability when the cells were centered in the hydrogel beads (FIG. 17.A).

The inventors have also investigated the possibility to clusterize membrane receptors to induce a cell pathway activation. They used the Jurkat cell models to do this. This cell model has become fluorescent when the CD3 proteins membranes were clusterized. When the inventors tried to clusterize the CD3 on the Jurkat Cell in the hydrogel beads according to the present disclosure, the fluorescent activation was very low (FIG. 17B, see the bar or column in the middle of the graph) compared to the cells in normal condition (FIG. 17B, see bar or column on the left of the graph). Surprisingly, when the inventors have carried out the same experiment with in-line polymerization process, they increase drastically the efficacity of clusterization (FIG. 17B, see the two bars or columns in the middle and on the right of the graph).

### 6.3 Discussion

The inventors' method for centering cells within droplets offers a simpler solution compared to the re-encapsulation of hydrogel beads into droplets. Unlike re-encapsulation, their approach avoids the retreatment of empty droplets, which lengthens the re-encapsulation process, and eliminates the need for reapplying Poisson distribution to the beads. Furthermore, even when re-encapsulation methods with hydrogel bead compaction are employed, the secondary passage through a microfluidic chip remains a complex challenge. Additionally, the inventors' method does not require the design of a specialized microfluidic chip and can be applied to any existing setup. Unlike polymerization methods using planetary shakers at high-speed, their approach is a flow-based method, enabling the production of polymerized particles with centered cells immediately after droplet generation, and ensuring gentle polymerization conditions.

Additionally, the inventors' method of in-line polymerization enables a better viability that could probably be explained by a higher protection of the hydrogel layer when the cell is centering, compare when cells are in the border of the hydrogel bead. This uniform hydrogel layer has probably a good protection again shear stress and enables also to avoid the cell to escape easily the hydrogel beads.

Additionally, their method of in-line polymerization enables a better clusterization. The inventors hypothesized that the in-line polymerization process increases the porosity of the hydrogel compared to the same hydrogel without in-line polymerization, enabling the protein membranes to move that allowed the clusterization of them.

### 6.4 Conclusion

Cell centering within beads enables the formation of larger spheroids and organoids using smaller droplets, thanks to the uniform distribution of gel around the cells. Reducing droplet size during production increases the manufacturing speed, thereby accelerating the overall process. Additionally, the symmetry of gel placement around the spheroid or organoid is expected to simplify subsequent protocols involving these structures. This includes potential re-encapsulation, robotic non-contact and contact pipetting and distribution, or the use of such structures in flow-based analysis methods, such as FACS-like (fluorescence activated cell sorting-like) machines for large objects, including the COPAS Flow Cytometer (Union Biometrica).

The in-line polymerization had demonstrated numerous advantages. By increasing the cell viability, it enables an increase of the yield of spheroids production. By allowing the clusterization of membrane protein, it opens the possibility to perform functional cell assay with high throughput and at the single cell level. This functional assay is for example intensively used to discover agonist antibodies or any agonist molecules (proteins, small molecules, etc.).

### Example 7: Yeast Encapsulation and Growth in Hydrogel Beads According to the Present Disclosure

### 7.1 Materials and Methods

### 7.1.1 Used Products

1. SH-PEG-SH-2000 Da (BD01417719, BLDpharm)
2. YNBww (233520, Difco)
3. Glucose (G7021, Sigma-Aldrich)
4. Ultra-low IgG FBS (Gibco, A33819-01)
5. GlutaMax (Gibco, 35050-038)
6. DMEM-F12 (Gibco, 21041-025)
7. RPMI1640 (Gibco, 32404-014)
8. Yeast Extract (Difco, 210929)
9. 5 μm filter (43-50005 Jan. 1, PluriSelect)

### 7.1.2 Used Cells:

1. Yeast: Y. lipolytica (Yarrowia lipolytica)

### 7.2 Preparation of Encapsulation Media

The encapsulation medium used is the same as that described in Lebrun et al, Efficient full-length IgG secretion and sorting from single yeast clones in droplet picoreactors, Royal Society of Chemistry, Lab on a Chip, 2023, 23, 3487-3500. Briefly, to prepare the encapsulation medium it is needed to take 1.3 mL of YNB (1.7 g/L of YNBww, 5.0 g/L NH4C1, 50 mM phosphate buffer (pH 6.8), 5 g/L glucose), 1 mL of Lysine solution (8 g/L), 5 mL of Ultra-low IgG FBS, 0.13 mL of GlutaMax, 8.8 mL of DMEM-F12 and 8.8 mL of RPMI1640.

### 7.3 Preparation of Growth Media

The growth medium used is the same as that described in Lebrun et al, Efficient full-length IgG secretion and sorting from single yeast clones in droplet picoreactors, Royal Society of Chemistry, Lab on a Chip, 2023, 23, 3487-3500. Briefly, medium was prepared using, 10 g/L yeast extract, 10 g/L peptone, 10 g/L glucose.

### 7.4 Yeast Preparation for Encapsulation

Yeasts were prepared as described in Lebrun et al, Efficient full-length IgG secretion and sorting from single yeast clones in droplet picoreactors, Royal Society of Chemistry, Lab on a Chip, 2023, 23, 3487-3500. Briefly, yeasts were centrifuged at 2000 g and filtered through 5 μm filter, and concentration is adjusted to optical density (OD) 1 using Cell density meter model 40 (Fisher Scientific).

### 7.5 Encapsulation Solution Preparation

A 2.8% (w/v) gel was used. To obtain 300 μL of this gel, 28.81 μL of Dextran-VS (DS=21.8%) at 20% (w/v) prepared according to paragraph 1.1.12. above, 8.79 μL of SH-PEG-SH-2000 Da at 30% (w/v) prepared according to paragraph 1.1.13. above and 262.4 μL of the encapsulation buffer with cells were added. The size of the thus produced droplets was around 50 μm.

### 7.6 Encapsulation of Yeast

Yeast in dextran solution (as described above) were encapsulated following the procedure outlined in paragraph 1.1.9 above, using a mixture of SH-PEG-SH-2000 Da (as described above).

7.7 Recuperation of Hydrogel Beads with Yeast from Oil

Same as described in 1.1.10 above using growth medium.

### 7.8 Culturing Yeast Encapsulated in Hydrogel Beads

Beads with yeasts were incubated in growth medium at 28° C.

### 7.9 Results and Conclusions

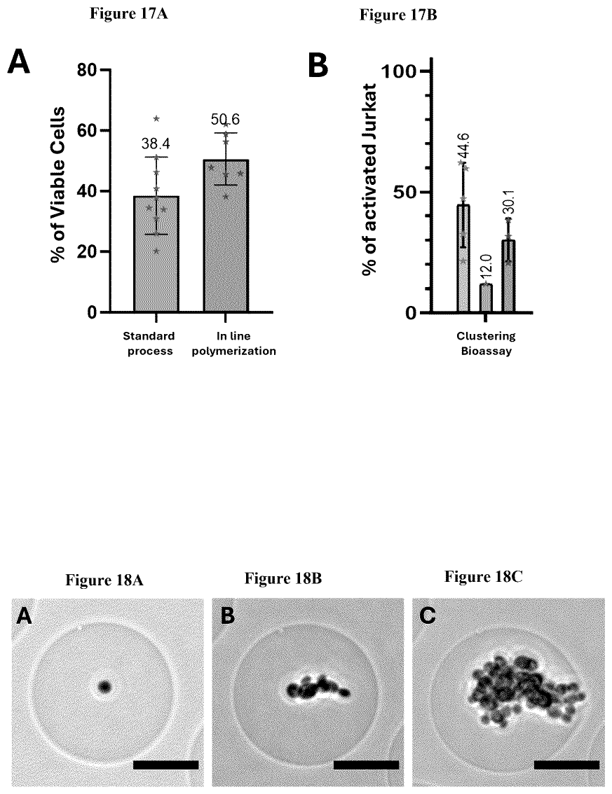

As shown in FIGS. 18A, 18B and 18C, the inventors have demonstrated that the growth of yeasts can also be carried out in hydrogel beads according to the present disclosure. Also, after the encapsulation, a part of it was treated by dextranase to dissolve the gel around the yeast cells and to prepare them for cultivation at OD0.01 for further culture, and after 18 h the OD of yeast cells culture in growth media was 0.73 showing the lack of effect of dextranase treatment for viability and proliferation of cells.

1. Use of modified dextran hydrogel microparticles comprising (i) at least one vinyl sulfone functionalized dextran, and (ii) at least one crosslinkable polymer having at least two thiol functions, wherein
the dextran has a molecular weight comprised between 5 and 500 kDa, and
the substitution degree of the dextran by the vinyl sulfone is comprised between 5 and 60%, for encapsulating at least one synthetic or natural cell.

2. The use according to claim 1, wherein the molecular weight of the dextran is comprised between 40 and 150 kDa, in particular between 50 and 100 kDa, for example is 70 kDa.

3. The use according to claim 1 or 2, wherein the substitution degree of the dextran by the vinyl sulfone is comprised between 10 and 40%, in particular between 18 and 38%.

4. The use according to any one of the preceding claims, wherein the crosslinkable polymer comprises
at least a polymer chain selected from a group consisting of PEG, dextran, gelatin, agarose, alginate, hyaluronic acid, collagen, proteins, polyacrylamide, block-polymers, at least an (C4-C12)alkylene chain, optionally substituted by one or two hydroxy group(s), or
their mixtures,
in particular PEG.

5. The use according to any one of the preceding claims, wherein the crosslinkable polymer is selected from a group consisting in a 2 to 12 arms polyethylene glycol thiol, dithiothreitol, dextran-SH, gelatin-SH, collagen-SH, proteins having at least two thiol functions and block-polymers having at least two thiol functions, in particular a PEG dithiol or a four-arm PEG thiol.

6. The use according to any one of the preceding claims, wherein the at least one synthetic or natural cell is selected from eukaryotic cells such as a mammalian cells, for instance human cells, prokaryotic cells such as bacterial cells or archaea cells, cancerous cells, tumoral cells, somatic cells, spleen cells, stem cells, progenitor cells, precursor cells, fully differentiated cells, undifferentiated cells, germ cells, cells of unicellular organism or of multi-cellular organism, fungal cells, splenocytes and hybridoma cell.

7. The use according to any one of the preceding claims, wherein the at least one synthetic or natural cell is selected from the group consisting of connective tissue cells, epithelial cells, muscle cells, blood cells, neuronal cells, endothelial cells, fibroblasts, keratinocytes, smooth muscle cells, stromal cells, mesenchymal cells, cord blood cells, embryonic stem cells, induced pluripotent stem cells (IPSc), patient-derived stem cells (PDSC), PDX cells (patient-derived xenograft cells), CDX cells (cell line-derived xenograft), biopsy samples cells, PDO cells (patient-derived organoid cells), placental cells, bone cells, bone marrow derived cells, immune system cells, hematopoietic cells, dendritic cells, hair follicle cells, chondrocytes, cardiomyocytes, hybridoma cells, cells from specific cell lines such as CHO cell, Jurkat cell, PreB cell, NK92 cell, THPIB cell, cell-free systems such as cell extract-based cell free systems and purified enzyme-based cell free systems and combinations thereof.

8. The use according to any one of the preceding claims, wherein the weight ratio of vinyl sulfone functionalized dextran and crosslinkable polymer having at least two thiol functions is comprised between 1% and 30%, in particular between 2% and 20%, particularly between 2.5% and 10%.

9. The use according to any one of the preceding claims, wherein the size of the modified dextran hydrogel microparticles is between 1 and 1000 μm, in particular between 1 μm and 999 μm, in particular between 10 and 500 μm, in particular between 30 and 180 μm.

10. The use according to any one of the preceding claims, wherein the modified dextran hydrogel microparticles has a coefficient of variation in size lower than 50%, in particular lower than 20%, and more particularly lower than 5%.

11. The use according to any one of the preceding claims, wherein it is aimed at cultivating said at least one synthetic or natural cell, for example as a 3D cell culture, in particular by incubating the microparticles in an environment suitable to allow for cell survival, cell growth, cell differentiation and/or cell proliferation such as clonal expansion, more particularly for producing a cell cluster, a spheroid, an organoid, a gastruloid, a tumoroid or a tissue.

12. The use according to any one of claims 1 to 10, wherein it is aimed at screening or discriminating compounds, proteins, polypeptides, oligopeptides, nucleic acids, cell secretome or antibodies, in particular by performing RNA-sequencing, by using cell staining such as chemical or immunostaining or by using imaging such as electronic imaging or optic imaging.

13. Modified dextran hydrogel microparticles comprising (i) at least one vinyl sulfone functionalized dextran, (ii) at least one crosslinkable polymer having at least two thiol functions and (iii) at least one synthetic or natural cell, wherein
the dextran has a molecular weight comprised between 5 and 500 kDa, and
the substitution degree of the dextran by the vinyl sulfone is comprised between 5 and 60%, provided that the at least one crosslinkable polymer having at least two thiol functions is different from a crosslinkable polymer comprising dextran having a molecular weight comprised between 30 and 50 kDa as the polymer chain.

14. Modified dextran hydrogel microparticles comprising (i) at least one vinyl sulfone functionalized dextran, (ii) at least one crosslinkable polymer having at least two thiol functions and (iii) at least one synthetic or natural cell,
wherein
the dextran has a molecular weight comprised between 5 and 500 kDa,
the substitution degree of the dextran by the vinyl sulfone is comprised between 5 and 60%, and
the crosslinkable polymer comprises
at least a polymer chain selected from a group consisting of PEG, gelatin, agarose, alginate, hyaluronic acid, collagen, proteins, polyacrylamide, block-polymers,
at least an (C4-C12)alkylene chain, optionally substituted by one or two hydroxy group(s), or
their mixtures,
in particular PEG.

15. A process for preparing modified dextran hydrogel microparticles according to claim 13 or 14, wherein it comprises at least a step of using a microfluidic device, in particular comprising at least two inlet channels that converge into a droplet generation region.

16. The process for preparing modified dextran hydrogel microparticles according to the preceding claim, wherein it comprises at least the following steps of:
(a) Providing a first solution which comprises the at least one synthetic or natural cell, in an encapsulation buffer and at least one vinyl sulfone functionalized dextran:
(b) Providing a second solution which comprises an encapsulation buffer, identical or different from the buffer used in step (a), and the at least one crosslinkable polymer having at least two thiol functions:
(c) Forming water-in-oil droplets by encapsulating in co-flow through the two inlet channels the first and second solutions of steps (a) and (b) and an oily phase:
(d) Polymerizing the water-in-oil droplets at a temperature comprised between 0.01 and 99° C., in particular between 1 and 50° C., for instance at 4° C., room temperature or 37° C. to obtain the modified dextran hydrogel microparticles in oil:
(d′) Optionally implementing during said step (d) a capillary tube at a chip outlet, said tube being long enough to allow a polymerization, in particular a total or a partial polymerization of the water-in-oil droplets, and said tube being in contact with a heating element (solid, liquid or gaseous), in particular said tube being wrapped around a heating element or in particular said tube being immerged in a water bath, at a temperature comprised between 0.01 and 99° C., in particular between 1 and 50° C., for instance at 4° C., room temperature or 37° C.; and
(e) Optionally extracting the modified dextran hydrogel microparticles from the oily phase by washing, extracting, demulsification, electric fields destabilization, chemical destabilization and/or oil evaporation, for obtaining modified dextran hydrogel microparticles in an aqueous solution.

17. The process according to any one of claim 15 or 16, wherein the w/v percentage of the at least one vinyl sulfone functionalized dextran and the at least one crosslinkable polymer is comprised between 1% and 30%, in particular between 2% and 15%, and more particularly between 2.5% and 12%, for example between 2.8% and 10% with respect to the volume of the first and second solutions of step (a) and (b) according to claim 16.

18. Modified dextran hydrogel microparticles comprising (i) at least one vinyl sulfone functionalized dextran, (ii) at least one crosslinkable polymer having at least two thiol functions and (iii) at least one synthetic or natural cell obtainable by a process according to any one of claims 15 to 17.

19. Modified dextran hydrogel microparticles according to any one of claim 13, 14 or 18, wherein a bulk modified dextran hydrogel formed by reacting the at least one vinyl sulfone functionalized dextran, in particular according to claim 2 or 3 and the at least one crosslinkable polymer having at least two thiol functions, in particular according to claim 4 or 5 in the same weight ratio as the one used for forming said microparticles, presents a Young's modulus (elastic modulus) comprised between 10 and 50 kPa, in particular from 20 to 30 kPa; more particularly from 20 to 25 kPa.

20. Modified dextran hydrogel microparticles according to any one of claim 13, 14, 18 or 19, wherein it presents a porosity up to 200 kDa less than 700 kDa, as quantified by analyzing the diffusion of fluorescently labeled molecules of various molecular weights through the hydrogel matrix.

21. An in vitro method for cultivating at least one cell comprised in the modified dextran hydrogel microparticle as claimed in any one of claim 13, 14, 18, 19 or 20, comprising at least a step of incubating the microparticle in an environment suitable to allow for cell survival, cell growth, cell differentiation and/or cell proliferation such as clonal expansion, more particularly for producing a cell cluster, a spheroid, an organoid, a gastruloid, a tumoroid or a tissue.

22. An in vitro method for screening compounds, proteins, polypeptides, oligopeptides, nucleic acids, cell secretome or antibodies comprising using a modified dextran hydrogel microparticle comprising cultivated cells according to the in vitro method according to claim 21.

23. A kit for making the microparticle encapsulating at least one synthetic or natural cell according to any one of claim 13, 14, 18, 19 or 20, the kit comprising: at least one vinyl sulfone functionalized dextran, a culture medium; at least one crosslinkable polymer having at least two thiol functions: an oily phase: a microfluidic chip; and optionally instructions for use.

24. An in vitro method for producing compounds, proteins, polypeptides, oligopeptides, nucleic acids, cell secretome or antibodies from at least one synthetic or natural cell encapsulated in the modified dextran hydrogel microparticle(s) as defined in any one of claims 13, 14, 18, 19, and 20, comprising at least:
a step of cultivating and/or of operational maintaining of the at least one synthetic or natural cell: or
a step of production of said compounds, proteins, polypeptides, oligopeptides, nucleic acids, cell secretome or antibodies from said at least one synthetic or natural cell: or
a step of cultivating and/or of operational maintaining of the at least one synthetic or natural cell, and a step of production of said compounds, proteins, polypeptides, oligopeptides, nucleic acids, cell secretome or antibodies from said at least one synthetic or natural cell.

25. A microfluidic or millifluidic circuit or channel comprising the modified dextran hydrogel microparticles as defined in any one of claims 13, 14, 18, 19, and 20.

26. The microfluidic or millifluidic circuit or channel according to claim 25, wherein said modified dextran hydrogel microparticles are flowing and/or are stored inside said microfluidic or millifluidic circuit or channel.

27. A process for encapsulating in a compartment, in particular in an aqueous or in a hydrogel droplet, a modified dextran hydrogel microparticles as defined in any one of claims 13, 14, 18, 19, and 20 comprising at least one step of injection of said modified dextran hydrogel microparticles in a microfluidic or millifluidic circuit or channel.

28. The modified dextran hydrogel microparticle as defined in any one of claim 13, 14, 18, 19 or 20 encapsulated in a compartment, in particular in an aqueous or a hydrogel droplet.

29. A method for the quality control of a batch of modified dextran hydrogel microparticles as defined in any one of claim 13, 14, 18, 19 or 20, comprising at least:
a step of recovering of a sample of microparticles from said batch:
a step of measuring at least one parameter for each microparticle of said sample:
a step of comparison of the value of the at least one measured parameter for each microparticle of said sample with a predetermined value:
a step of determination based on said comparison whether or not each microparticle of said sample has the required quality; and
a step of extrapolation of the results of the previous determination step to said batch.

30. The method according to claim 29, wherein the at least one parameter is selected from the viability of the at least one natural or synthetic cell, the morphology of the at least one natural or synthetic cell, the potency of the at least one natural or synthetic cell, the presence, the quantity and the sequence of RNA molecules in the at least one natural or synthetic cell, the presence and/or the concentration of at least one compound, protein, polypeptide oligopeptides, nucleic acid, cell secretome or antibody secreted by the at least one natural or synthetic cell.

31. A pharmaceutical composition for use in a method of treating a patient having a disorder or condition, and/or for transplantation therapy, and/or for use to restore and/or improve the function of a tissue or of an organ of a patient, and/or for use for tissue regeneration and/or tissue repair,
said pharmaceutical composition comprising the cells cultivated according to the in vitro method as defined in claim 21.

32. An in vitro method for testing a drug compound comprising using at least one cell, cell cluster, spheroid, organoid, gastruloid, tumoroid or tissue as defined in claim 11, in particular said at least one cell, cell cluster, spheroid, organoid, gastruloid, tumoroid or tissue comprising at least one synthetic or natural cell as defined in claim 7, produced in a modified dextran hydrogel microparticles as defined in any one of claim 13, 14, 18, 19 or 20.

33. An in vitro method for testing a drug compound comprising at least:
a step of culturing at least one cell, cell cluster, spheroid, organoid, gastruloid, tumoroid or tissue as defined in claim 11, in particular said at least one cell, cell cluster, spheroid, organoid, gastruloid, tumoroid or tissue comprising at least one synthetic or natural cell as defined in claim 7, produced according to the in vitro method as defined in claim 21,
a step of exposing said at least one cell, cell cluster, spheroid, organoid, gastruloid, tumoroid or tissue as defined in claim 11, in particular said at least one cell, cell cluster, spheroid, organoid, gastruloid, tumoroid or tissue comprising at least one synthetic or natural cell as defined in claim 7, to the drug compound,
a step of measuring a biological response of interest, and
a step of comparison of the biological response with a predetermined value or with a control condition wherein said at least one cell, cell cluster, spheroid, organoid, gastruloid, tumoroid or tissue as defined in claim 11, in particular said at least one cell, cell cluster, spheroid, organoid, gastruloid, tumoroid or tissue comprising at least one synthetic or natural cell as defined in claim 7, has not been exposed to said drug compound, thereby assessing the biological response of the tested drug compound.
